SEQUENCE LISTING (30 sequences)

SEQ ID NO: 1 - protein, 122 aa, Homo sapiens
VNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFM

SEQ ID NO: 2 - protein, 122 aa, Homo sapiens
AQELKEQGNRLFVGRKYPEAAACYGRVITRNPLVAVYYTNRALCYLKMQQHEQALADCRRALELDGQSVKAHFFLGQCQLEMESYDEAIANLQRAYSLAKEQRLNFGDDIPSALRIAKKKRW

SEQ ID NO: 3 - protein, 128 aa, Homo sapiens
ALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERL

SEQ ID NO: 4 - protein, 122 aa, Homo sapiens
ALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDS

SEQ ID NO: 5 - protein, 122 aa, Homo sapiens
AEELKTQANDYFKAKDYENAIKFYSQAIELNPSNAIYYGNRSLAYLRTECYGYALGDATRAIELDKKYIKGYYRRAASNMALGKFRAALRDYETVVKVKPHDKDAKMKYQECNKIVKQKAFE

SEQ ID NO: 6 - protein, 137 aa, Homo sapiens
AAIVKEKGTVYFKGGKYMQAVIQYGKIVSWLEMEYGLSEKESKASESFLLAAFLNLAMCYLKLREYTKAVECCDKALGLDSANEKGLYRRGEAQLLMNEFESAKGDFEKVLEVNPQNKAARLQISMCQKKAKEHNER

SEQ ID NO: 7 - protein, 137 aa, Homo sapiens
STIVKERGTVYFKEGKYKQALLQYKKIVSWLEYESSFSNEEAQKAQALRLASHLNLAMCHLKLQAFSAAIESCNKALELDSNNEKGLFRRGEAHLAVNDFELARADFQKVLQLYPNNKAAKTQLAVCQQRIRRQLAR

SEQ ID NO: 8 - protein, 138 aa, Homo sapiens
TEDLKNIGNTFFKSQNWEMAIKKYAEVLRYVDSSKAVIETADRAKLQPIALSCVLNIGACKLKMSNWQGAIDSCLEALELDPSNTKALYRRAQGWQGLKEYDQALADLKKAQGIAPEDKAIQAELLKVKQKIKAQKDK

SEQ ID NO: 9 - protein, 117 aa, Homo sapiens
ARVLKEEGNELVKKGNHKKAIEKYSESLLCSNLESATYSNRALCYLVLKQYTEAVKDCTEALKLDGKNVKAFYRRAQAHKALKDYKSSFADISNLLQIEPRNGPAQKLRQEVKQNLH

SEQ ID NO: 10 - protein, 121 aa, Homo sapiens
AAKLKELGNKAYGSKDFNKAIDLYSKAIICKPDPVYYSNRAACHNALAQWEQVVADTTAALKLDPHYVKALNRRANAYDQLSRYRHALLDFTASCIIDGFRNEQSAQAVERLLKKFAENKA

SEQ ID NO: 11 - protein, 126 aa, Homo sapiens
AENFKKQGNELYKAKRFKDARELYSKGLAVECEDKSINESLYANRAACELELKNYRRCIEDCSKALTINPKNVKCYYRTSKAFFQLNKLEEAKSAATFANQRIDPENKSILNMLSVIDRKEQELKA

SEQ ID NO: 12 - protein, 127 aa, Homo sapiens
STRLKEEGNEQFKKGDYIEAESSYSRALEMCPSCFQKERSILFSNRAAARMKQDKKEMAINDCSKAIQLNPSYIRAILRRAELYEKTDKLDEALEDYKSILEKDPSIHQAREACMRLPKQIEERNER

SEQ ID NO: 13 - protein, 126 aa, Homo sapiens
LKAKKEDGNKAFKEGNYKLAYELYTEALGIDPNNIKTNAKLYCNRGTVNSKLRKLDDAIEDCTNAVKLDDTYIKAYLRRAQCYMDTEQYEEAVRDYEKVYQTEKTKEHKQLLKNAQLELKKSKRKD

SEQ ID NO: 14 - protein, 122 aa, Homo sapiens
GELMKMKGNEEFSKERFDIAIIYYTRAIEYRPENYLLYGNRALCFLRTGQFRNALGDGKRATILKNTWPKGHYRYCDALSMLGEYDWALQANIKAQKLCKNDPEGIKDLIQQHVKLQKQIED

SEQ ID NO: 15 - protein, 126 aa, Homo sapiens
AKTYKDEGNDYFKEKDYKKAVISYTEGLKKKCADPDLNAVLYTNRAAAQYYLGNFRSALNDVTAARKLKPCHLKAIIRGALCHLELIHFAEAVNWCDEGLQIDAKEKKLLEMRAKADKLKRIEQRD

SEQ ID NO: 16 - protein, 122 aa, Homo sapiens
FKALKEEGNQCVNDKNYKDALSKYSECLKINNKECAIYTNRALCYLKLCQFEEAKQDCDQALQLADGNVKAFYRRALAHKGLKNYQKSLIDLNKVILLDPSIIEAKMELEEVTRLLNLKDKT

SEQ ID NO: 17 - protein, 122 aa, Homo sapiens
AERLKTEGNEQMKVENFEAAVHFYGKAIELNPANAVYFCNRAAAYSKLGNYAGAVQDCERAICIDPAYSKAYGRMGLALSSLNKHVEAVAYYKKALELDPDNETYKSNLKIAELKLREAPSP

SEQ ID NO: 18 - protein, 127 aa, Homo sapiens
AQAAKNKGNKYFKAGKYEQAIQCYTEAISLCPTEKNVDLSTFYQNRAAAFEQLQKWKEVAQDCTKAVELNPKYVKALFRRAKAHEKLDNKKECLEDVTAVCILEGFQNQQSMLLADKVLKLLGKEKA

SEQ ID NO: 19 - protein, 34 aa, Homo sapiens
VNELKEKGNKALSVGNIDDALQCYSEAIKLDPHN

SEQ ID NO: 20 - protein, 34 aa, Homo sapiens
ALKEKELGNDAYKKKDFDTALKHYDKAKELDPTN

SEQ ID NO: 21 - protein, 34 aa, Homo sapiens
ALEEKNKGNECFQKGDYPQAMKHYTEAIKRNPKD

SEQ ID NO: 22 - protein, 34 aa, Homo sapiens
HVLYSNRSAAYAKKGDYQKAYEDGCKTVDLKPDW

SEQ ID NO: 23 - protein, 41 aa, Homo sapiens
MTYITNQAAVYFEKGDYNKCRELCEKAIEVGRENREDYRQI

SEQ ID NO: 24 - protein, 34 aa, Homo sapiens
AKLYSNRAACYTKLLEFQLALKDCEECIQLEPTF

SEQ ID NO: 25 - protein, 32 aa, Homo sapiens
GKGYSRKAAALEFLNRFEEAKRTYEEGLKHEA

SEQ ID NO: 26 - protein, 32 aa, Homo sapiens
AKAYARIGNSYFKEEKYKDAIHFYNKSLAEHR

SEQ ID NO: 27 - protein, 32 aa, Homo sapiens
IKGYTRKAAALEAMKDYTKAMDVYQKALDLDS

SEQ ID NO: 28 - protein, 122 aa, Homo sapiens; identical to SEQ ID NO: 1

SEQ ID NO: 29 - protein, 128 aa, Homo sapiens; identical to SEQ ID NO: 3

SEQ ID NO: 30 - protein, 122 aa, Homo sapiens; identical to SEQ ID NO: 4

(19) United States
(12) Patent Application Publication
(10) Pub. No.: US 2003/0138848 A1
(43) Pub. Date: Jul. 24, 2003

(54) 3D structure of polypeptides containing a TPR-structure motif with chaperone-binding function, crystals thereof and compounds for inhibition of said polypeptides

(76) Inventors: Ismail Moarefi, Munchen (DE); Clemens Scheufler, Munchen (DE); Ulrich Hartl, Kottgeisering (DE); Achim Brinker, Planegg (DE)

Correspondence Address: KLAUBER & JACKSON, 411 HACKENSACK AVENUE, HACKENSACK, NJ, 07601

(21) Appl. No.: 10/262,525

(22) Filed: Sep. 30, 2002

Related U.S. Application Data
(63) Continuation of application No. PCT/EP01/03617, filed on Mar. 29, 2001.

(30) Foreign Application Priority Data
Mar. 29, 2000 (DE) 100 15 748.3
Apr. 13, 2000 (DE) 100 18 335.2

(51) Int. Cl. G01N 33/53; G06F 19/00; G01N 33/48; G01N 33/50; C12N 9/64

(52) U.S. Cl. 435/7.1; 435/226; 702/19

(57) ABSTRACT

The invention relates to 3-D structures of a polypeptide, whereby, in the 3-D structure, the polypeptide contains at least one amino acid sequence of a TPR-structure motif from a Hop protein, or a derivative thereof, The invention further relates to crystals, comprising at least one polypeptide in their asymmetric unit and, optionally, at least one further compound, whereby the polypeptide adopts the above 3D-structure in the crystal. Furthermore, methods for the production of such crystals and compounds, having the property of binding, as ligands, to structural regions of a Hop protein are disclosed. Additionally, a method for the identification of inhibitors is disclosed, whereby ligands with inhibitor functions for the interaction of a Hop protein with at least one chaperone protein are obtained. Such inhibitors are useful for the treatment of tumour disease states, immunosupression, GVDH, or the treatment of viral infections.

The present invention relates to 3D structures and space forms, respectively, of a polypeptide with a TPR-structure motif sequence, methods for structure determination of such polypeptides, crystals having such 3D structures, methods for production of such crystals, compounds having the ability to bind specific structure areas in a highly affined manner by means of such 3D structures, applications of such compounds to produce a medication, and applications of such compounds for medication used for specific medical indication, methods for the determination of such compounds, and applications of compounds obtainable by means of the above methods.

From prior art it is known that in a number of cellular signal proteins, a coordination of the effects of Hsp70 and Hsp90 chaperone proteins is required for the folding and conformational regulation of these signal proteins. Among the signal proteins, especially nuclear receptors of steroid hormones and several serine/threonine and tyrosin kinases having proto-oncogenic properties, such as Raf or Src, should be mentioned (Buchner, 1999, Trends Biochem. Sci. 24, 136-141; Caplan, 1999, Trends Cell. Biol. 9, 262-268; Pratt, 1997, Endocr. Rev. 18, 306-360).

Many polypeptide chains interact co-translationally with chaperone proteins of the Hsp70n family, developing their effect by prohibiting malformed folding and aggregation of nascent protein chains. Polypeptide chains, which have just synthesised at the ribosomes, are either released to unfold into their native conformations, without any help from other chaperone proteins, or they are passed on to a specifically designed chaperone system (Hartl, 1996, Nature 381, 571-579; Johnson and Craig, 1997, Cell 90, 201-204). Among these systems, which take effect further downstream in the eukaryotic cytosol, the chaperonin system TriC/CCT and the Hsp90 multi chaperone device were characterised. Hsp90 obtains its substrates from Hsp70 in a reaction which depends in a critical manner on the function of the Hop protein (a protein which organises Hsp70 and Hsp90, also known as p60 or stilp). In various papers (Chang et al., 1997, Mol. Cell. Biol. 17, 318-325; Chen and Smith, 1998, J. Biol. Chem. 273, 35194-35200; Frydman and Hohfeld, 1997, Trends Biochem. Sci. 22, 1718-1720; Johnson et al., 1998, J. Biol. Chem. 273, 3679-3686) it was shown that the Hop protein, being an adaptor protein, provides specific binding locations for the two above main chaperone proteins.

Honore et al. (1992 J. Biol. Chem. 267, 8485-8491) and Smith et al. (1993 Mol. Cell. Biol. 13, 869-876) were able to show that the Hop protein is almost exclusively made out of TPR (tetratrico peptide repeats) domains, but not being capable of independently developing any activity as a chaperone protein (Bose et al., 1996, Science 274, 1715-177; Freeman et al., 1996, Science 274, 1718-1720). From the paper by Lamb et al. (1995, Trends Biochem. Sci. 20, 257-259) it is known that TPR domains consist of three or more TPR structure motifs (having a length of approx. 34 amino acids), so that the TPR domains are distinguished by degenerated repeats with a length of 34 amino acids.

Based upon primary sequence data of the Hop protein, relevant literature predicted that the Hop protein contains nine TPR structure motifs, which in turn form two TPR domains. As a result of the analyses of deletion mutants it had turned out that the N-terminal TPR domain of Hop (TPR1 (three TPR structure motifs)) is responsible for the interaction with the C-terminal Hsp70, whereas a C-terminal TPR2 domain (six TPR structure motifs) basically ensures the interaction of the Hop protein with Hsp90 (Chen et al., 1996, Mol. Endocrinol. 10, 682-693; Demand et al., 1998, Mol. Cell. Biol.18, 2023-2028; Lassle et al., 1997, J. Biol. Chem. 272, 1876-1884). Prodromou et al. (1999, EMBO J. 18, 754-762) were able to show that the Hop protein inhibits the Hsp90 ATPase, blocking the access of ATP or the inhibitor geldanamycin to its binding pocket in the N-terminal domain of HSP90 (Stebbins et al., 1997, Cell 89, 239-250; Prodromou et al., 1997, Cell 90, 65-75, and 1999, EMBO J. 18, 754-762). Carello et al. (1999, J. Biol. Chem. 274, 2682-2689) and Young et al. (1998, J. Biol. Chem. 273, 18007-18010) assigned, just as in the case of the Hsp70 chaperone, the binding location of the Hop protein to the C-terminal domain of the Hsp90 chaperone. Thus, Chen et al. (1998, Cell Stress Chaperones 3, 118-129) were able to show that regarding Hsp90, the integrity of the conserved C-terminal EEVD motif could represent a crucial component for the interaction. From prior art it is also known that the C-terminal domain of Hsp90 binds a number of other TPR containing co-chaperones, where especially the large immunophiles Cyp-40, FKBP51 and FKBP52 and the serin-threonin-phosphotase PP5 should be mentioned (Buchner, 1999, Trends Biochem. Sci 24, 136-141; Dolinski et al., 1998, Mol. Cell. Biol. 18, 7344-7352; Marsh et al., 1998, Mol. Cell. Biol. 18, 7353-7359; Pratt and Toft, 1997, Endocr. Rev. 18, 306-360). Respective experiments by Owens-Grillo et al. (1996, J. Biol. Chem. 271, 13468-13575) and Young et al. (1998, J. Biol. Chem. 273, 18007-18010) suggested that only one TPR acceptor location is present in this region. From the paper by Ballinger et al. (1999, Mol. Cell. Biol. 19, 4535-4545) it can be gathered that, just like the TPR1 domain of Hop proteins, the TPR protein CHIP binds to the C-terminal of Hsp70.

Multiple sequence comparisons of TPR domains of different proteins reveal that there are no strictly conserved amino acid residues in the TPR structure motifs which have a length of 34 amino acid. Lamb et al. (1995, Trends Biochem. Sci. 20, 257-259) found out that there is nevertheless a strong preference for small hydrophobe amino acids at certain positions of the structure motifs.

In addition, a crystal structure of a TPR domain, being the Hsp90-binding protein phosphotase 5 (PP5), however without a (physiological or unphysiological) peptide ligand, was solved, showing that each TPR structure motif forms a helix-turn-helix-motif (Das et al., 1998, EMBO J. 17, 1192-1199). During this process, neighbouring TPR motifs a packed into an organised sequence of antiparallel a-helices. Therefore, Das et al. (1998, EMBO J. 17, 1192-1199) suggested that the TPR domains recognise specific structure elements of higher ranking, eg. secondary or tertiary structures, in order to fulfil their biological function. From the publications and understanding of prior art it is, however, not possible to determine the structural basis of the interaction between proteins with TPR domains and, for example, chaperone proteins, such as Hsp70 and/or Hsp90.

Thus, it is the object of the present invention, to obtain an understanding of the interaction between TPR domains and their binding partners on the basis of bio-physical methods, and to apply this understanding, for example, for the modelling of inhibitors, which have, in form of molecular mimicry, a high binding ability to the TPR domain, or alternatively to the chaperone protein, thus competitively blocking the biological function of the physiological binding partners.

Thus, according to the invention, crystal structures of TPR domains of the Hop protein (TPR1, TPR2A) are disclosed, in the presence and absence of peptides, which simulate the binding behaviour of Hop and chaperone proteins. These structures allow assumptions to be made regarding the kind of interaction between TPR domains containing co-chaperones and chaperone proteins, such as Hsp70 and Hsp90.

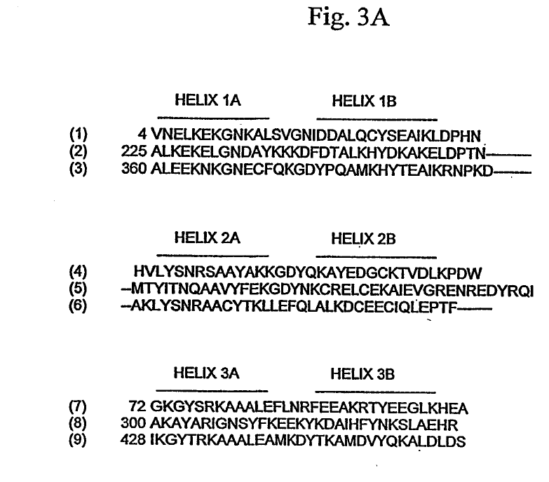

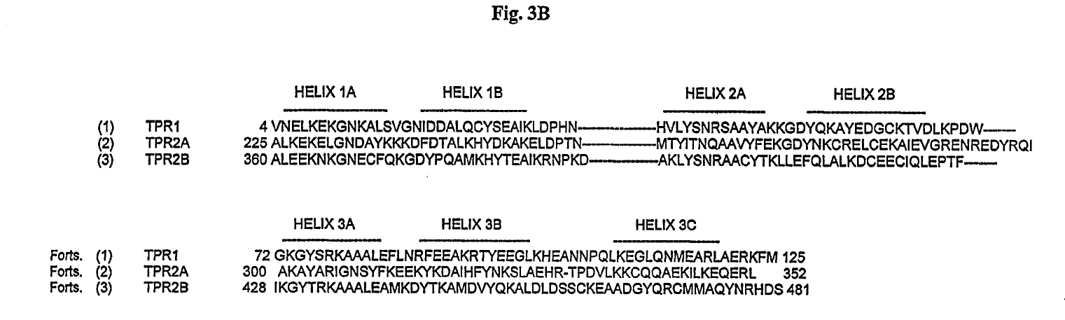

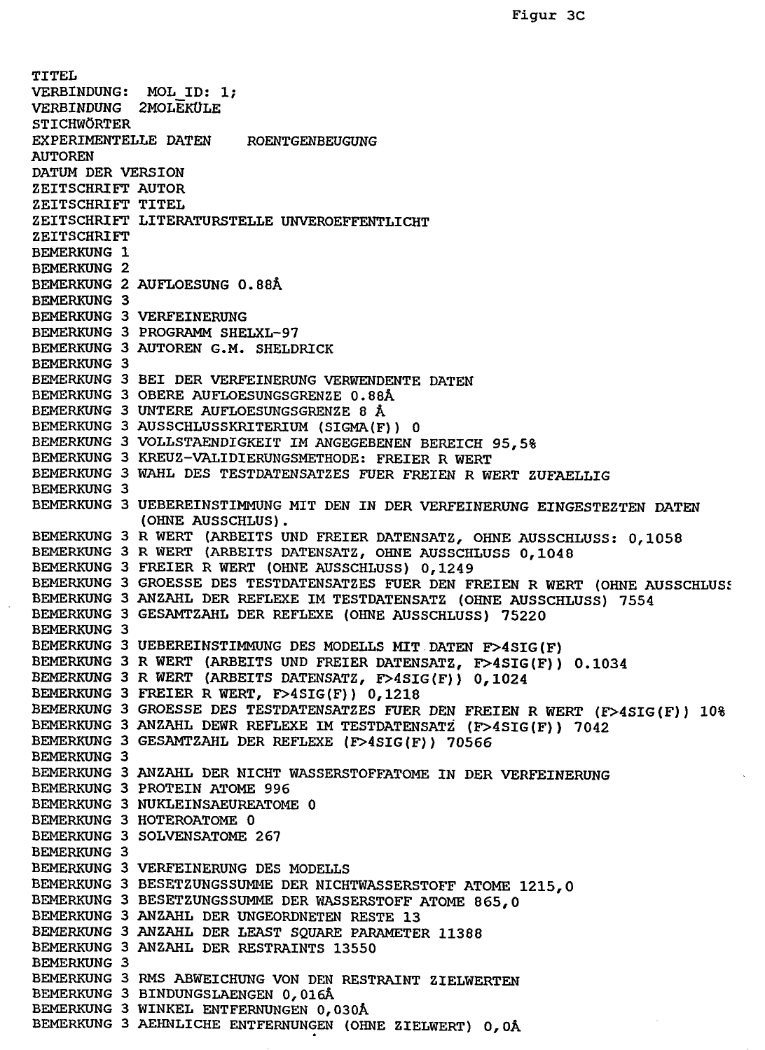

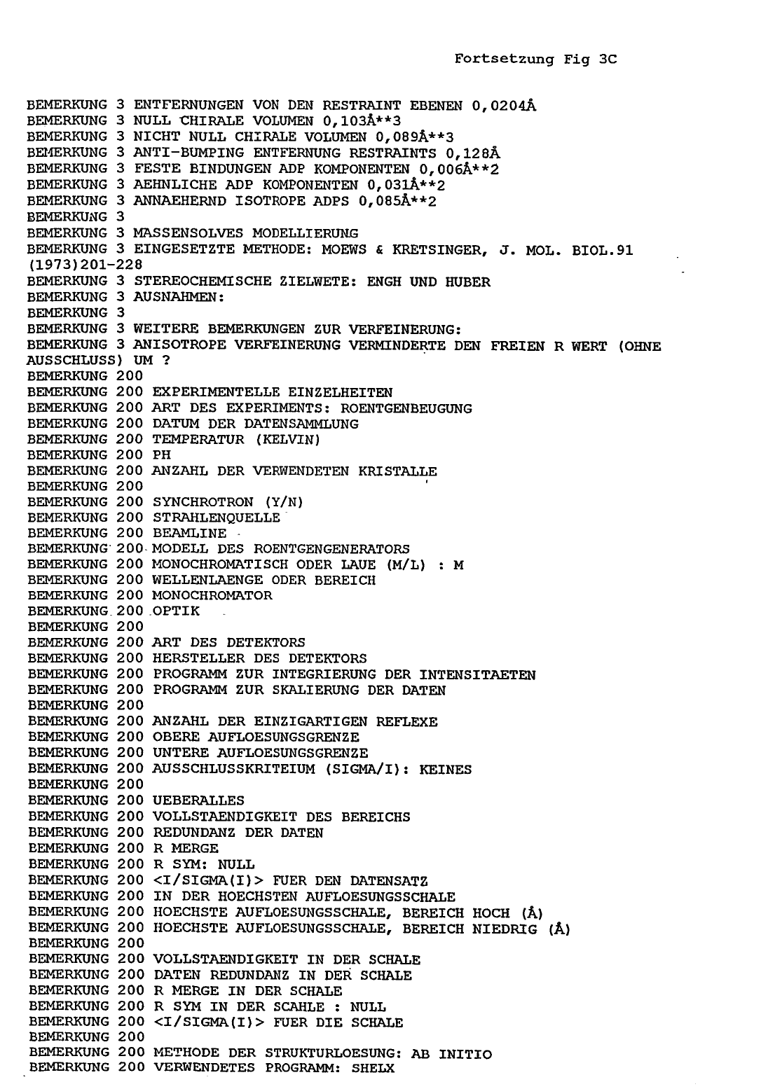

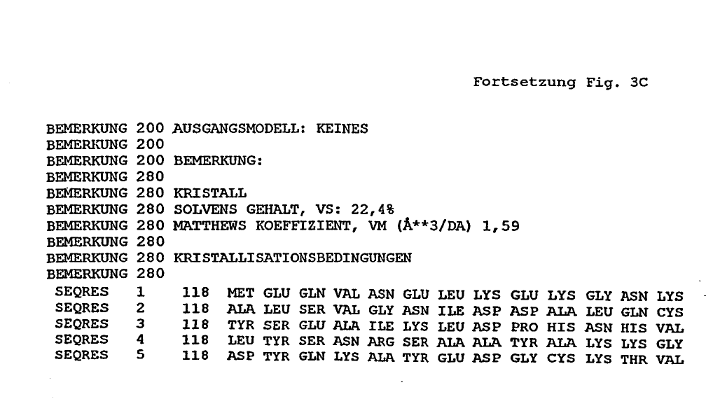

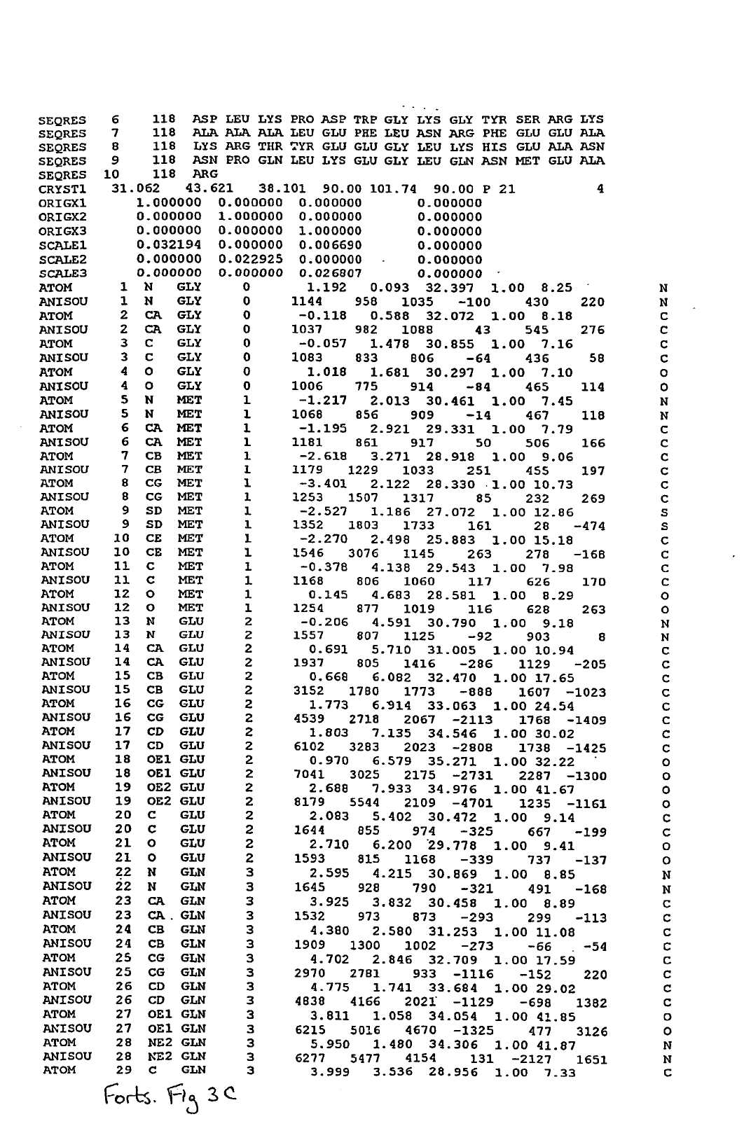

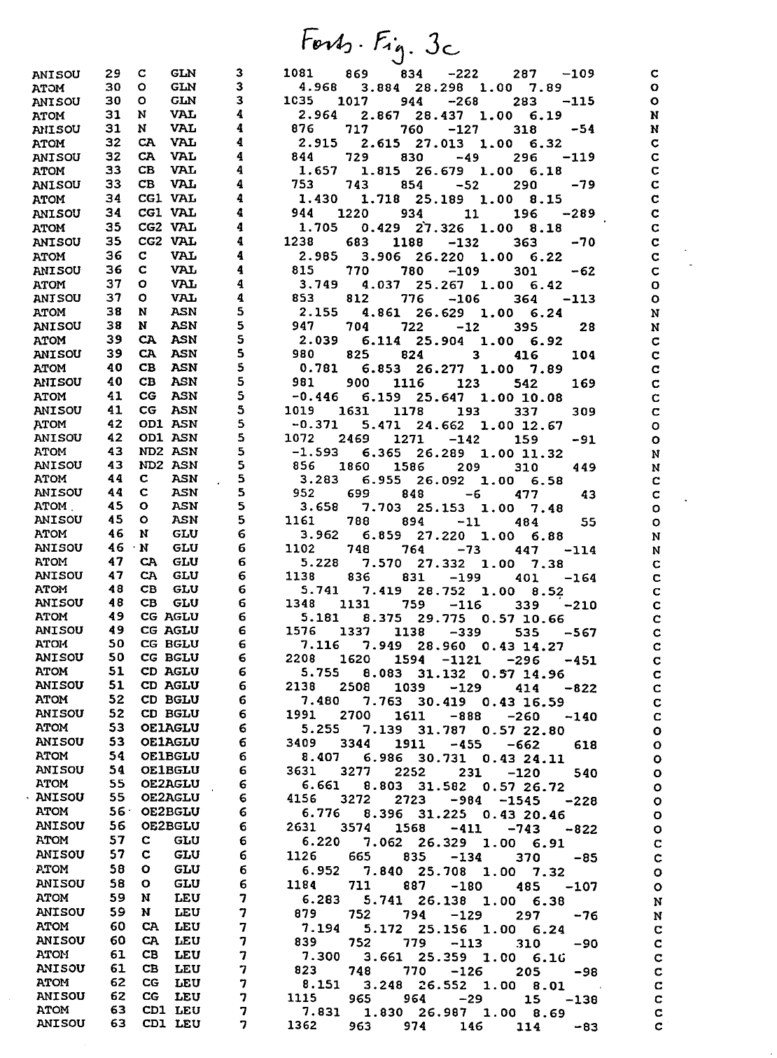

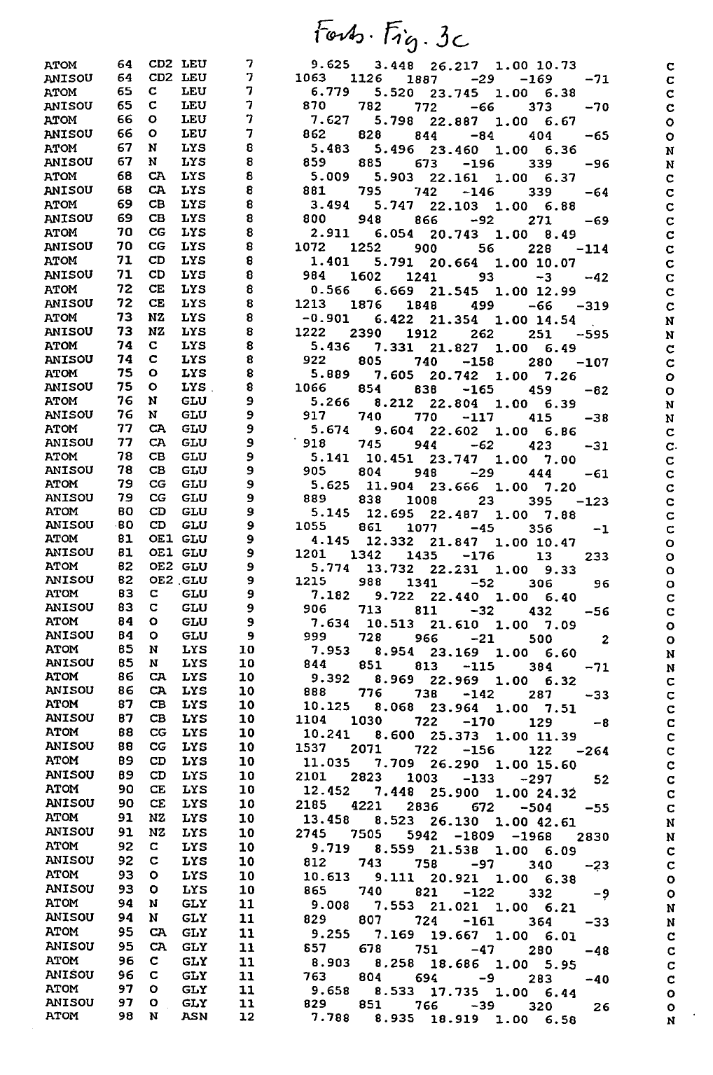

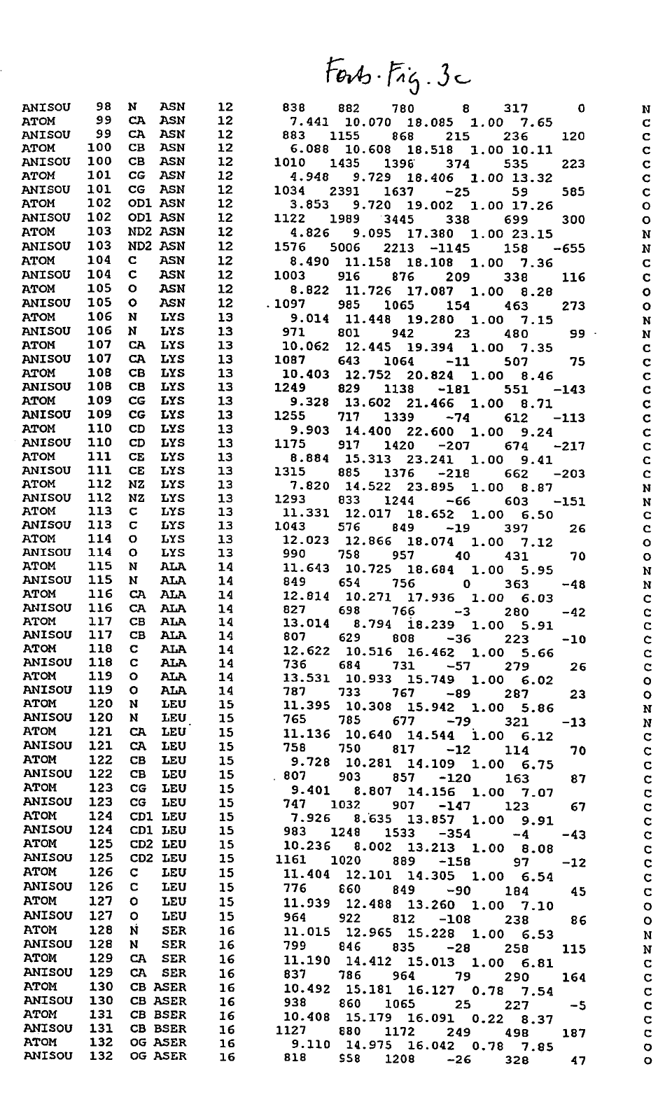

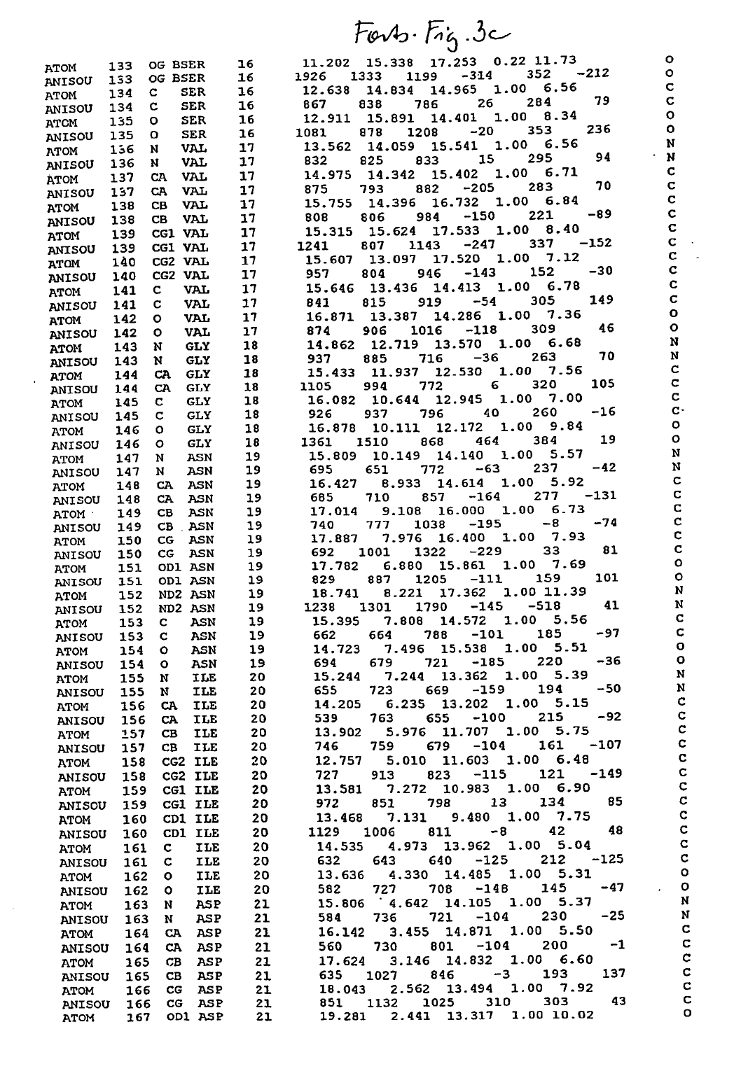

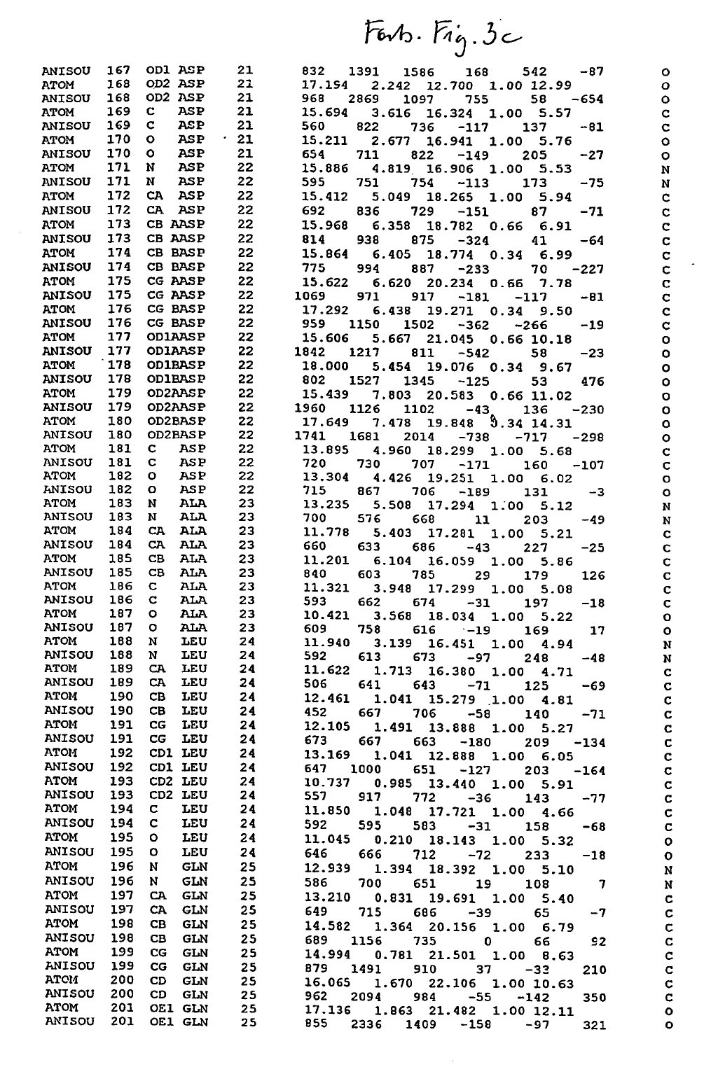

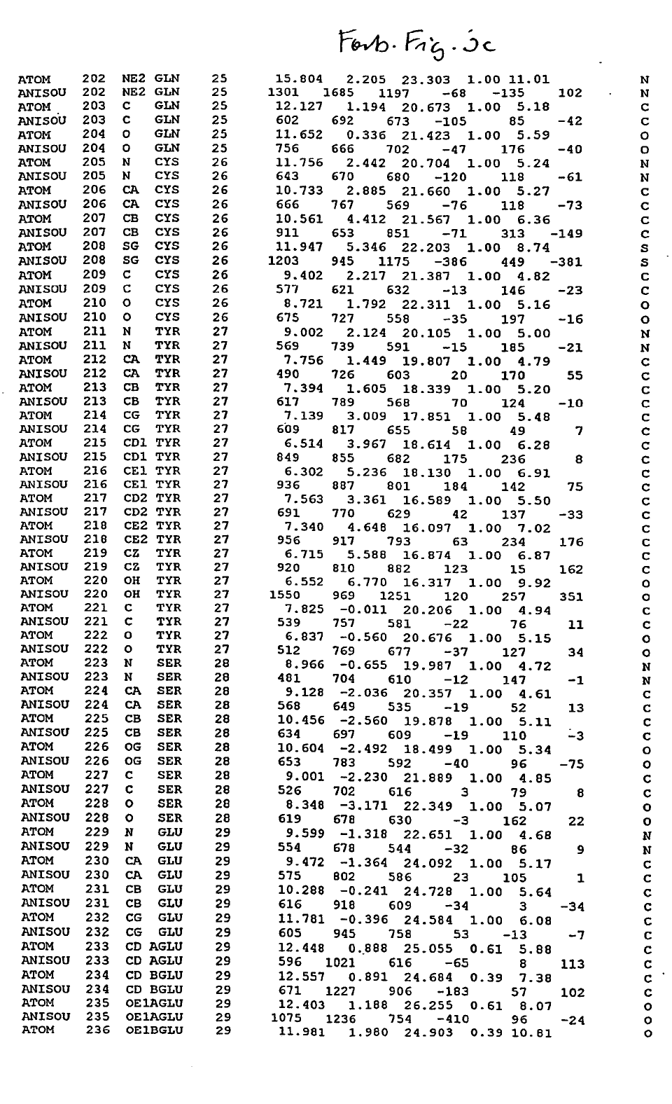

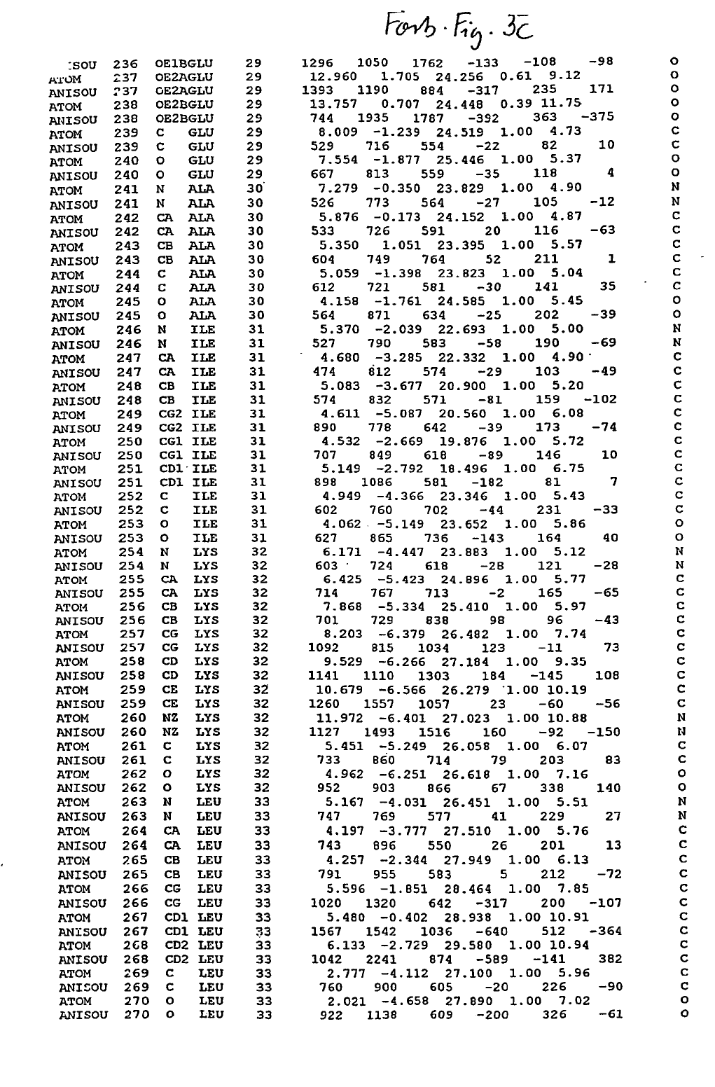

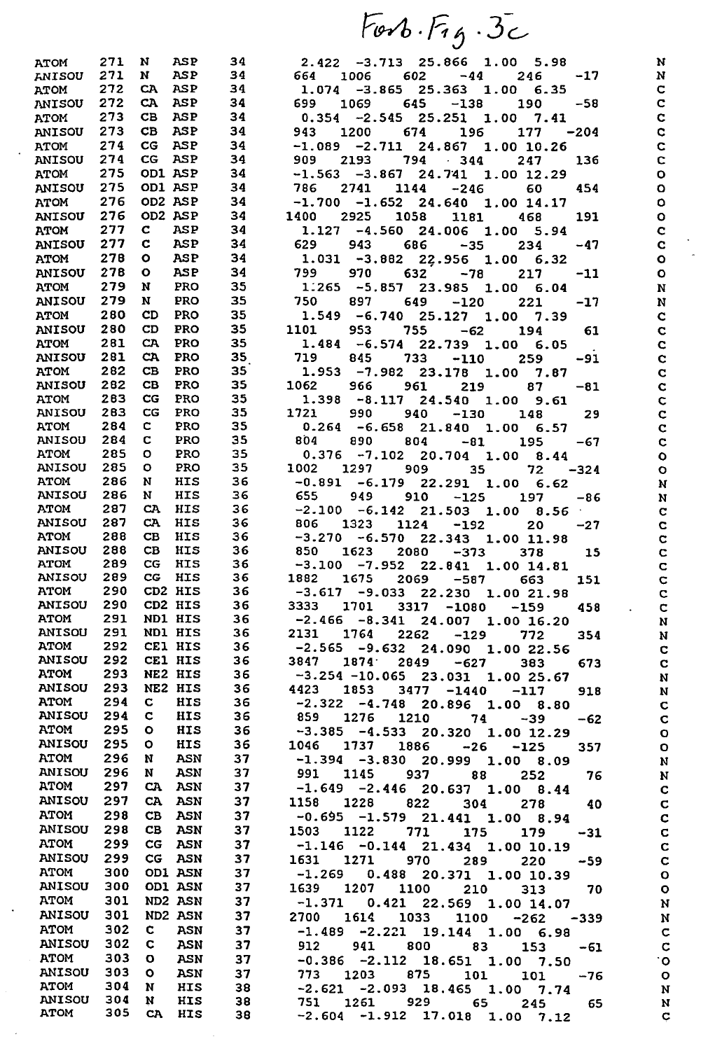

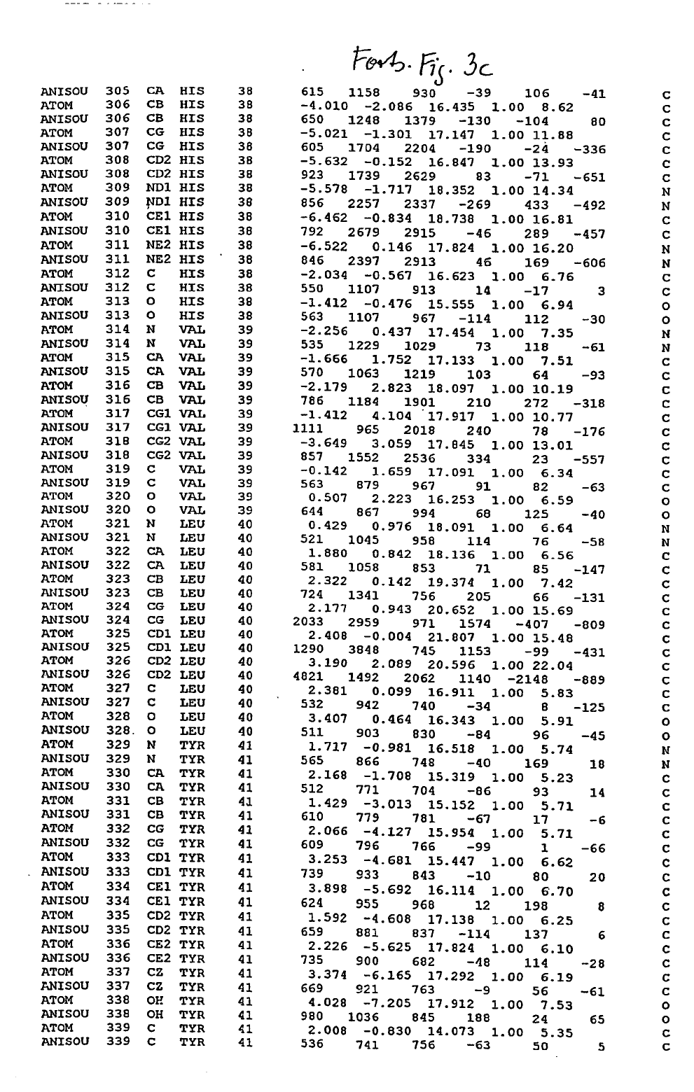

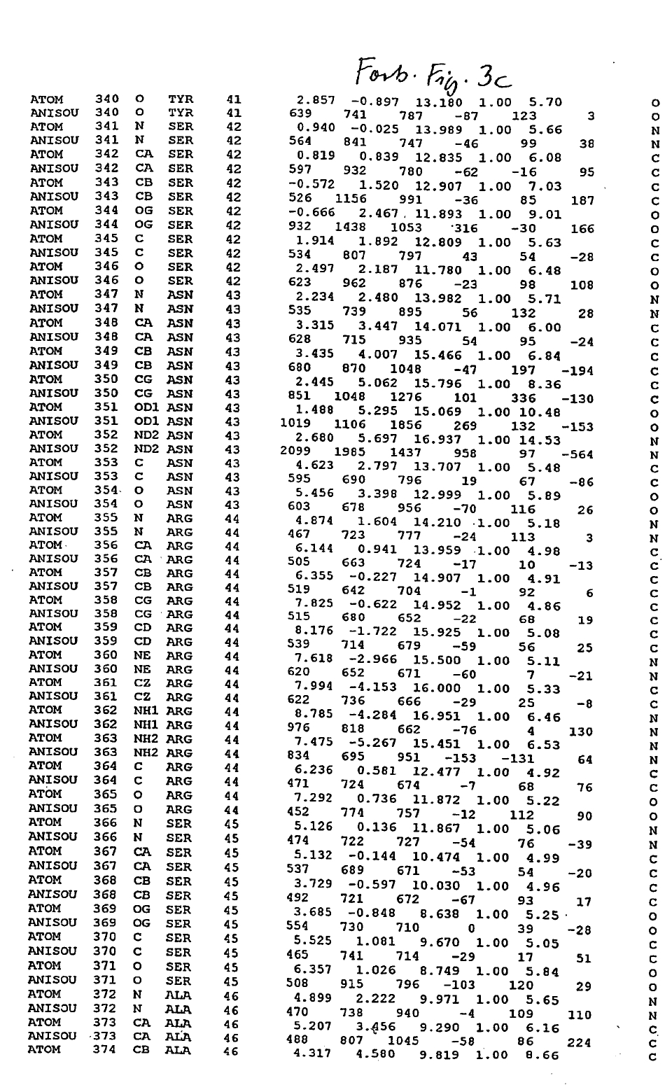

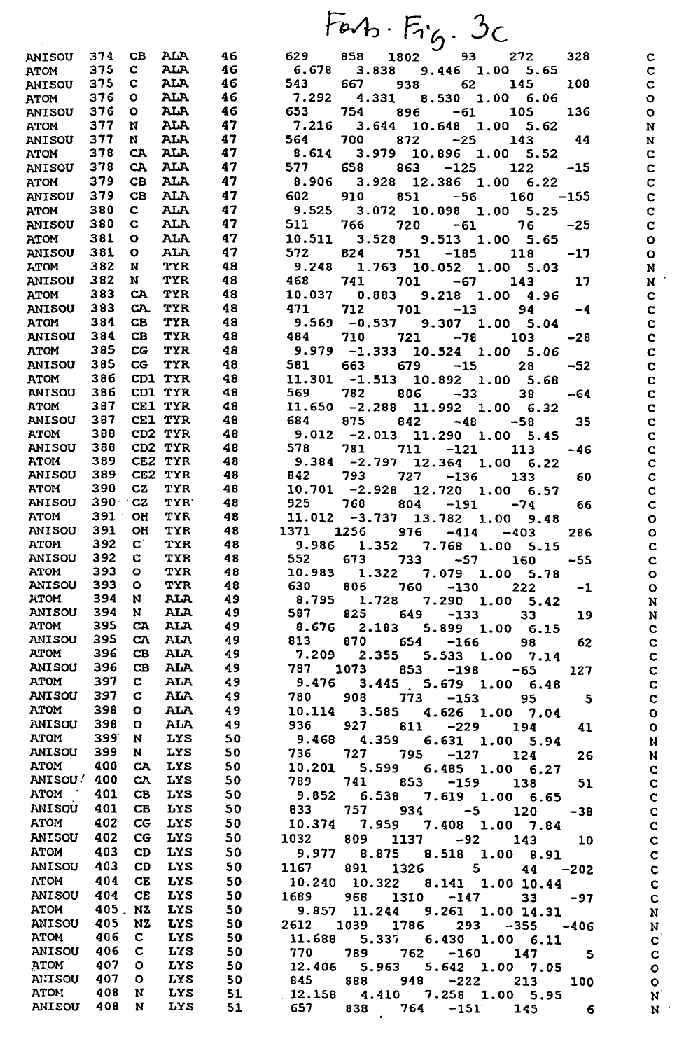

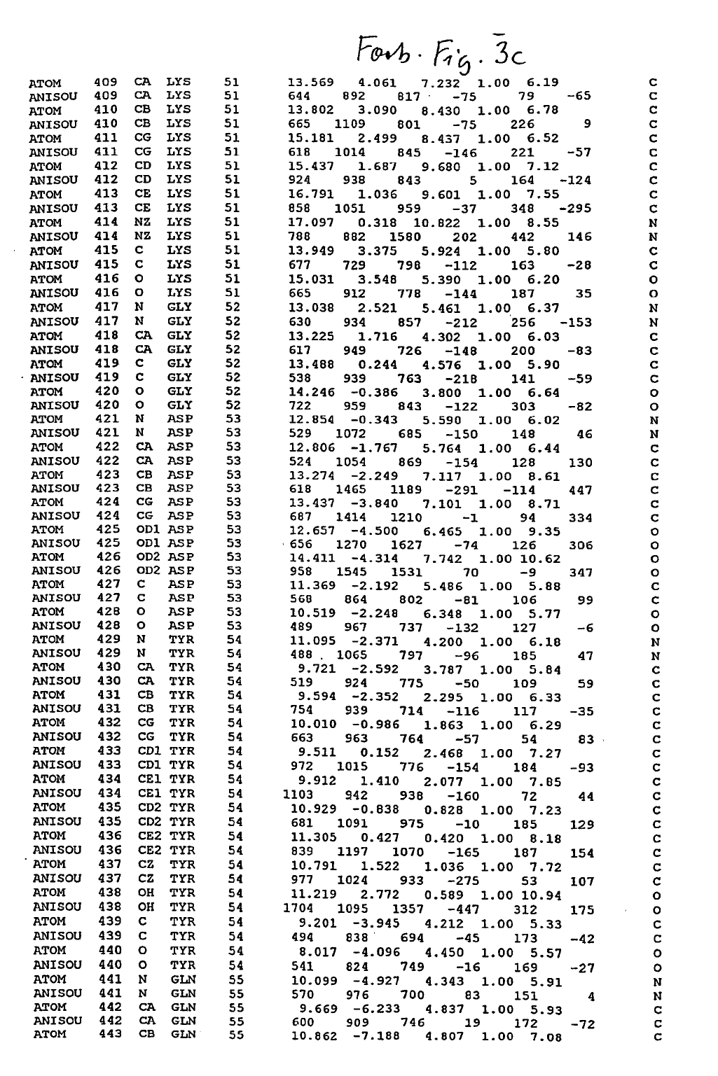

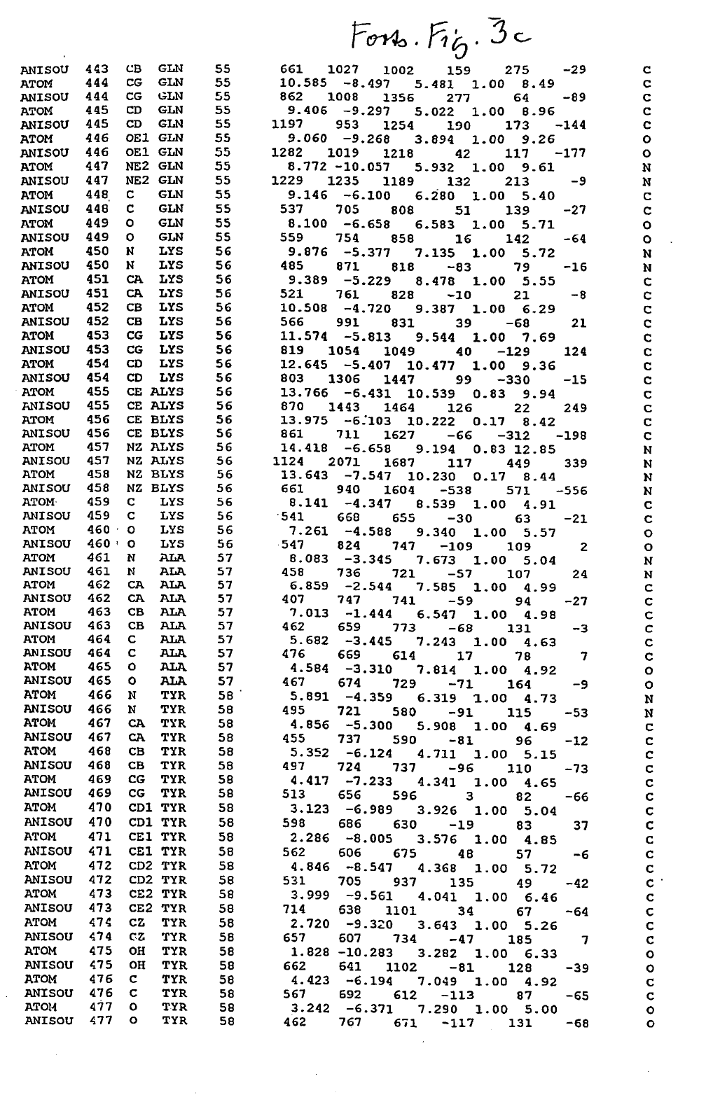

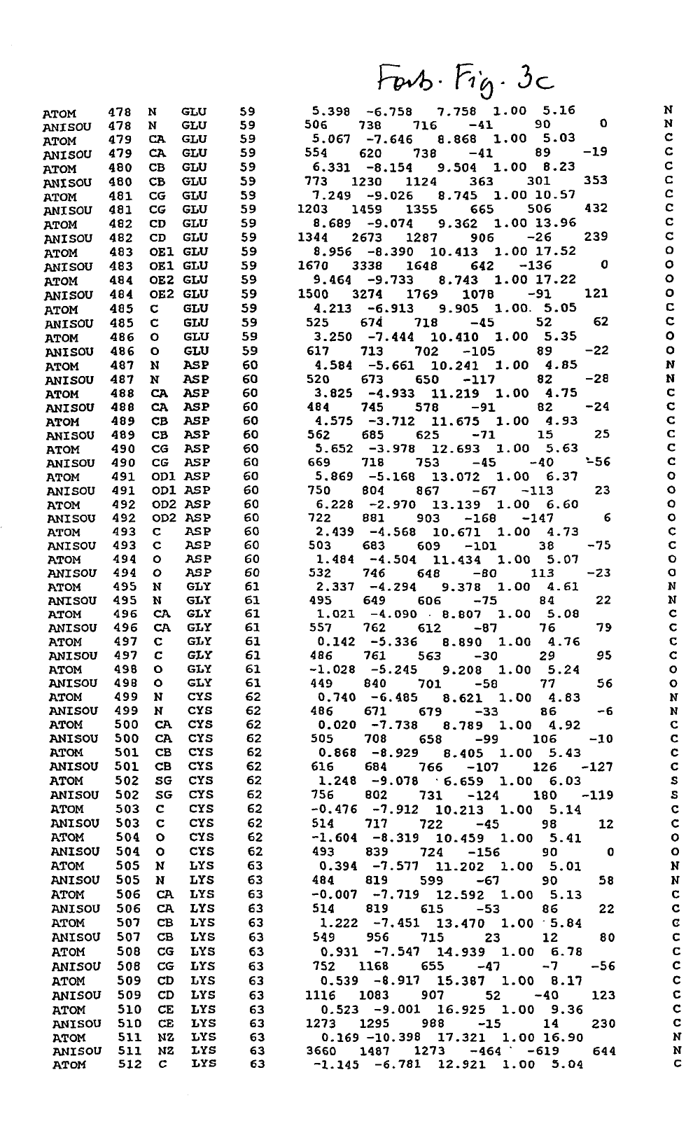

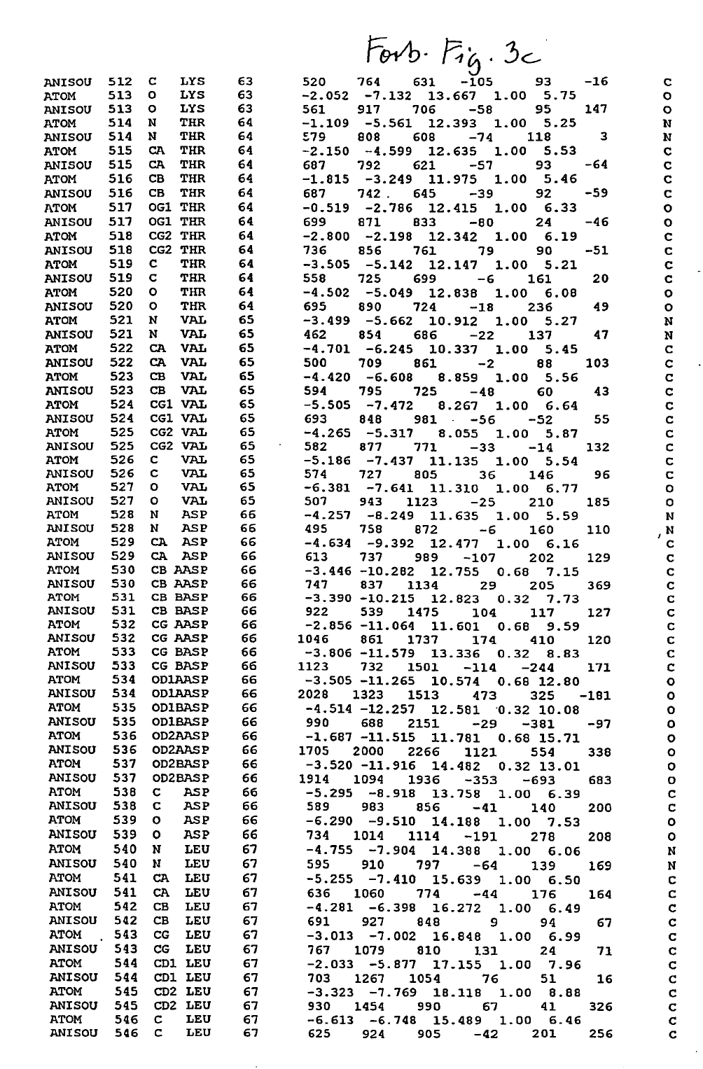

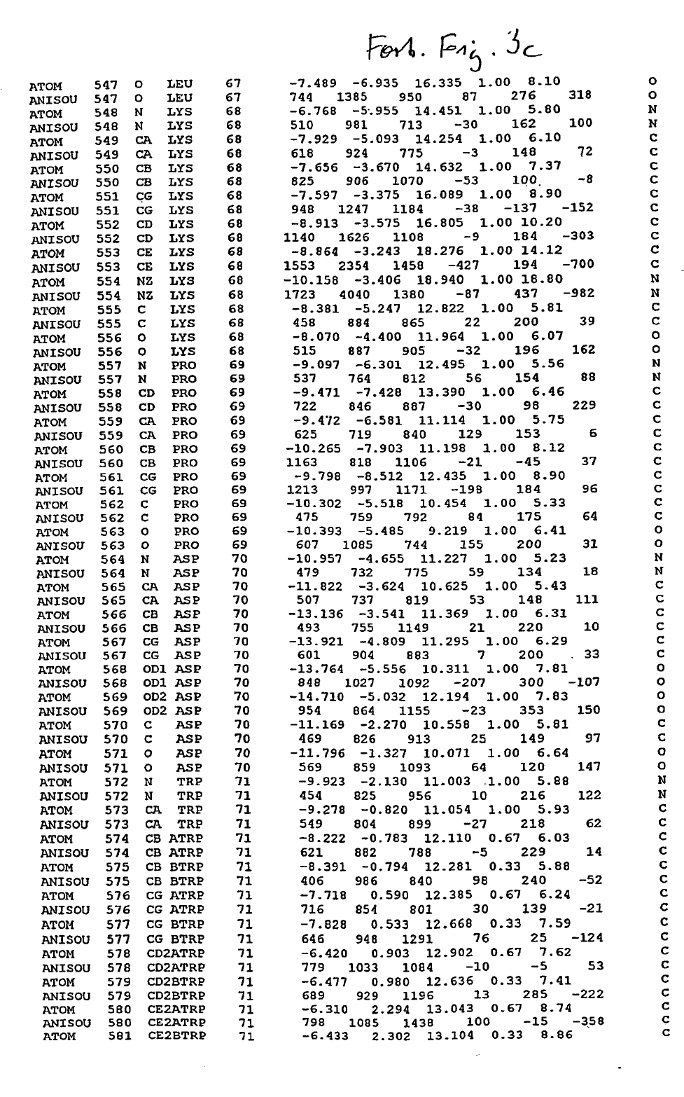

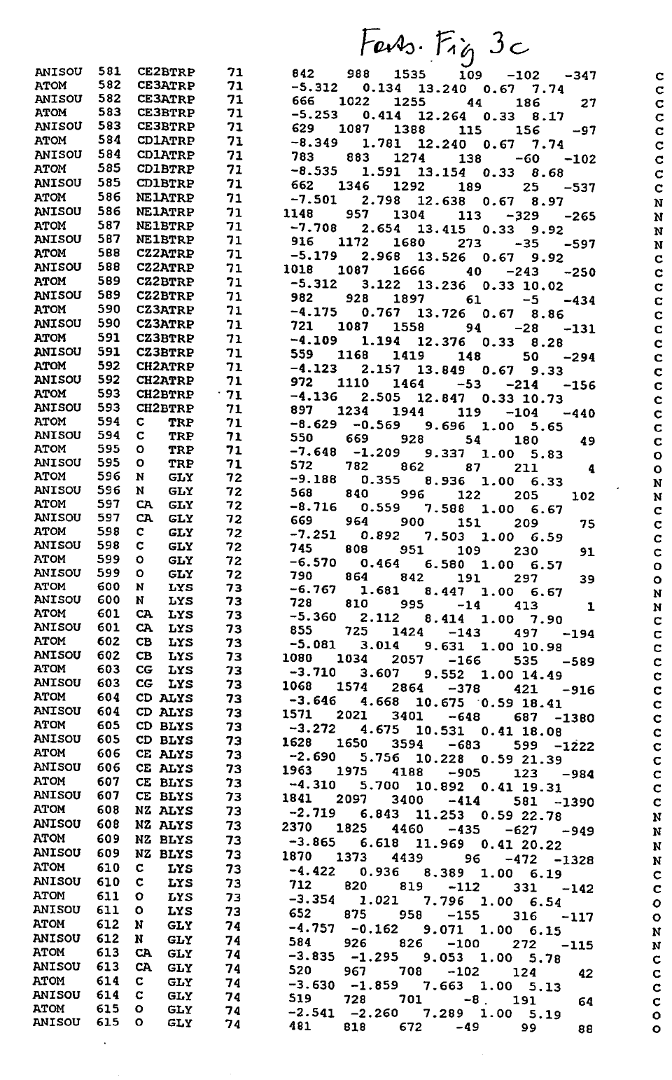

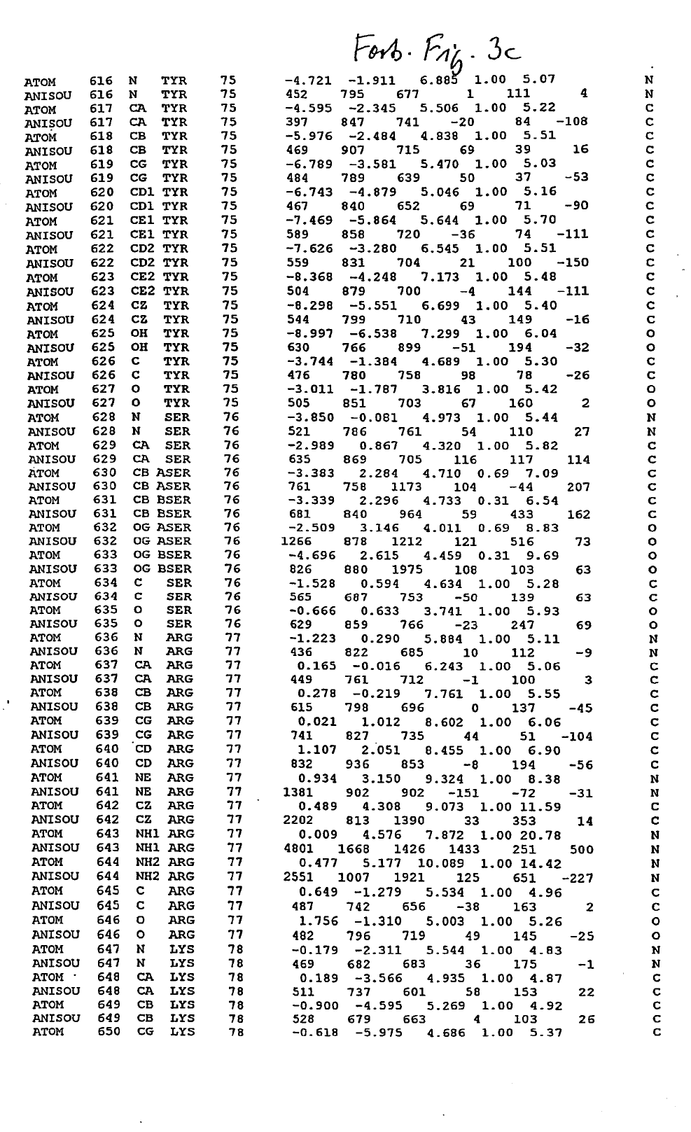

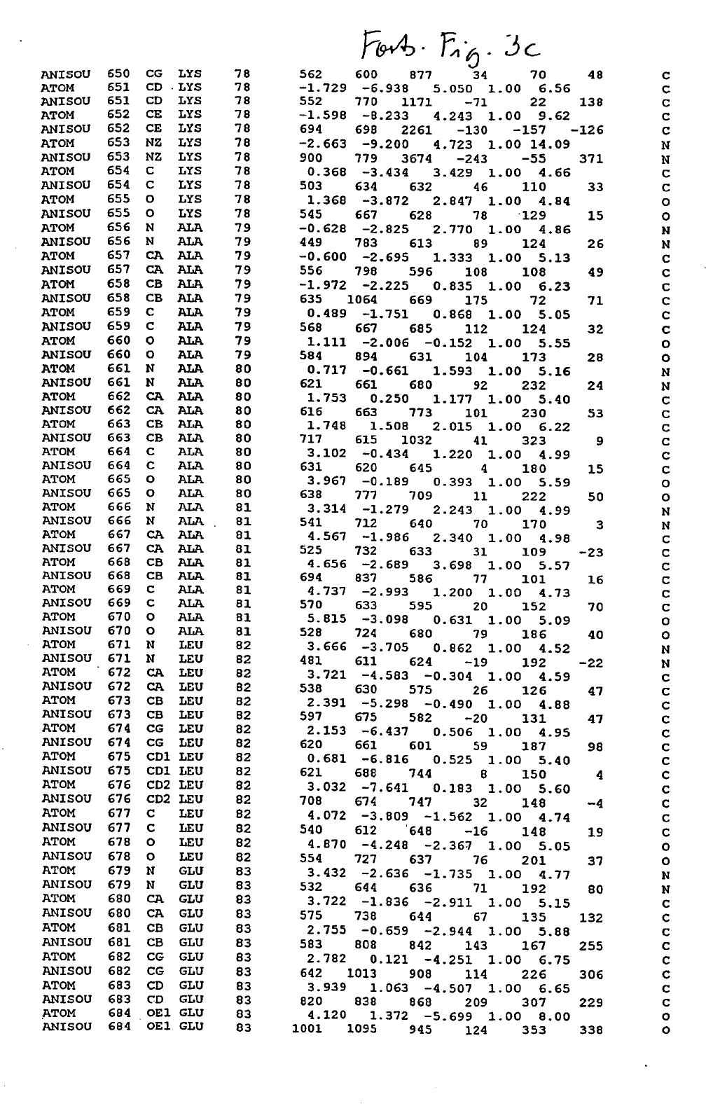

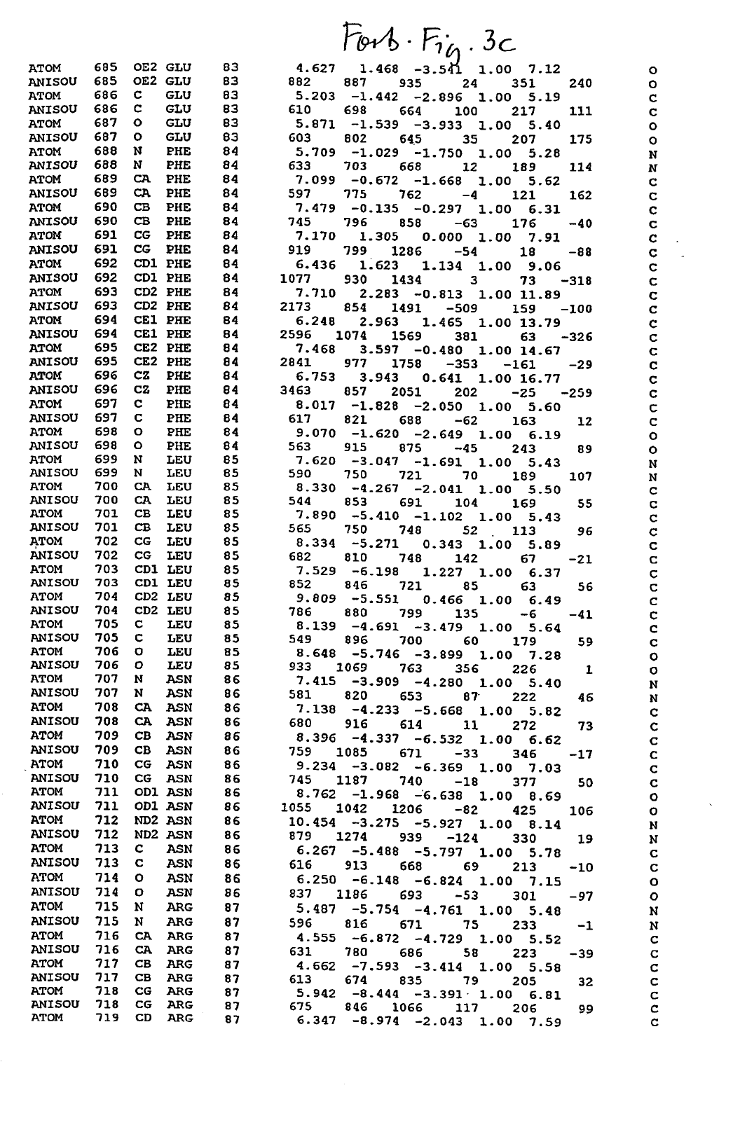

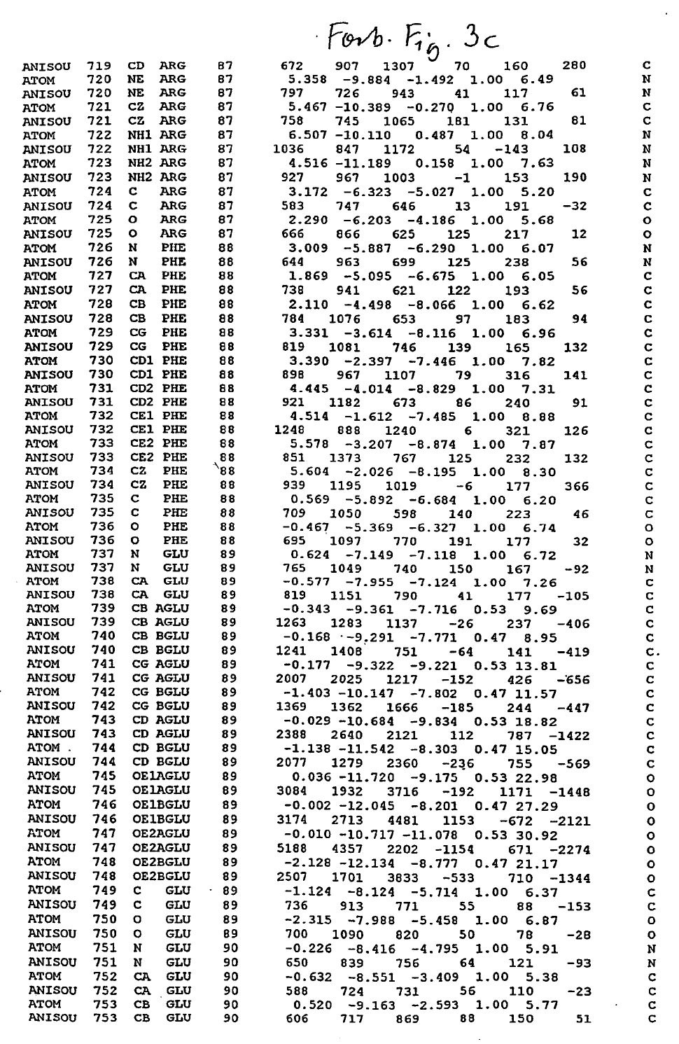

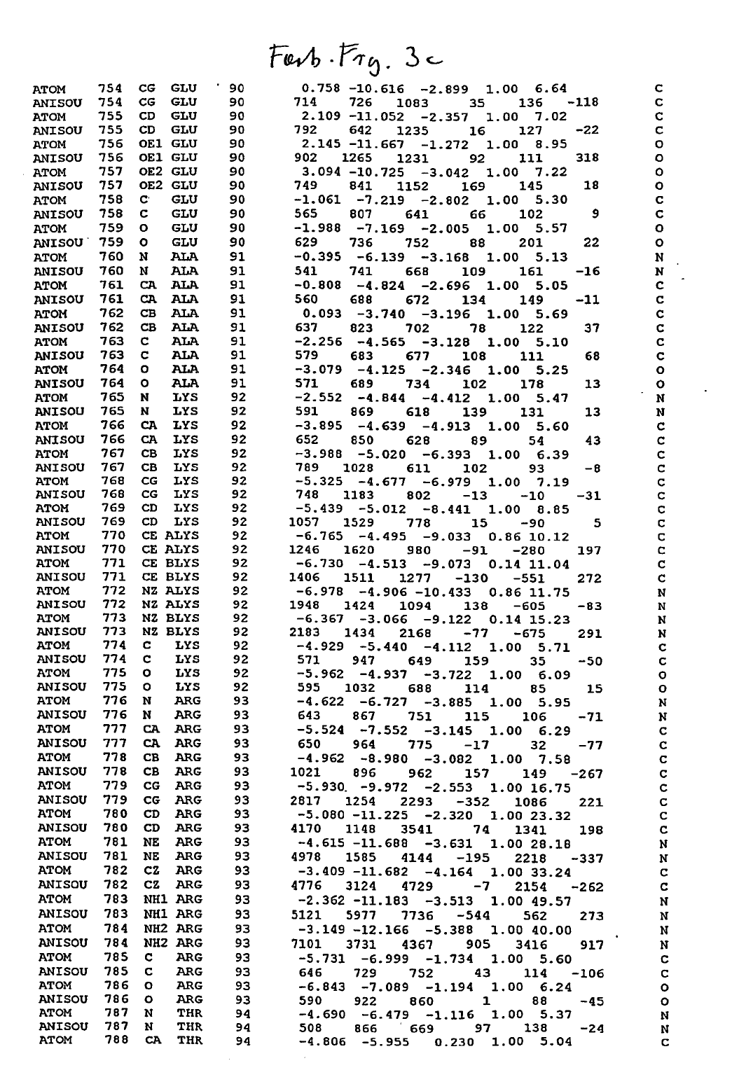

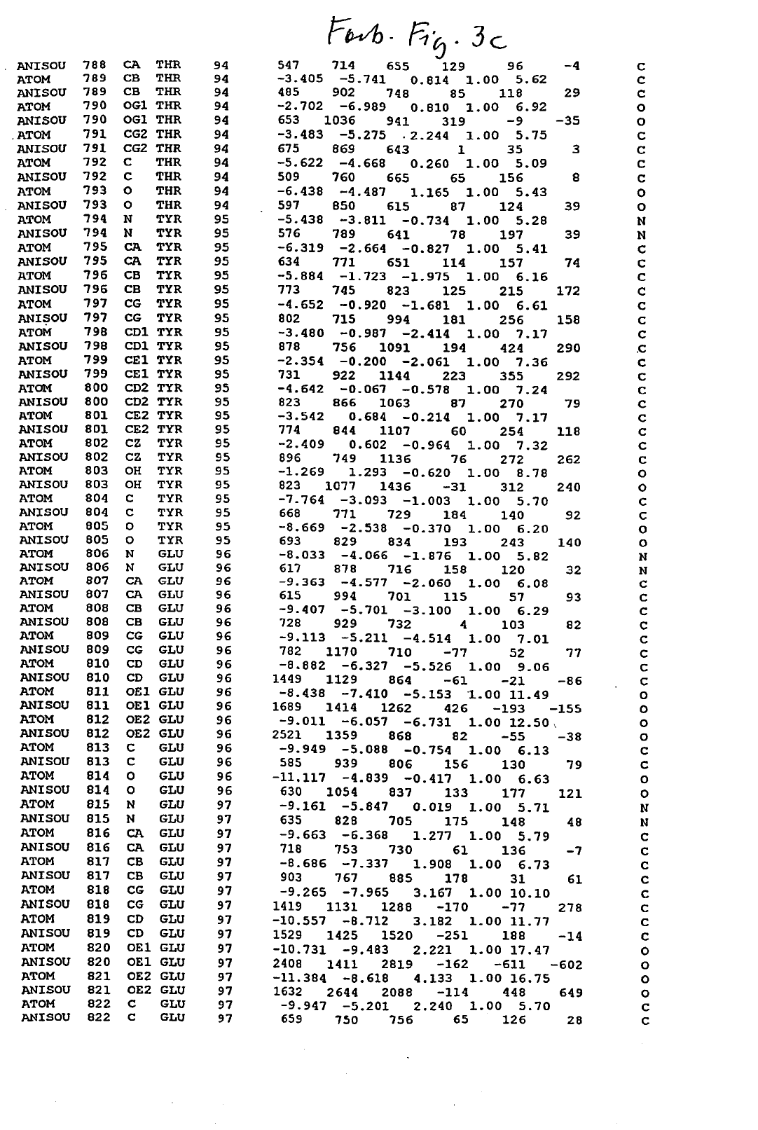

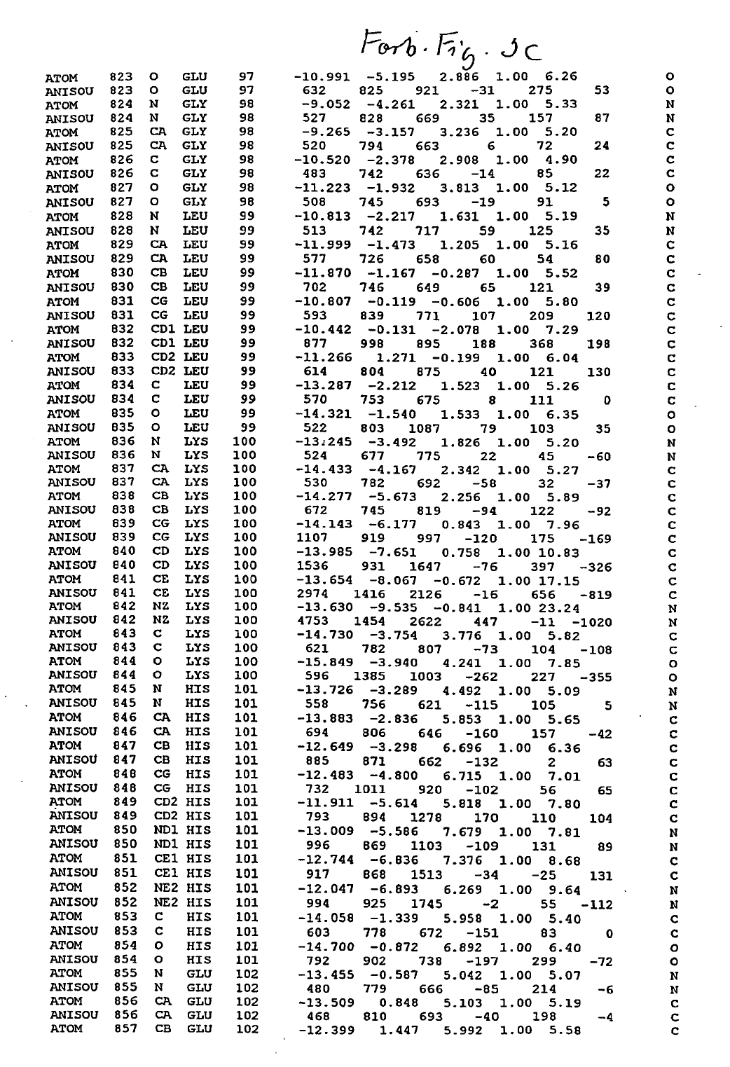

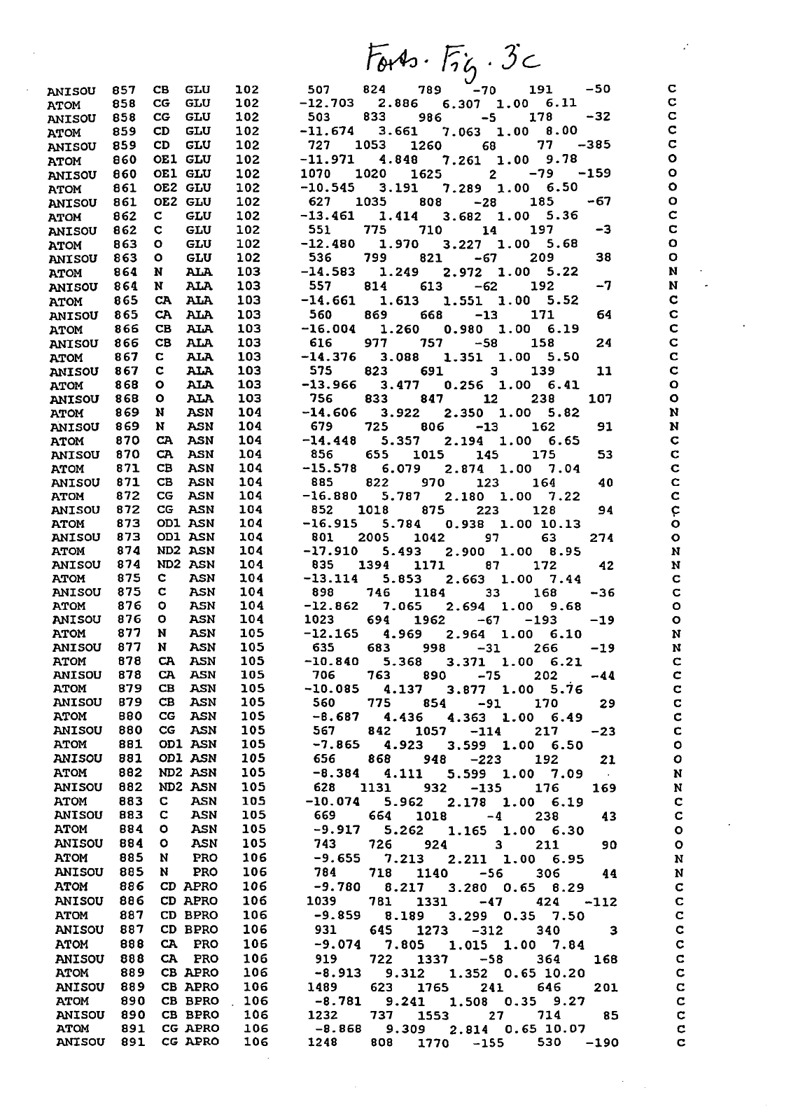

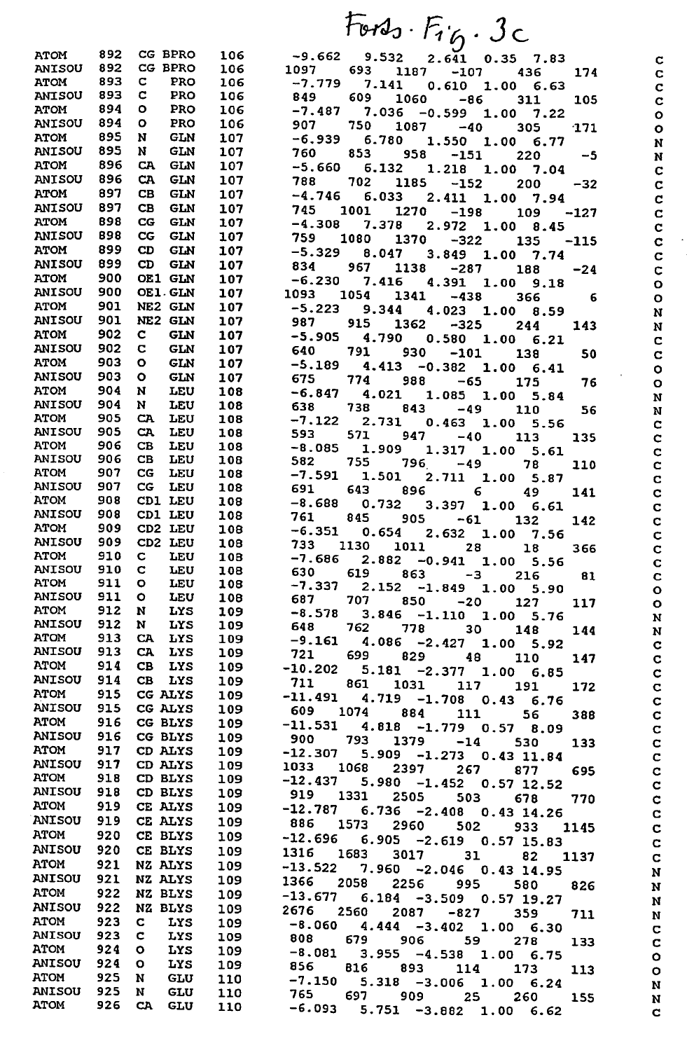

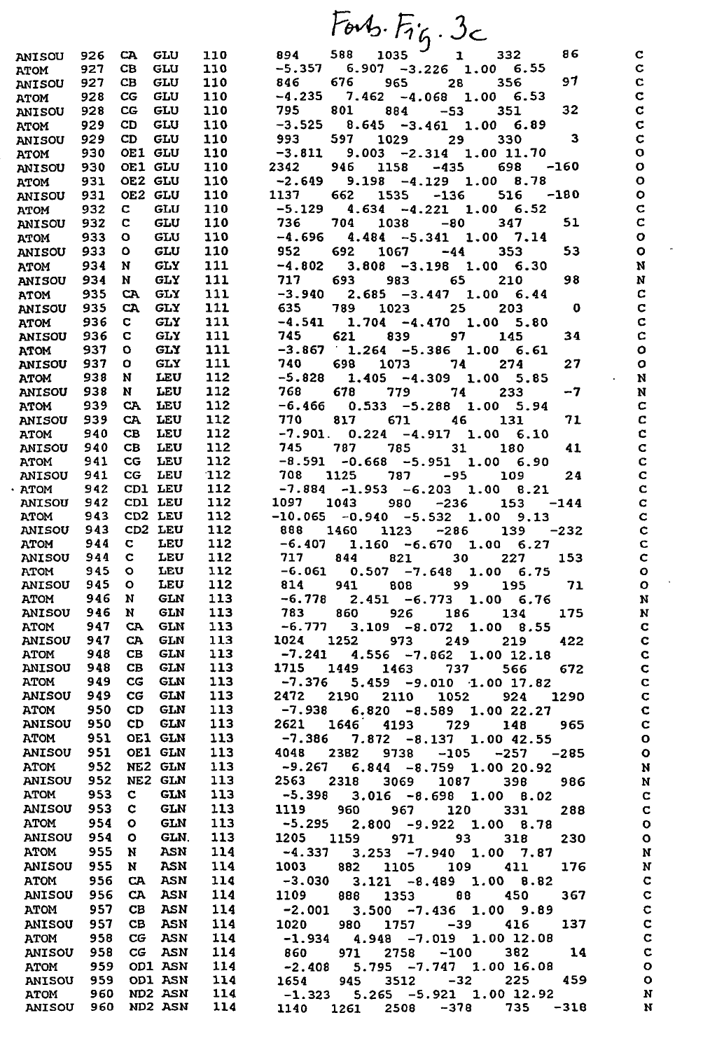

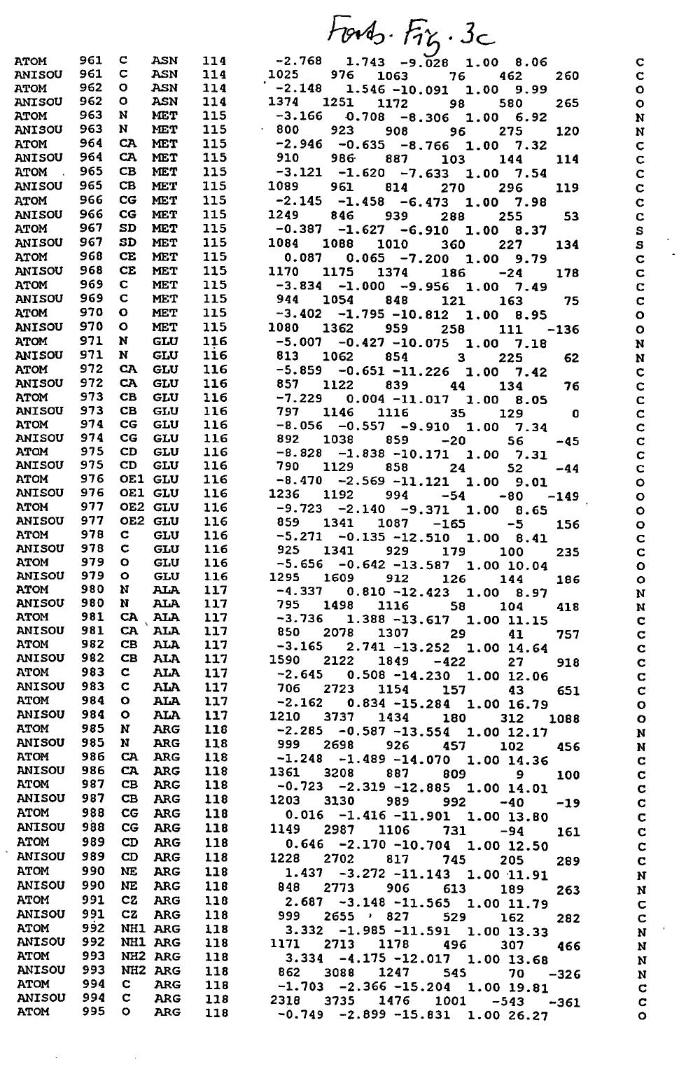

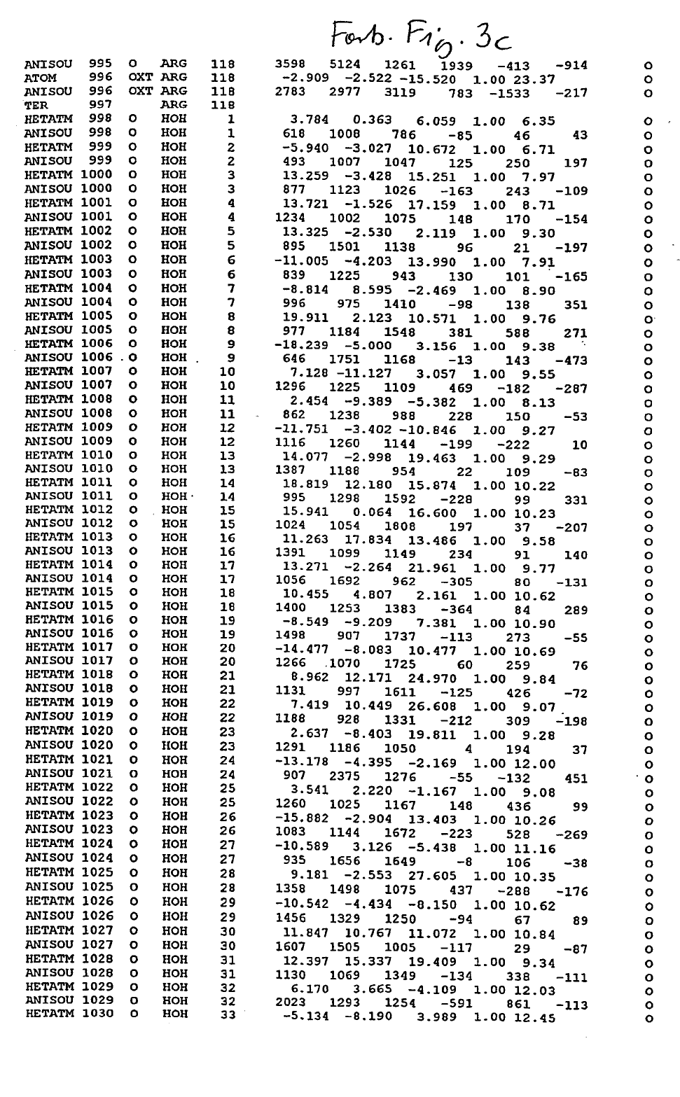

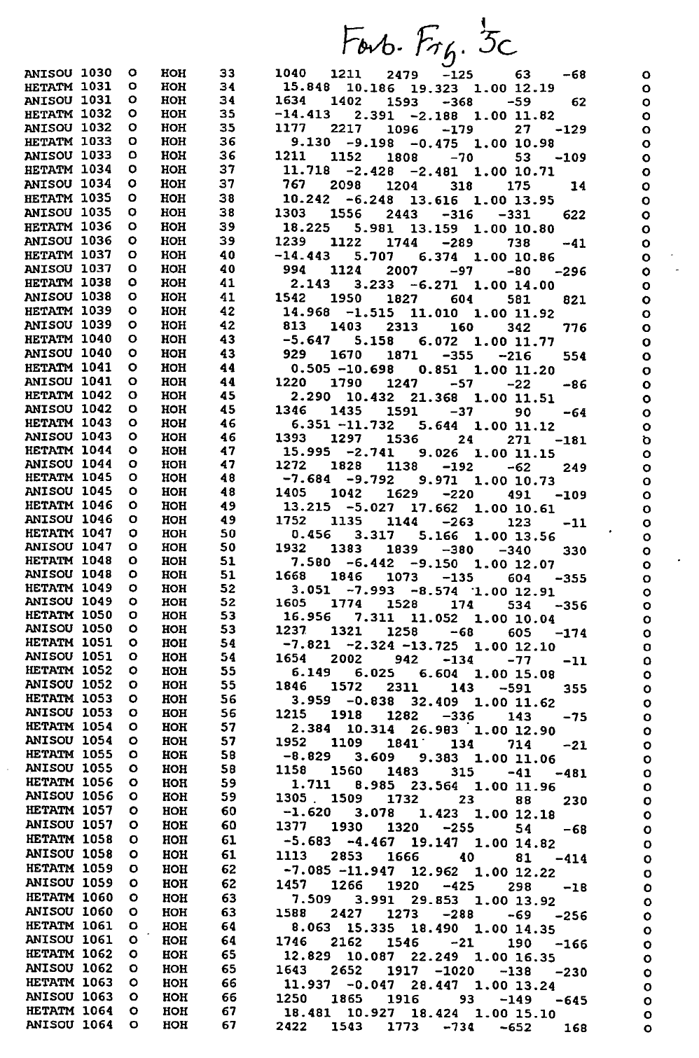

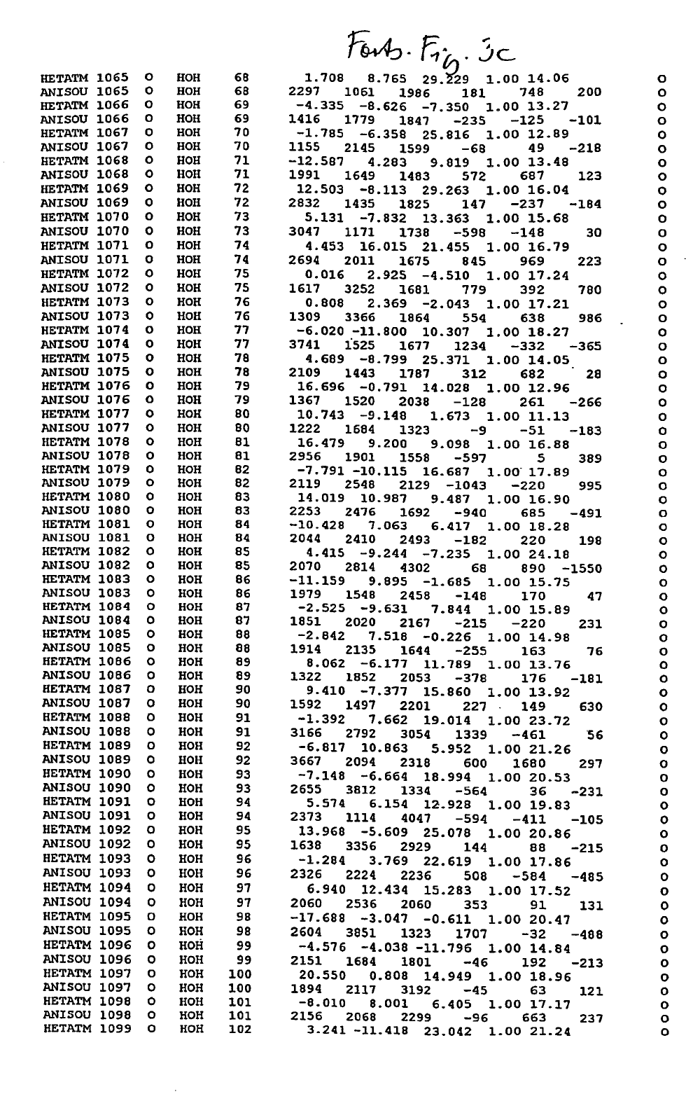

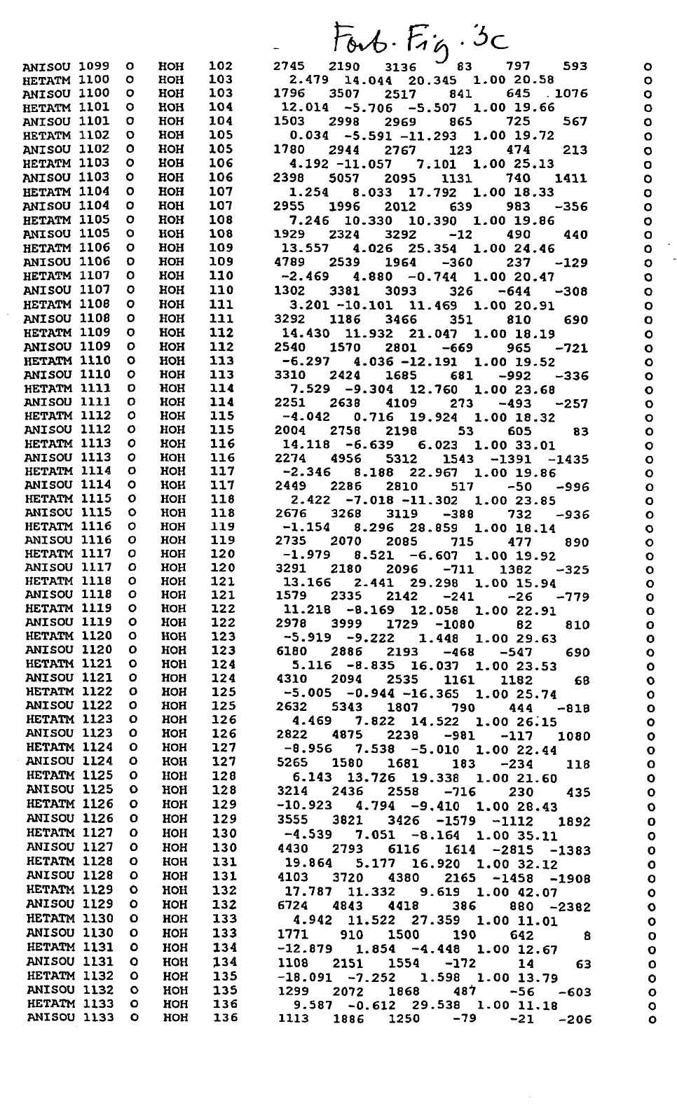

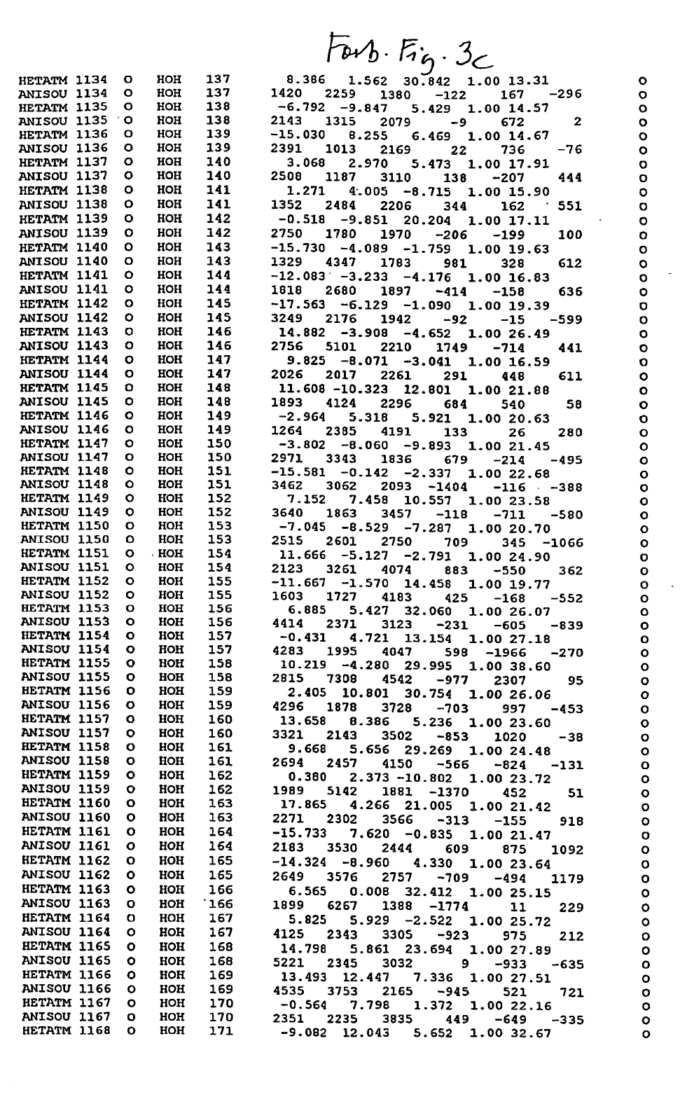

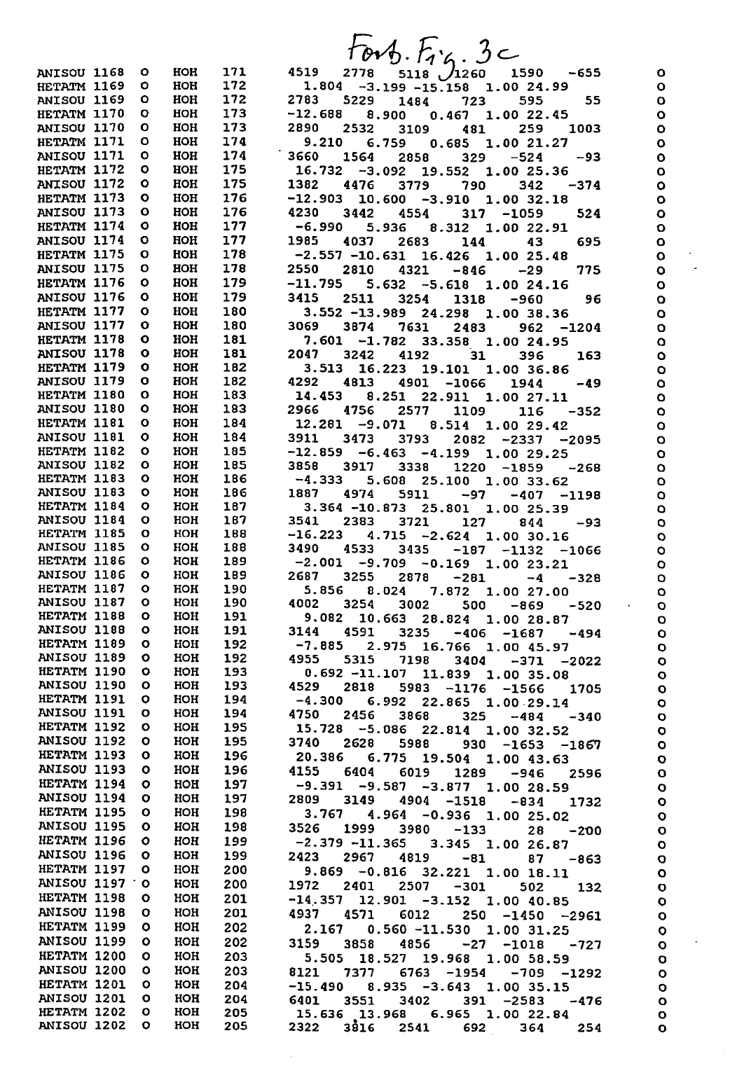

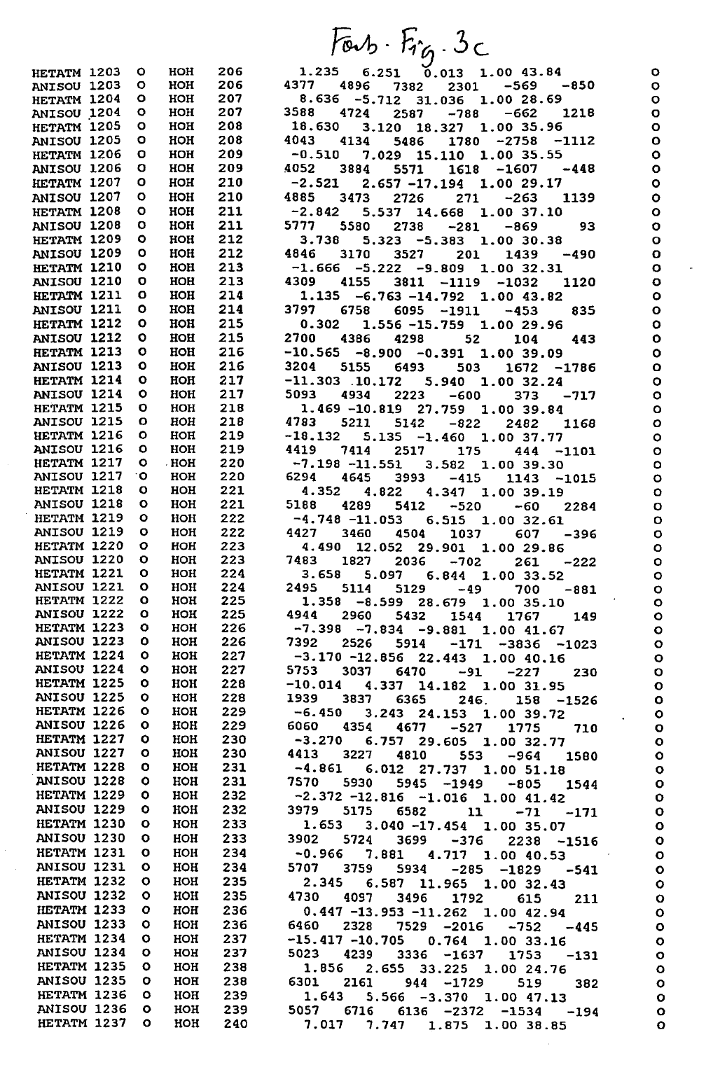

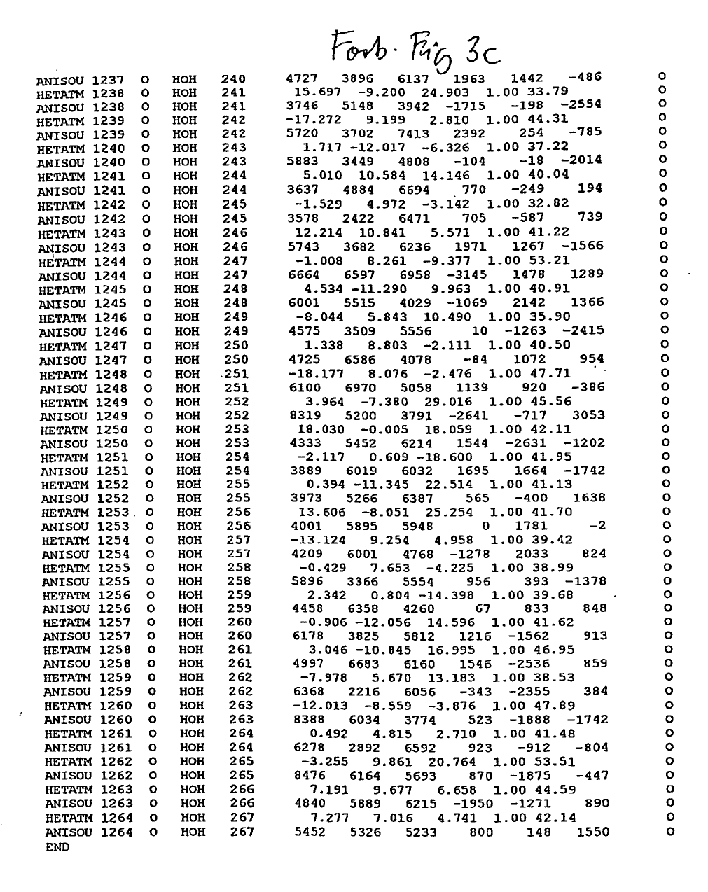

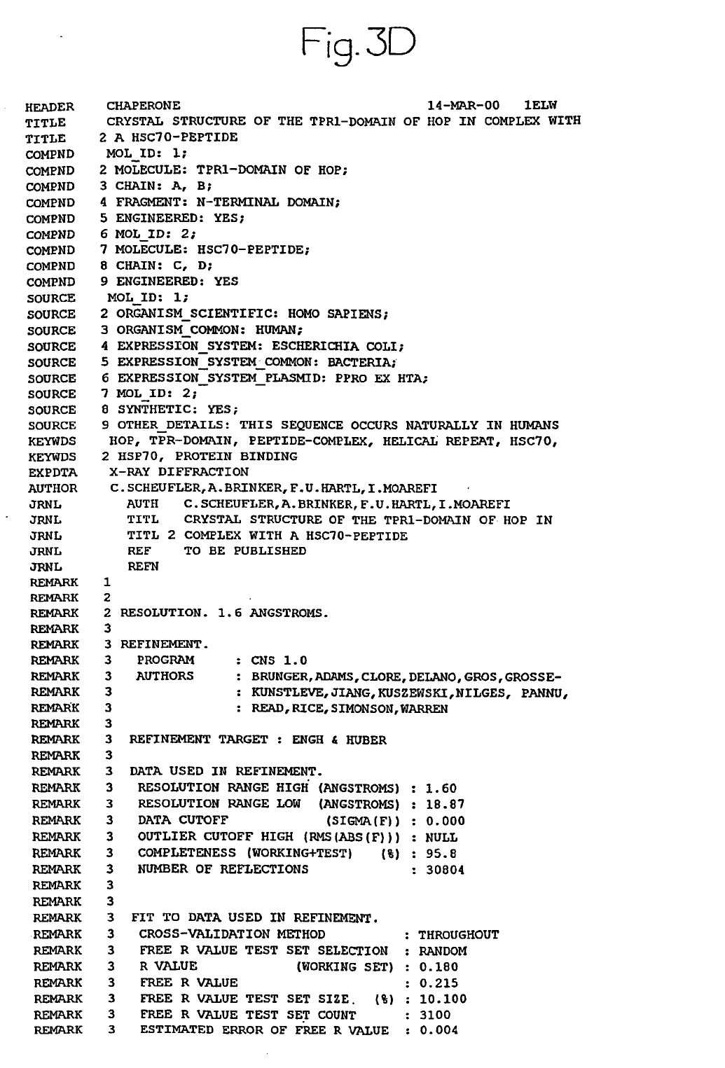

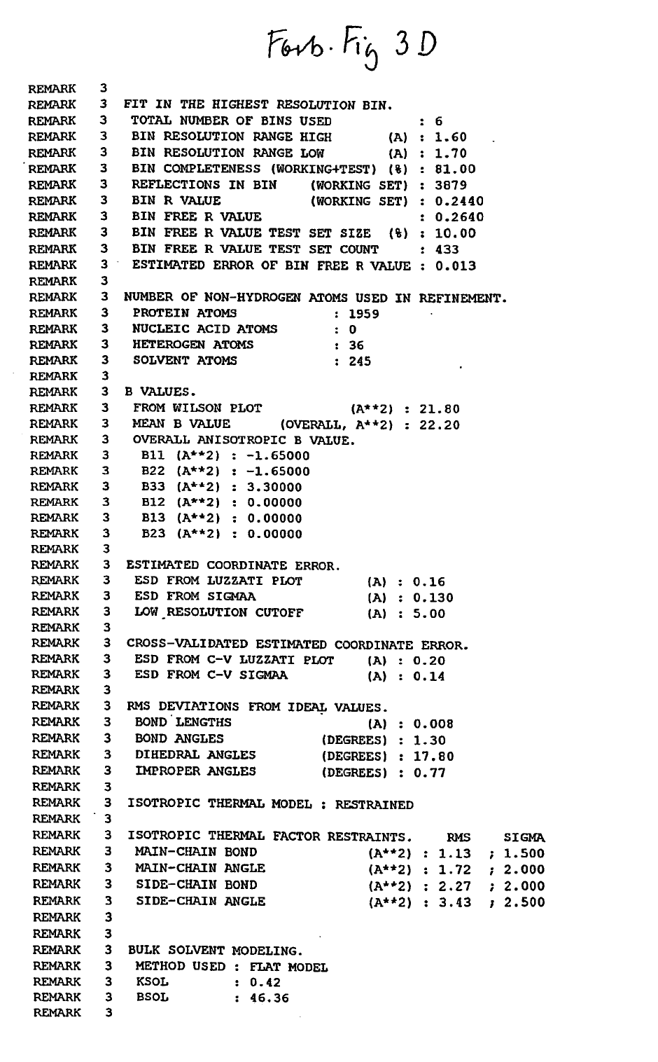

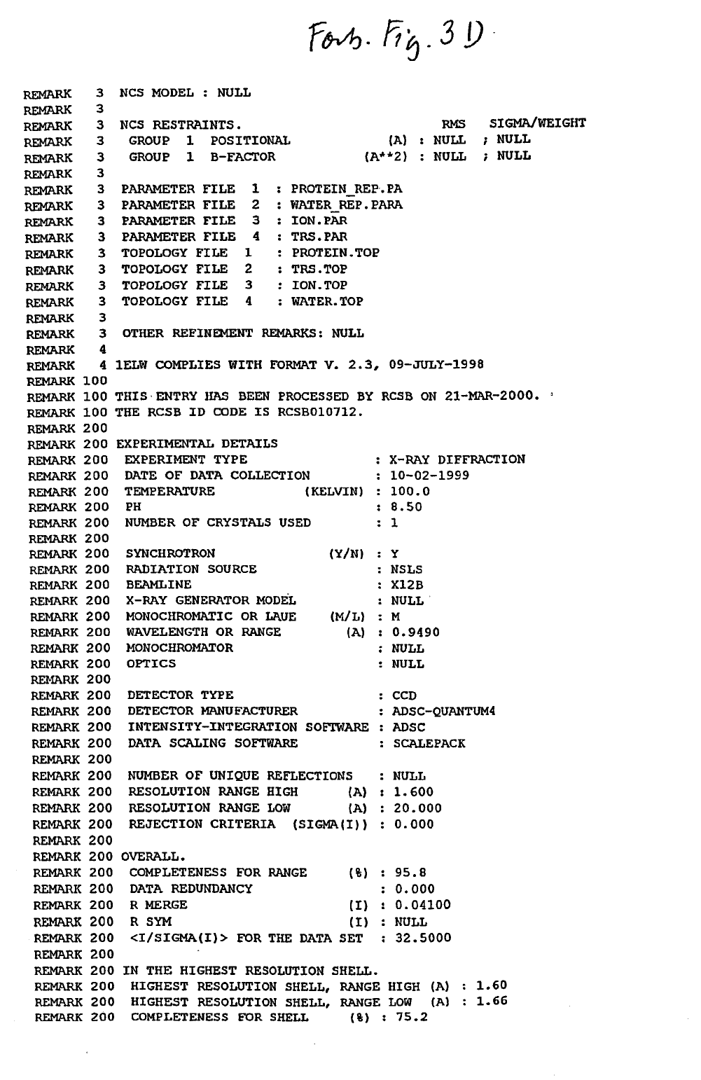

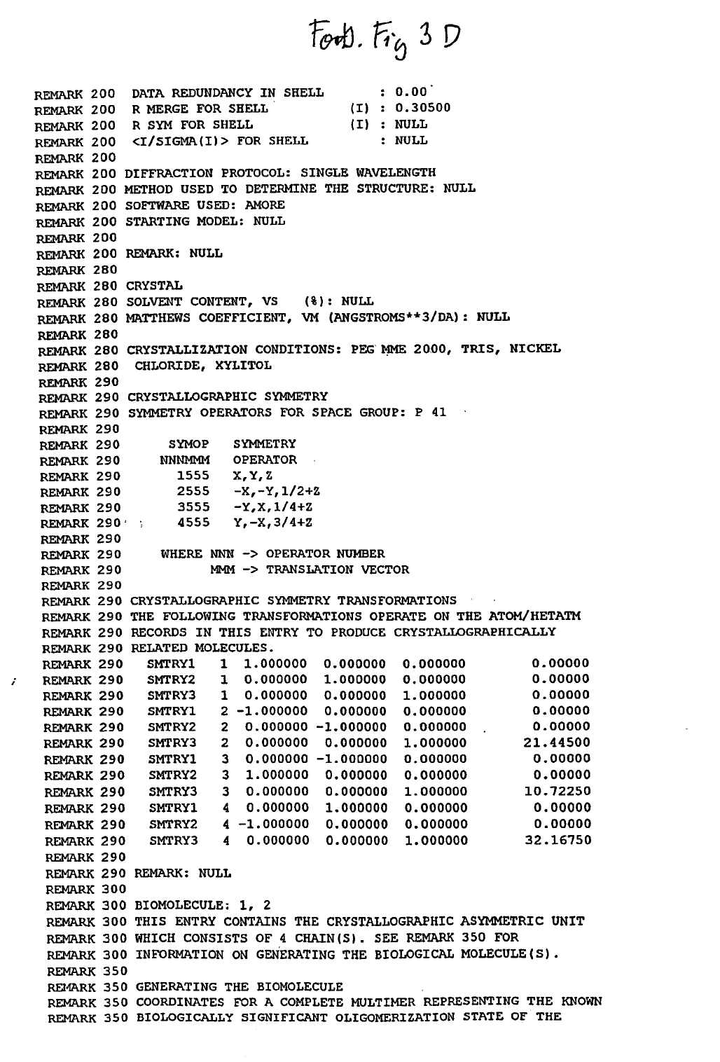

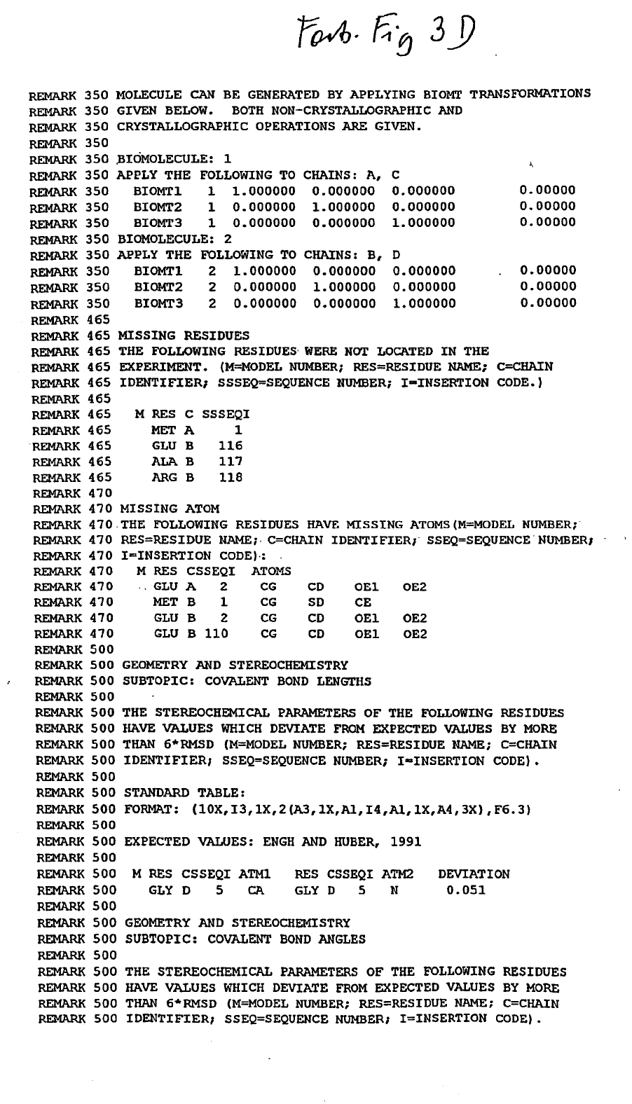

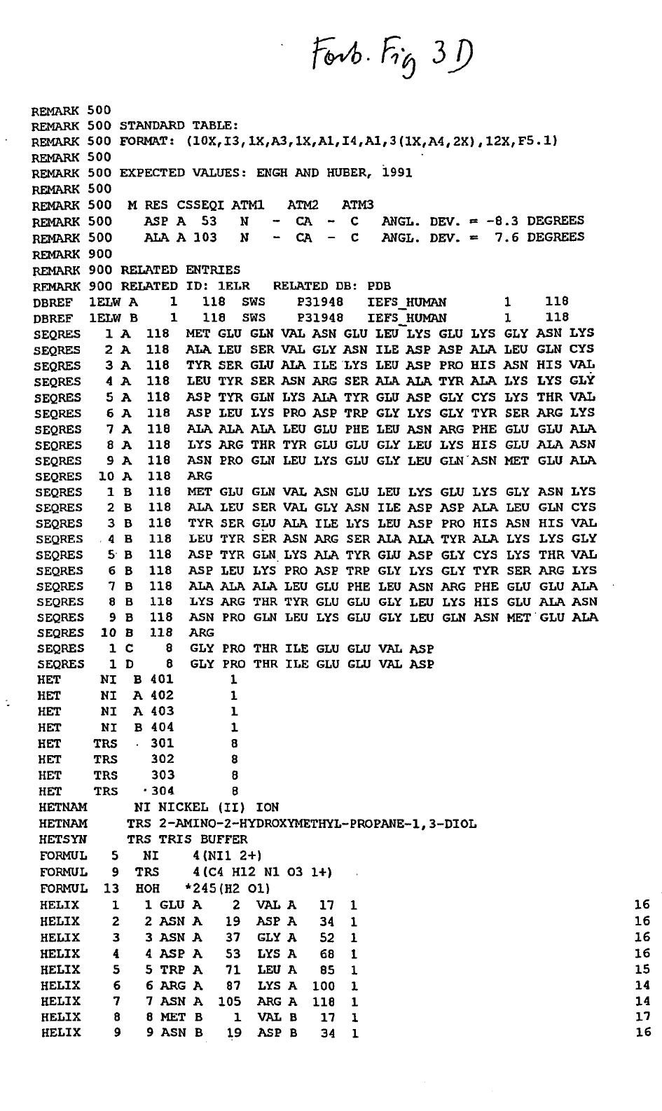

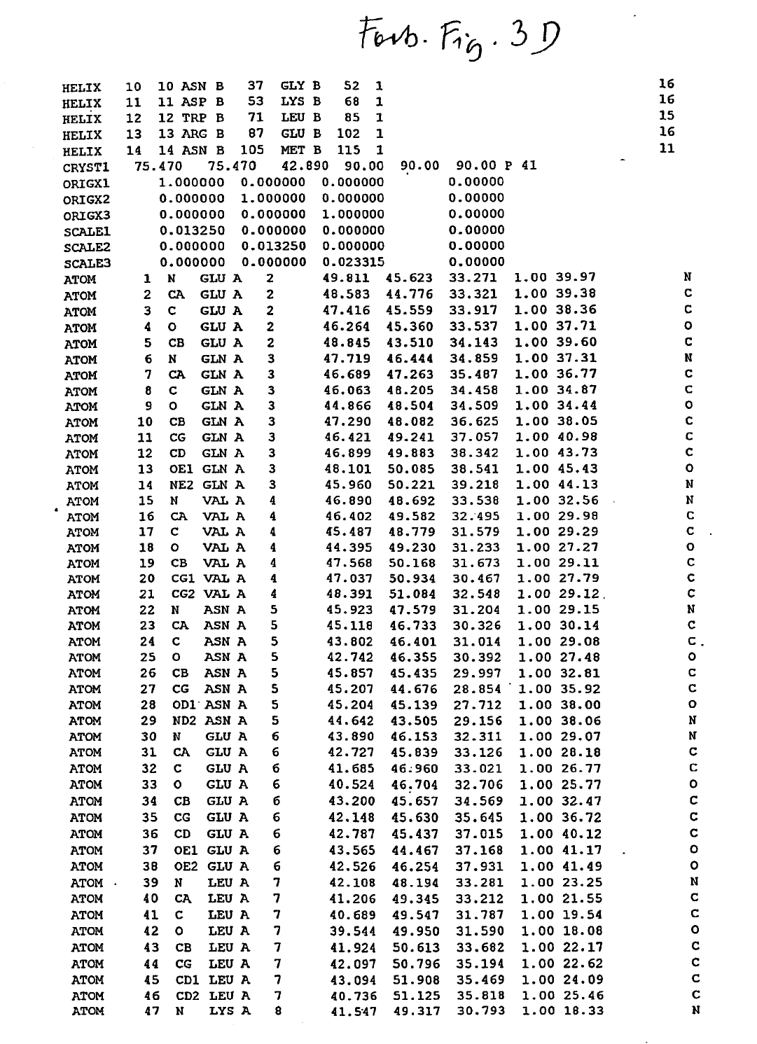

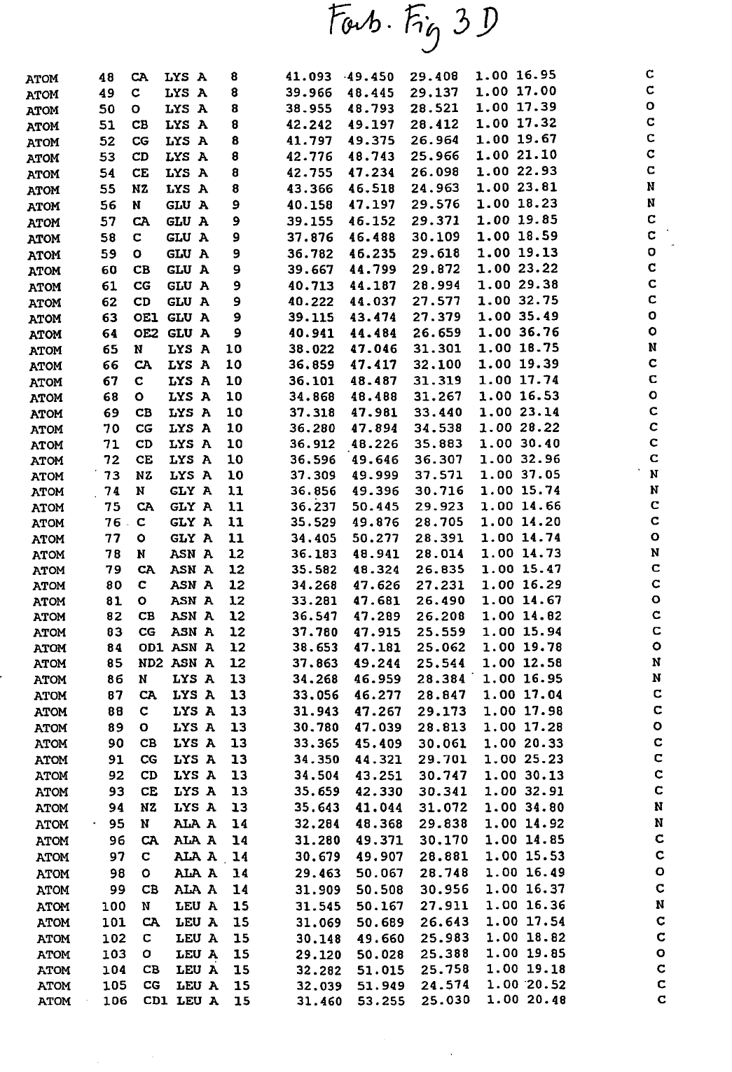

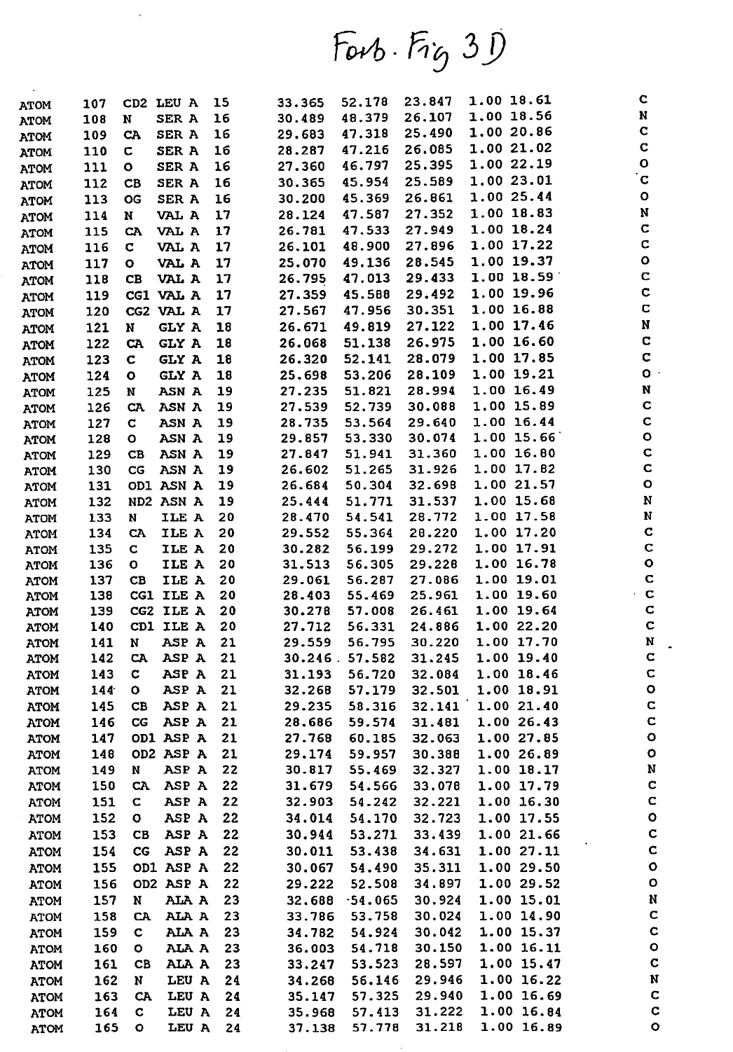

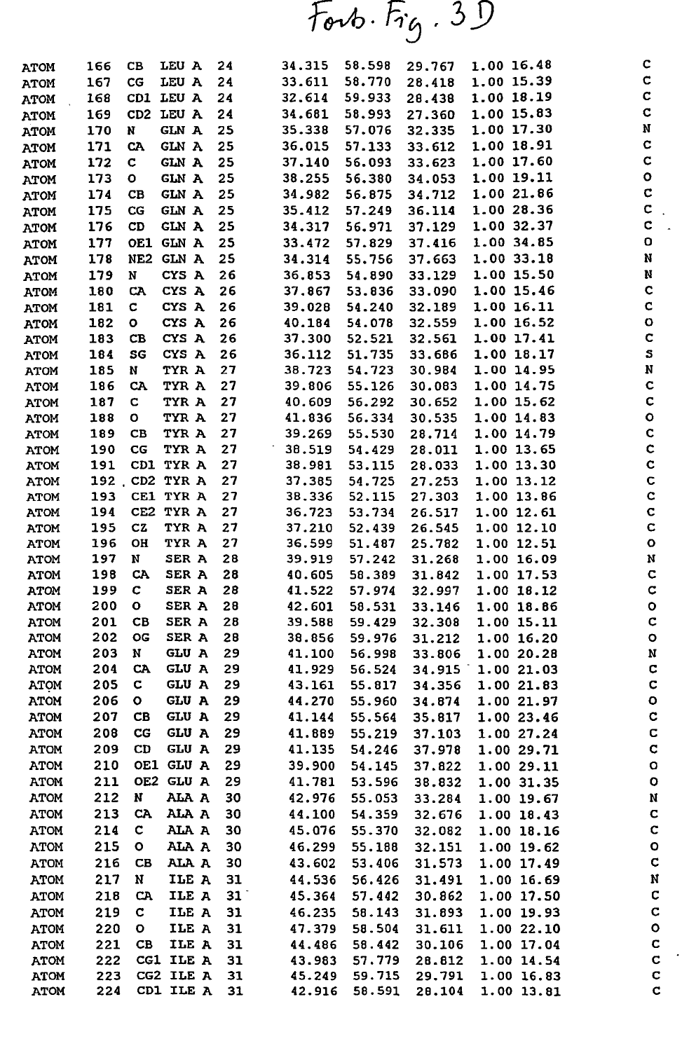

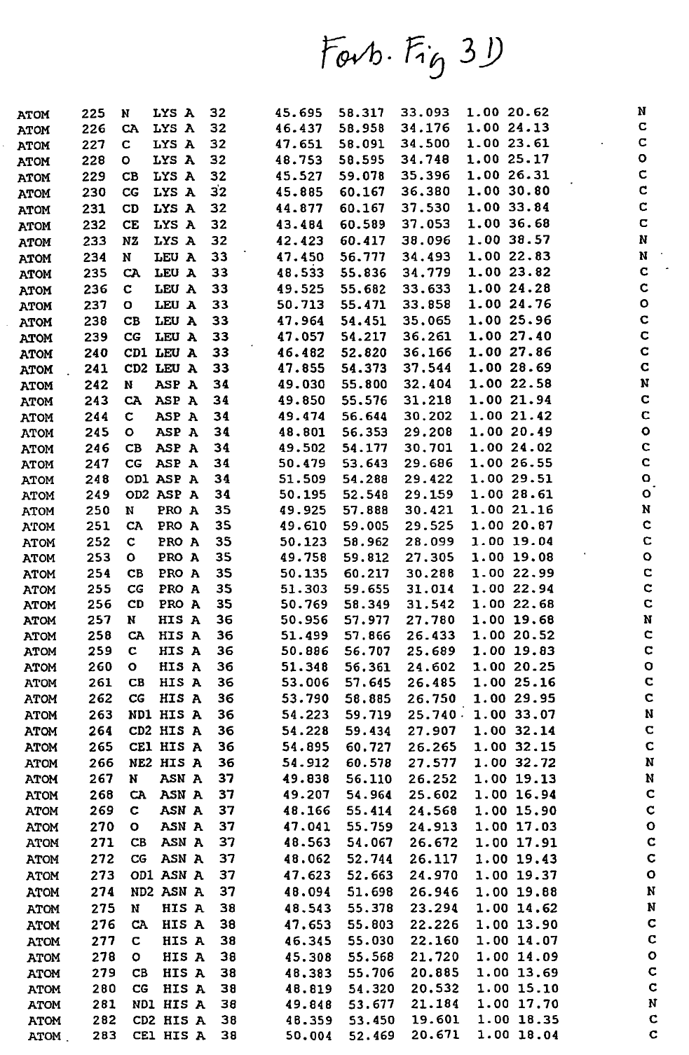

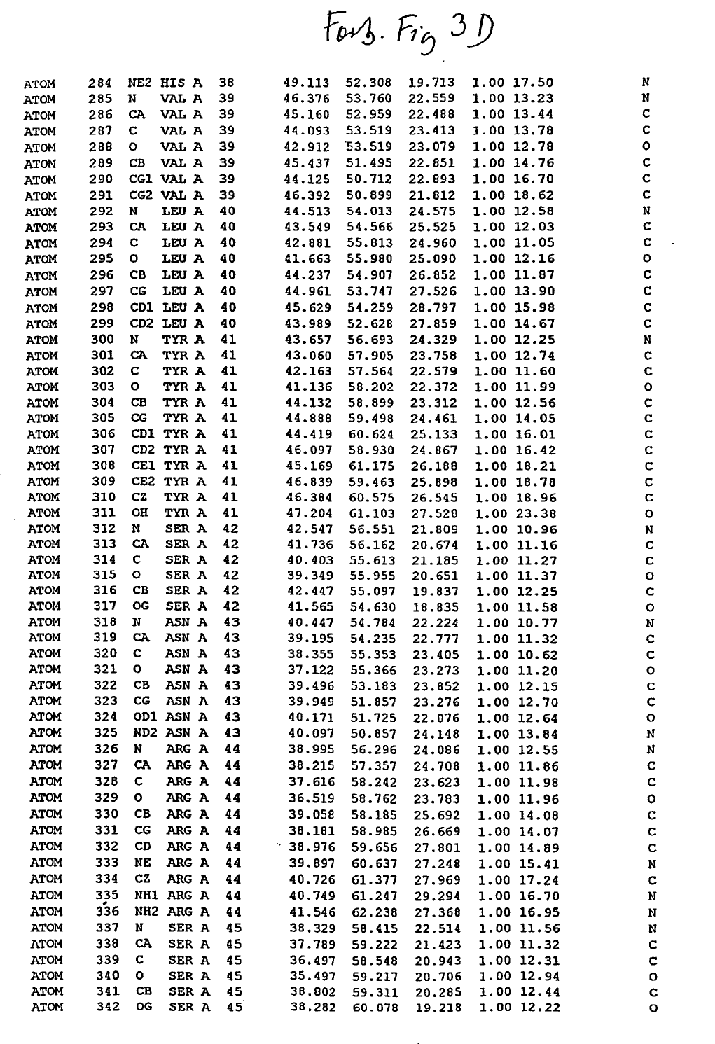

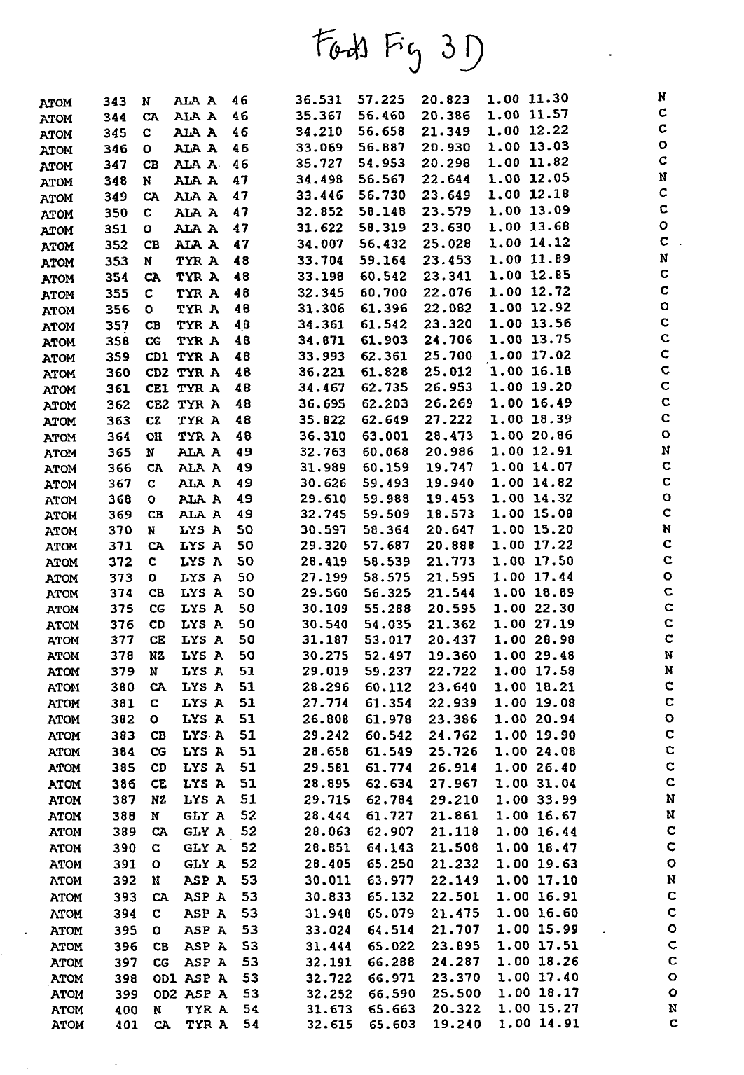

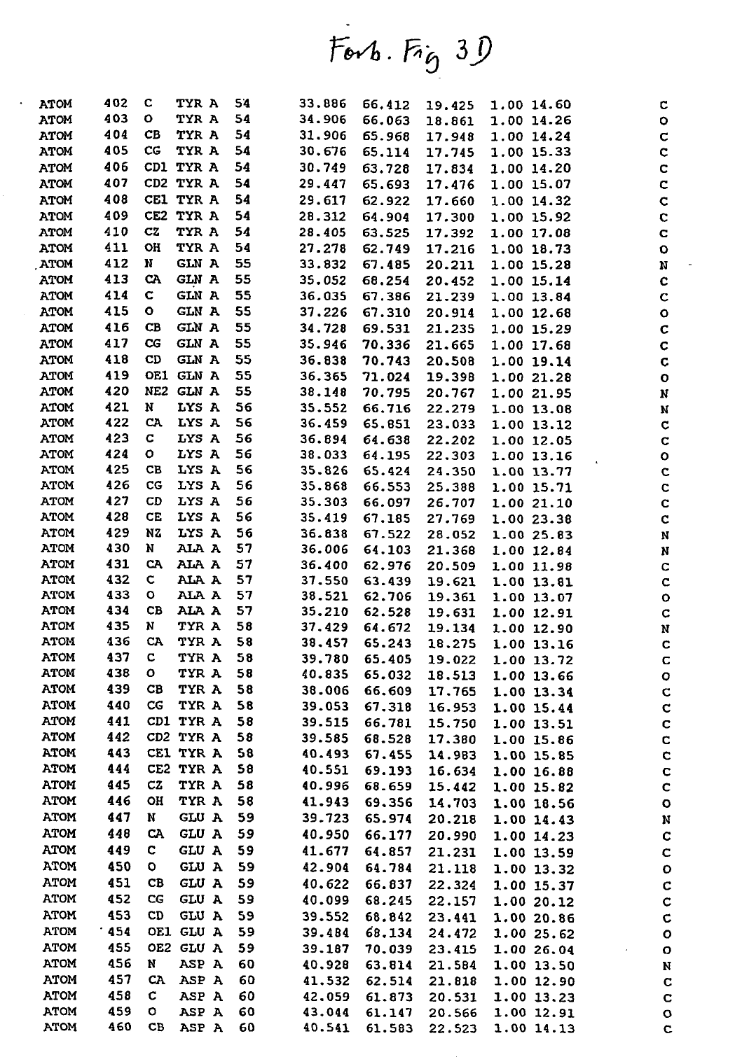

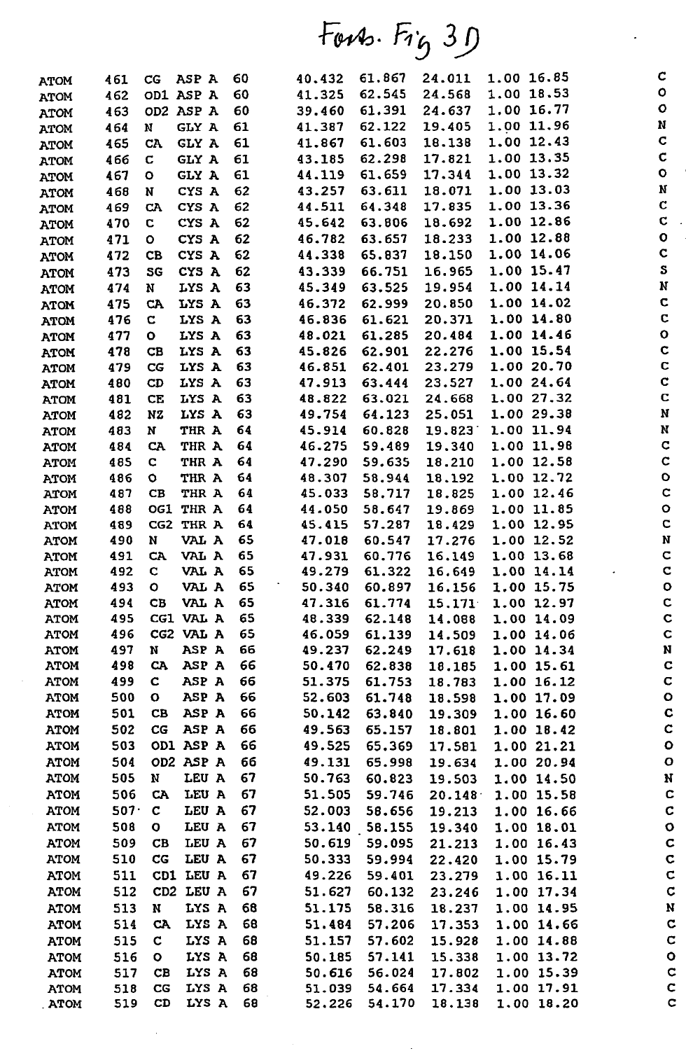

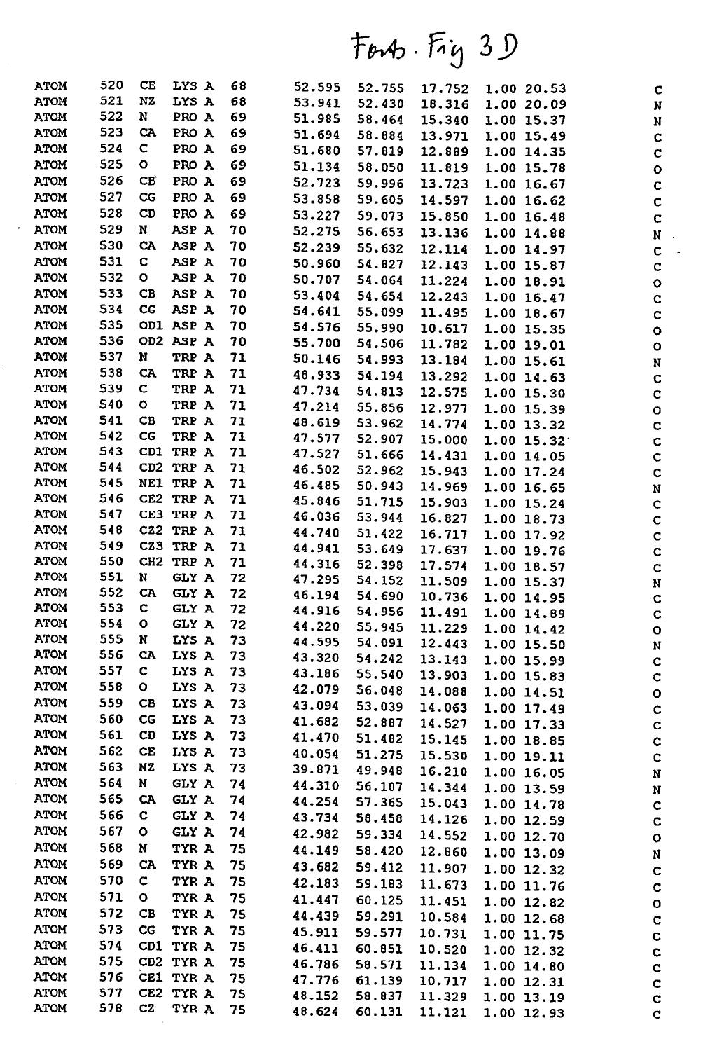

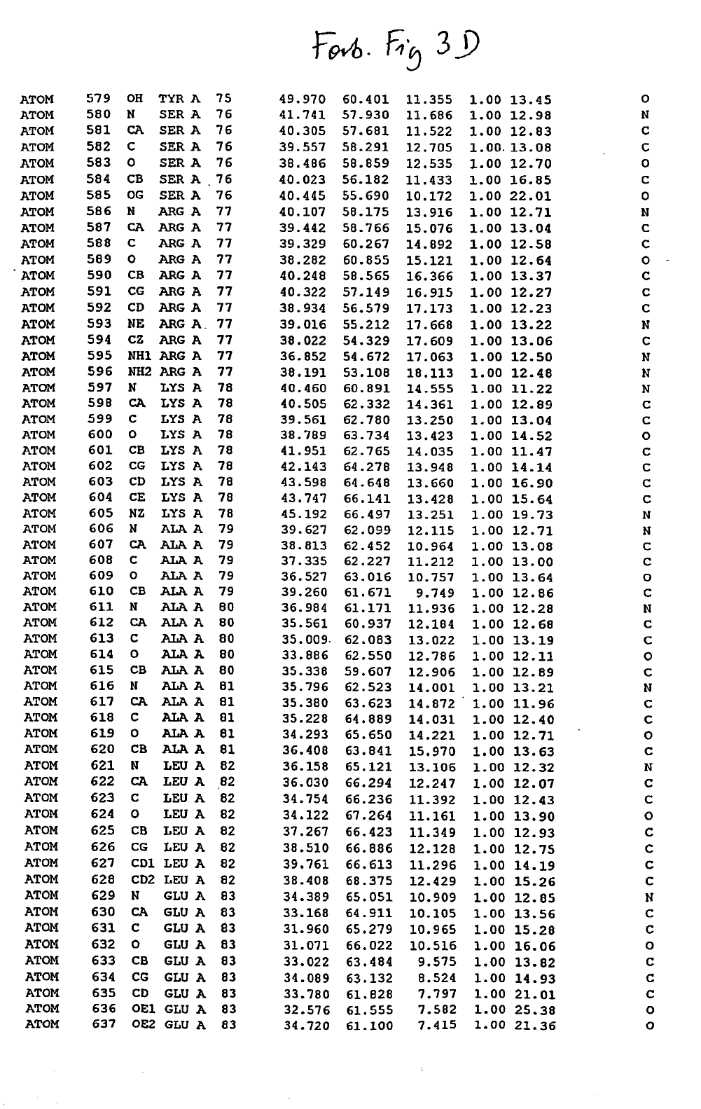

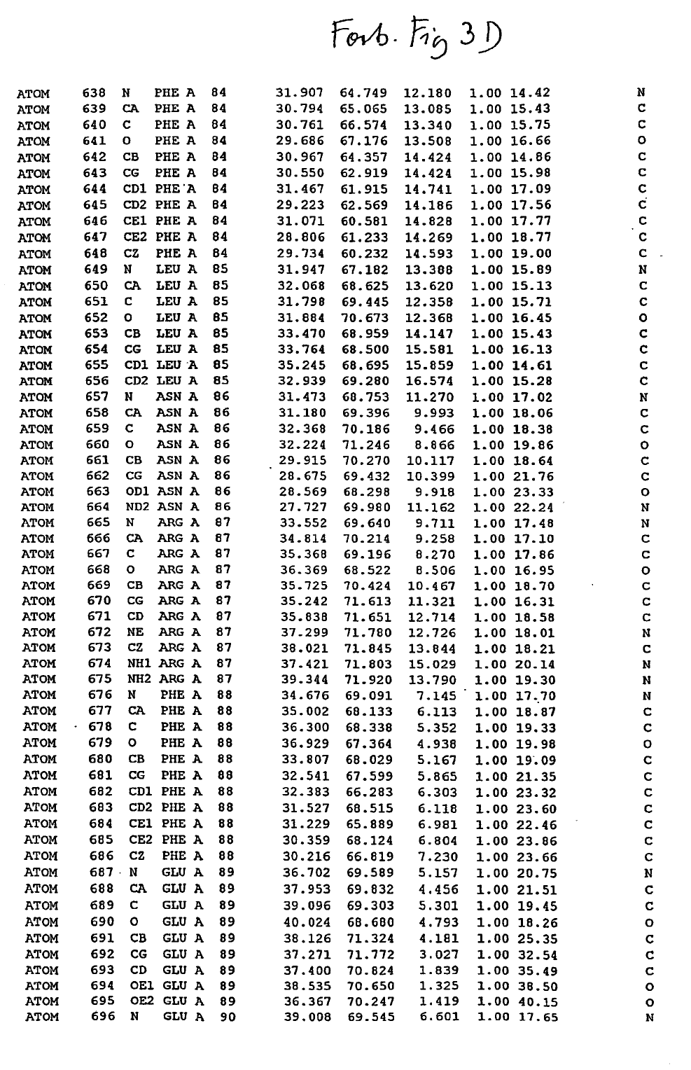

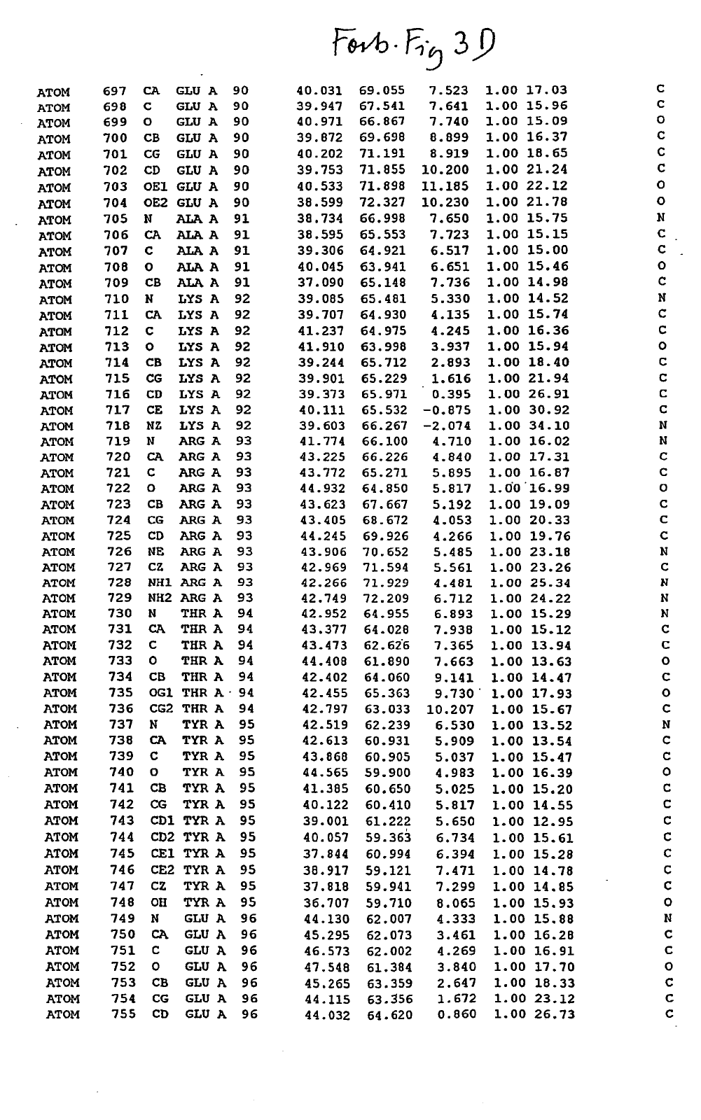

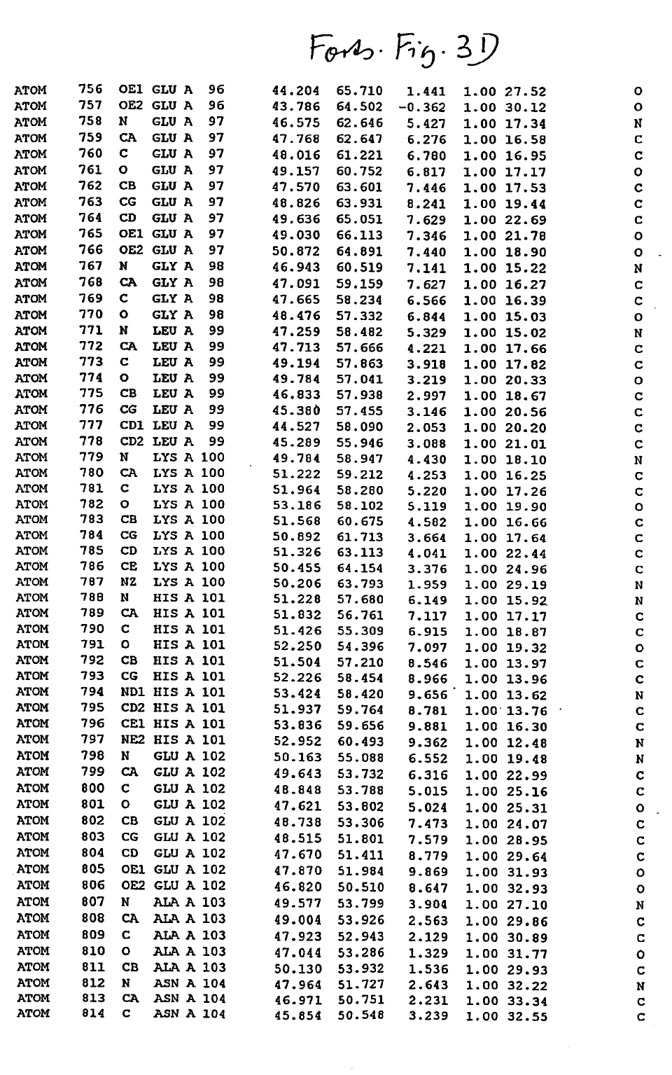

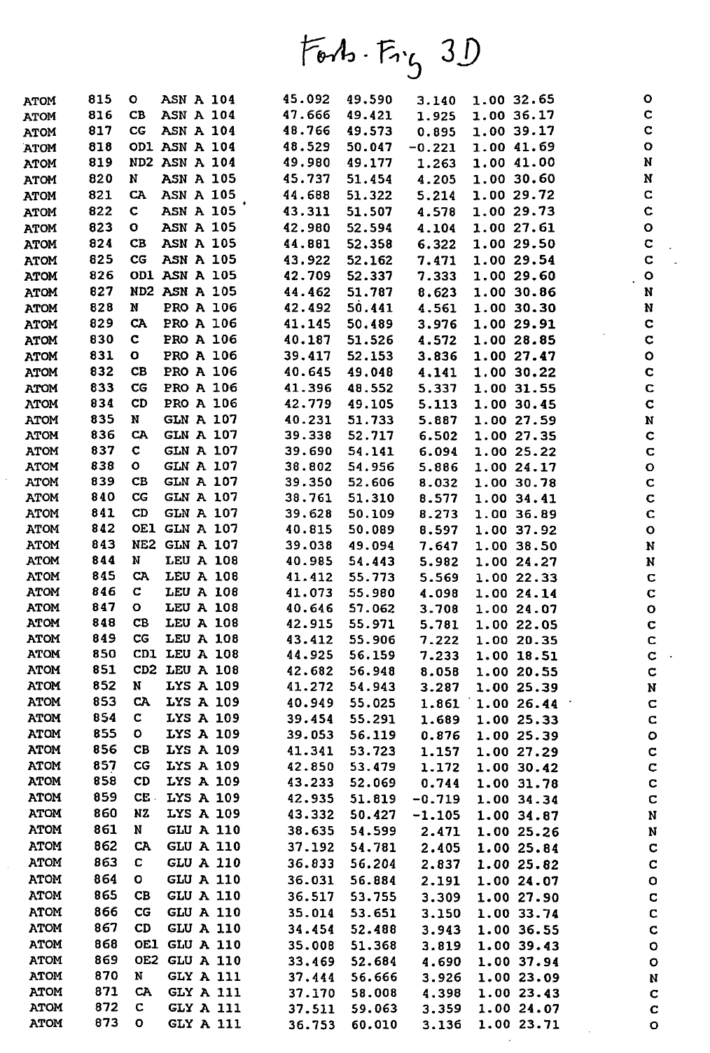

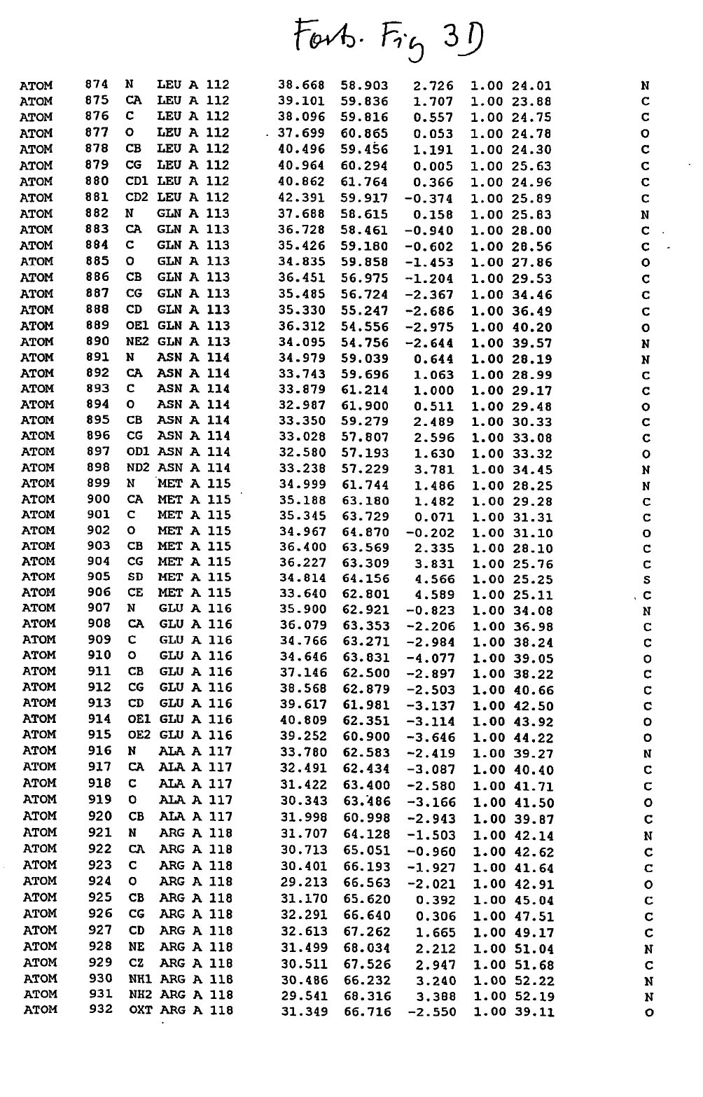

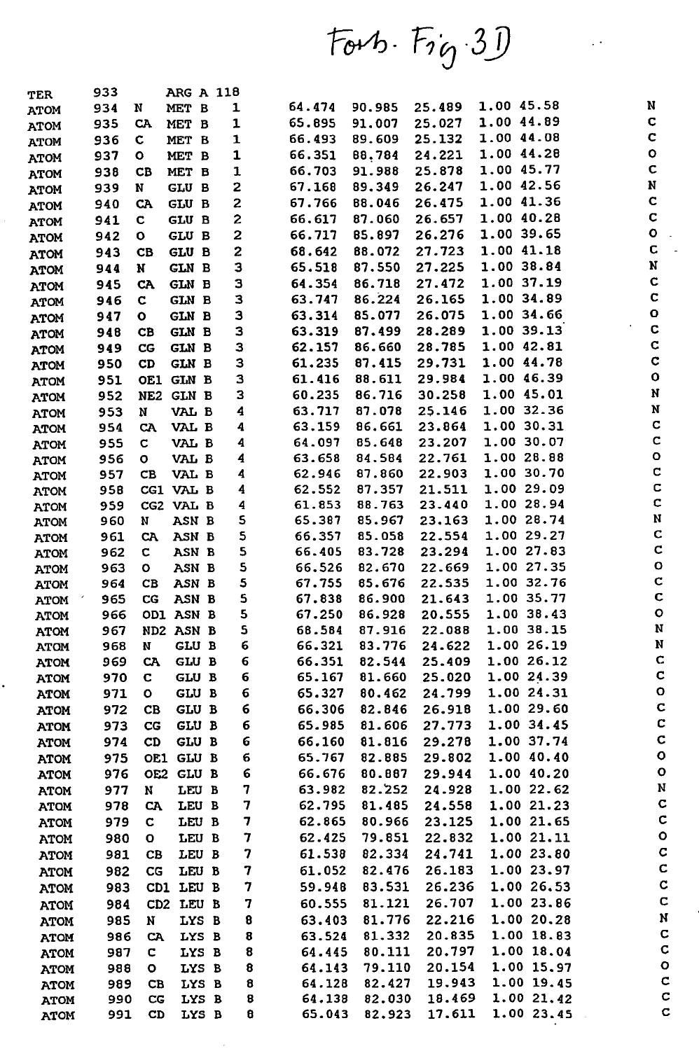

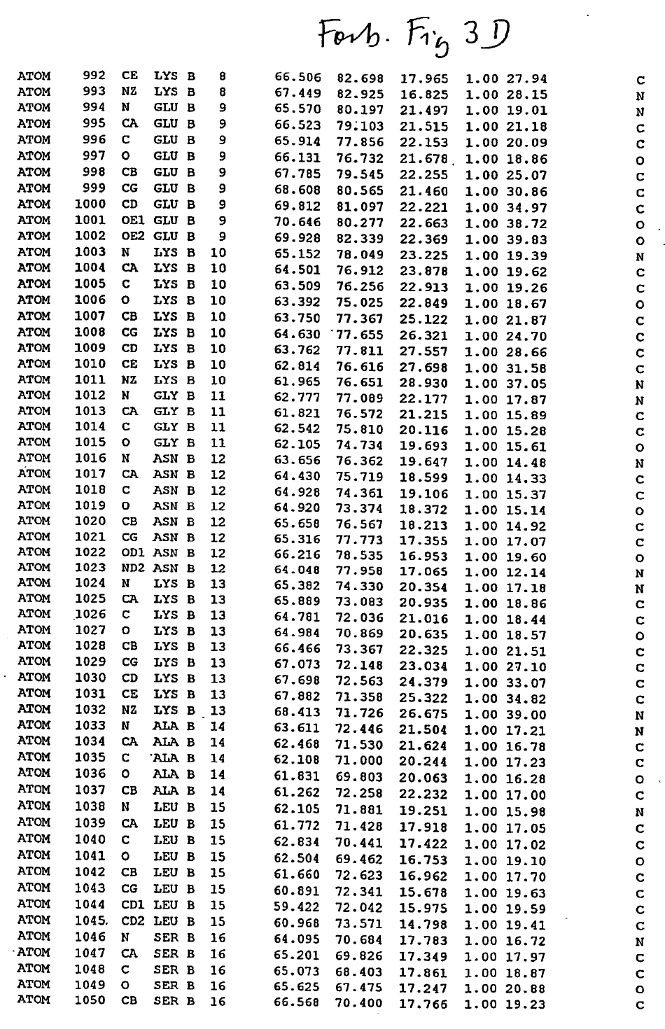

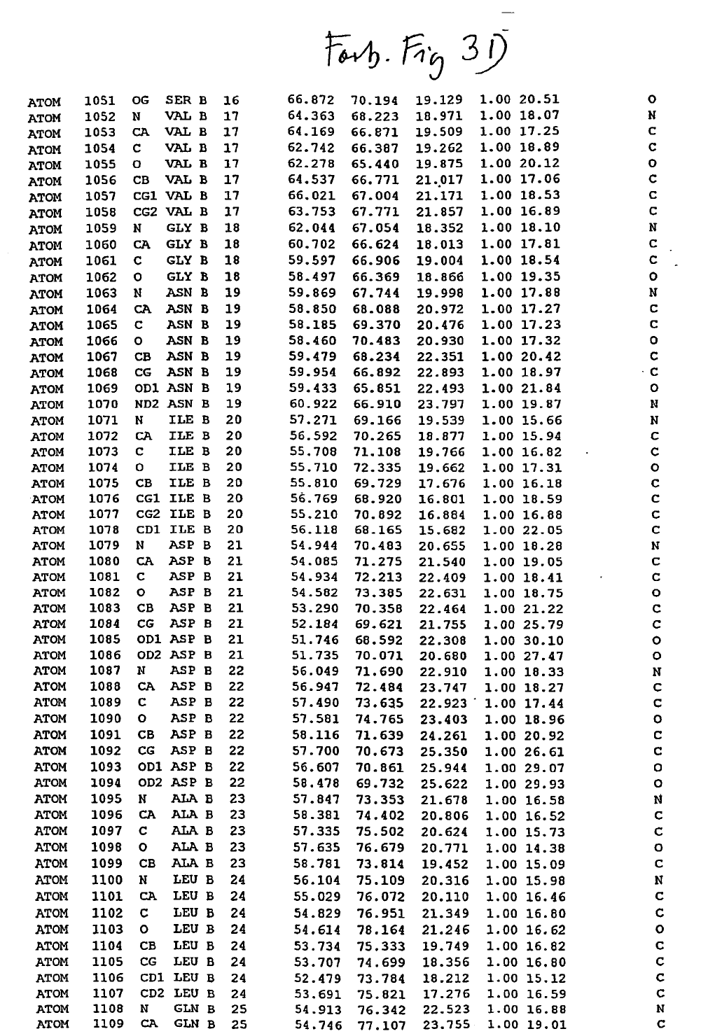

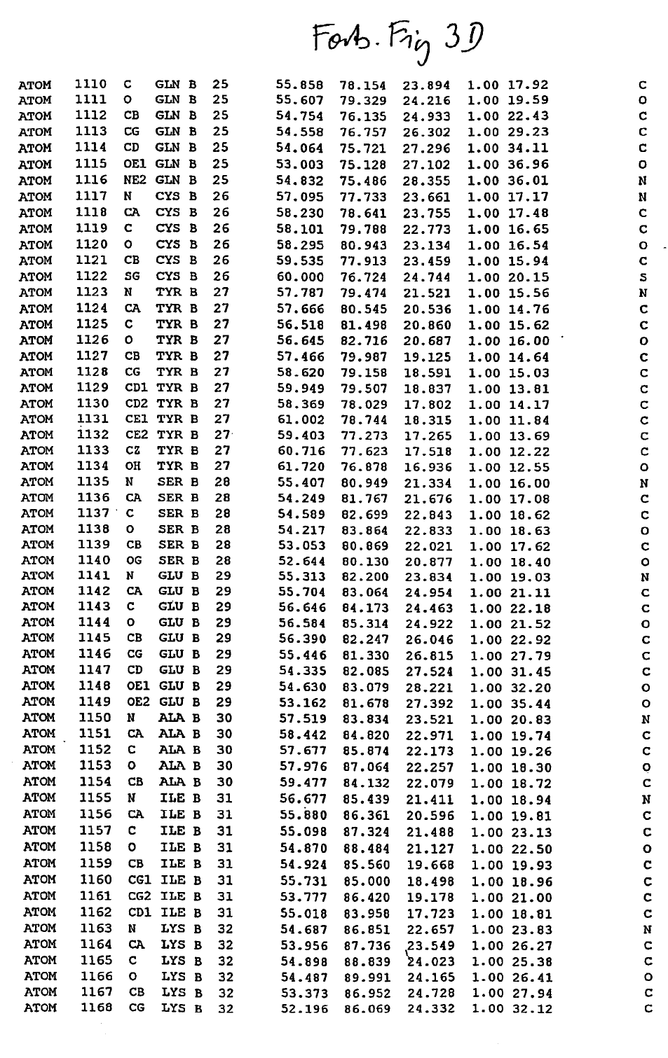

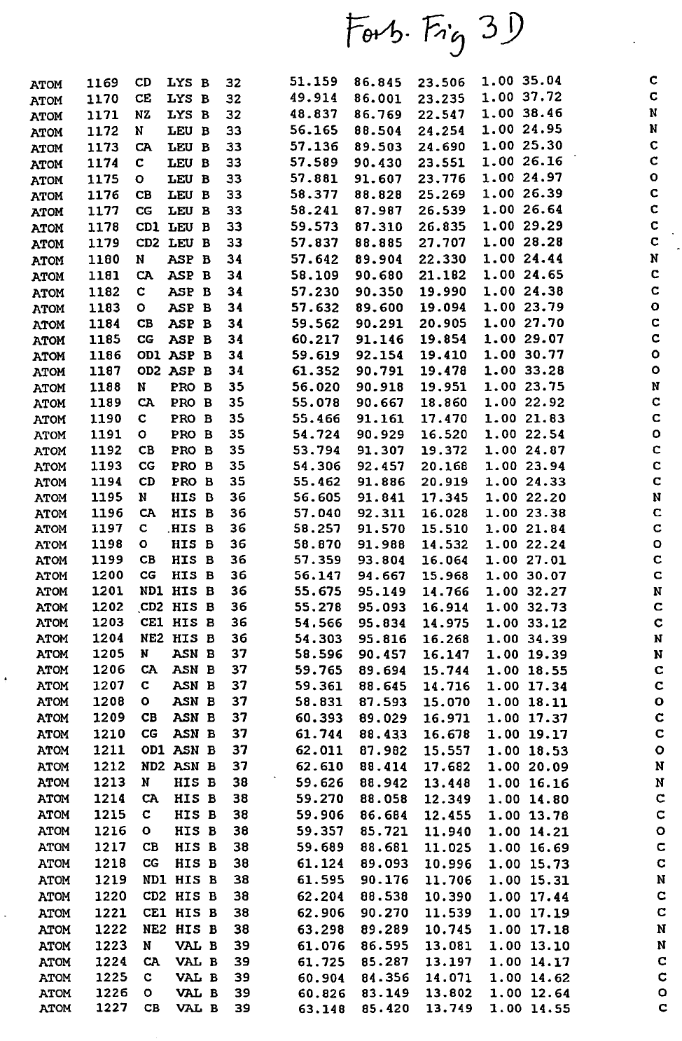

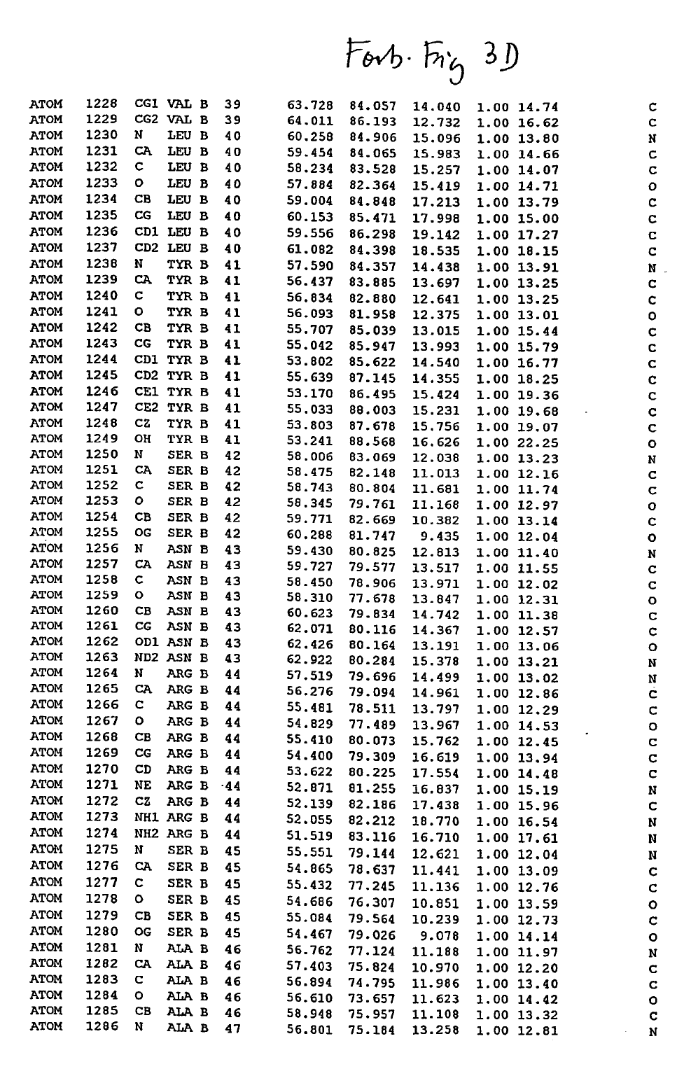

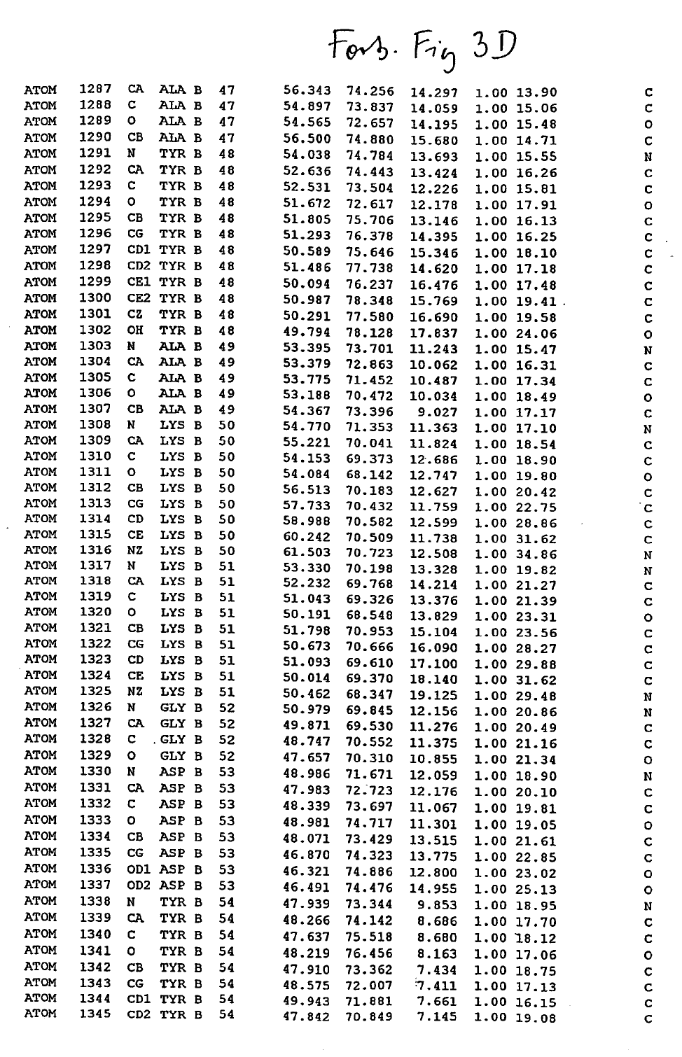

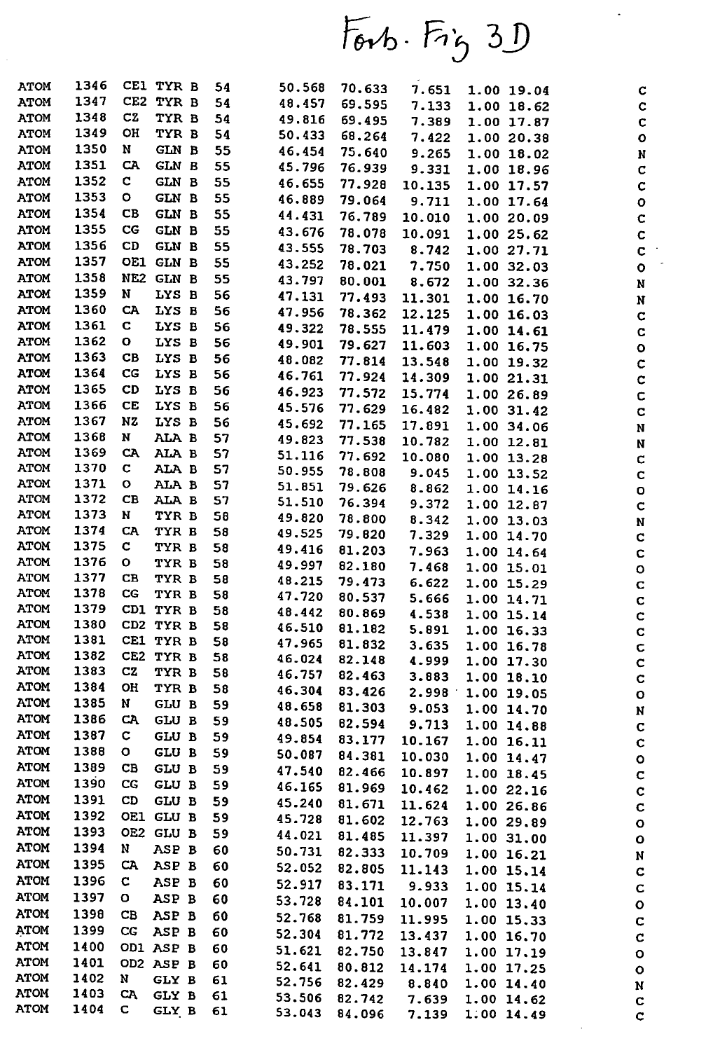

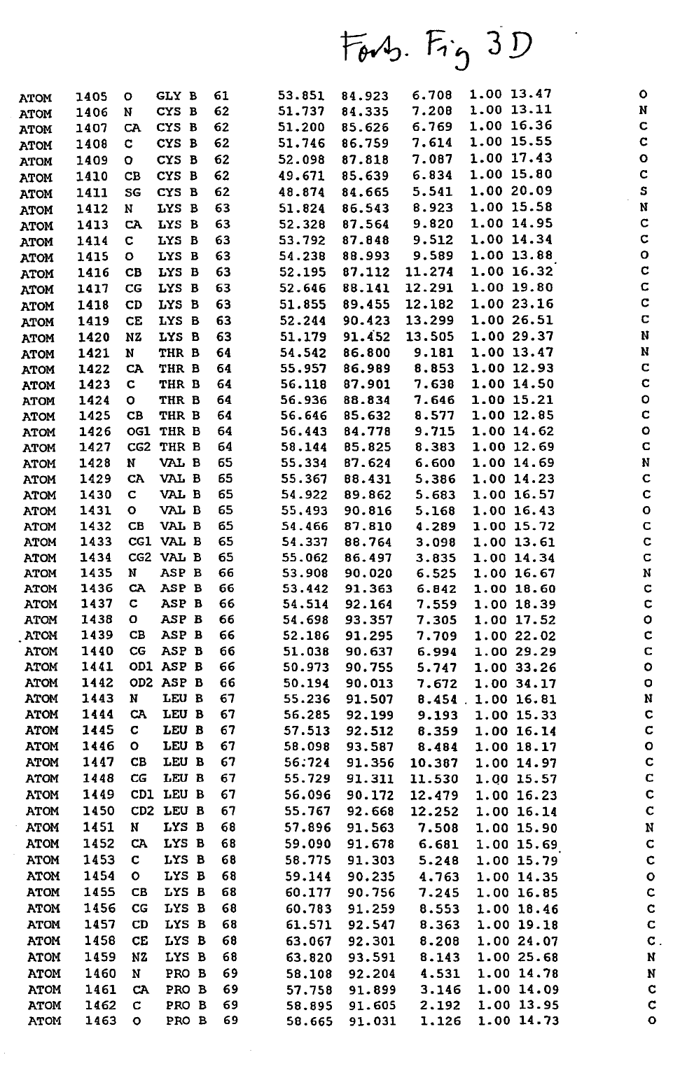

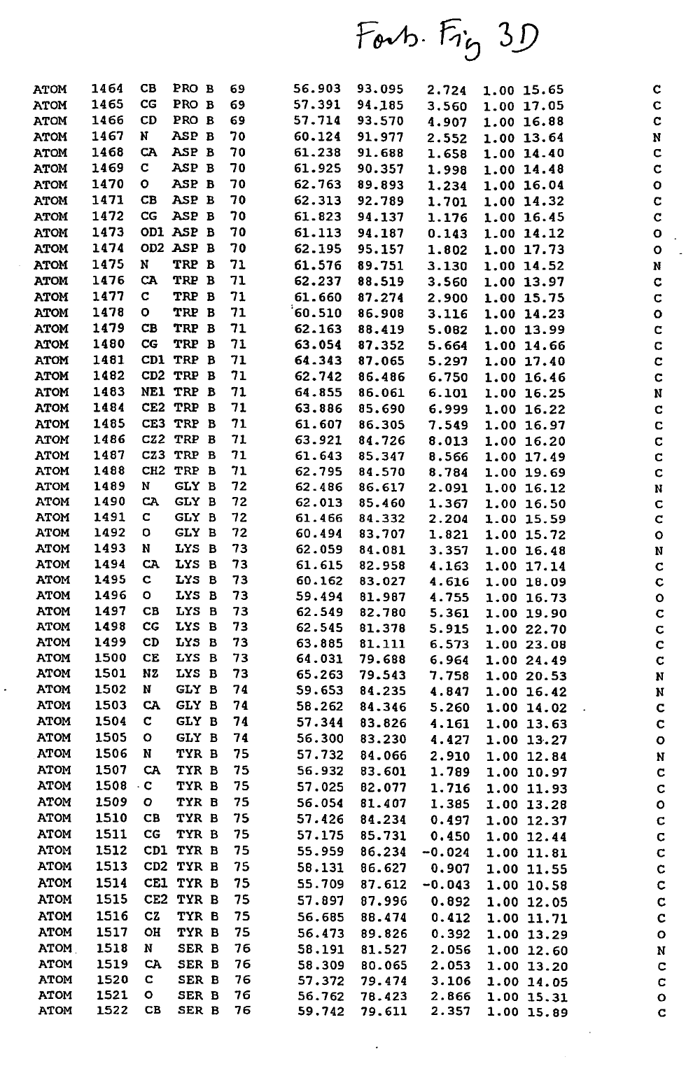

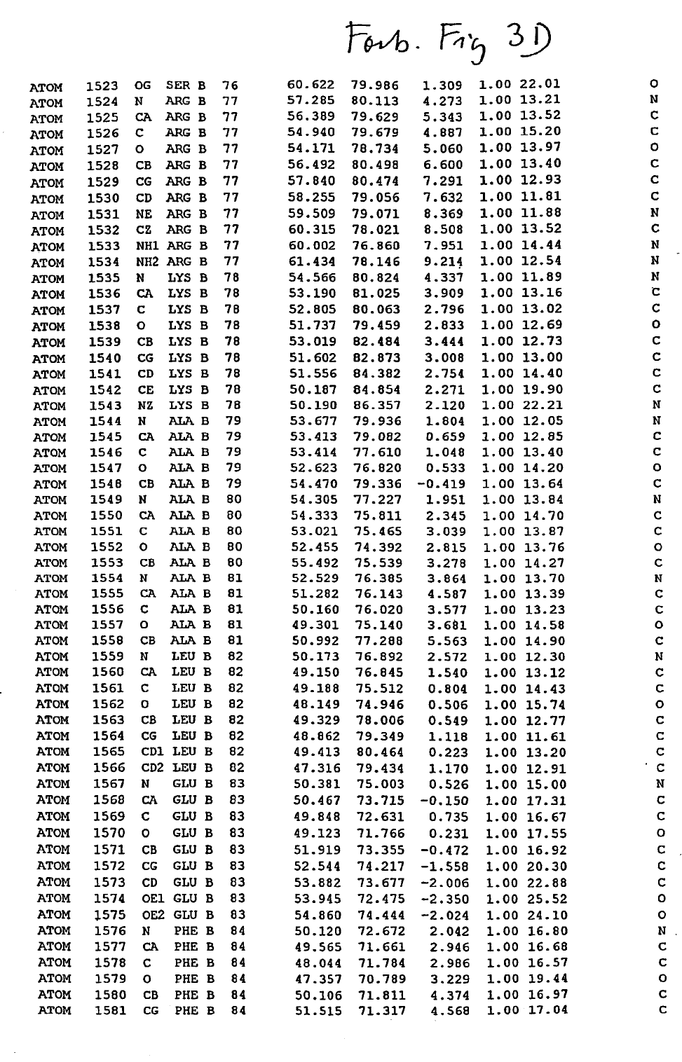

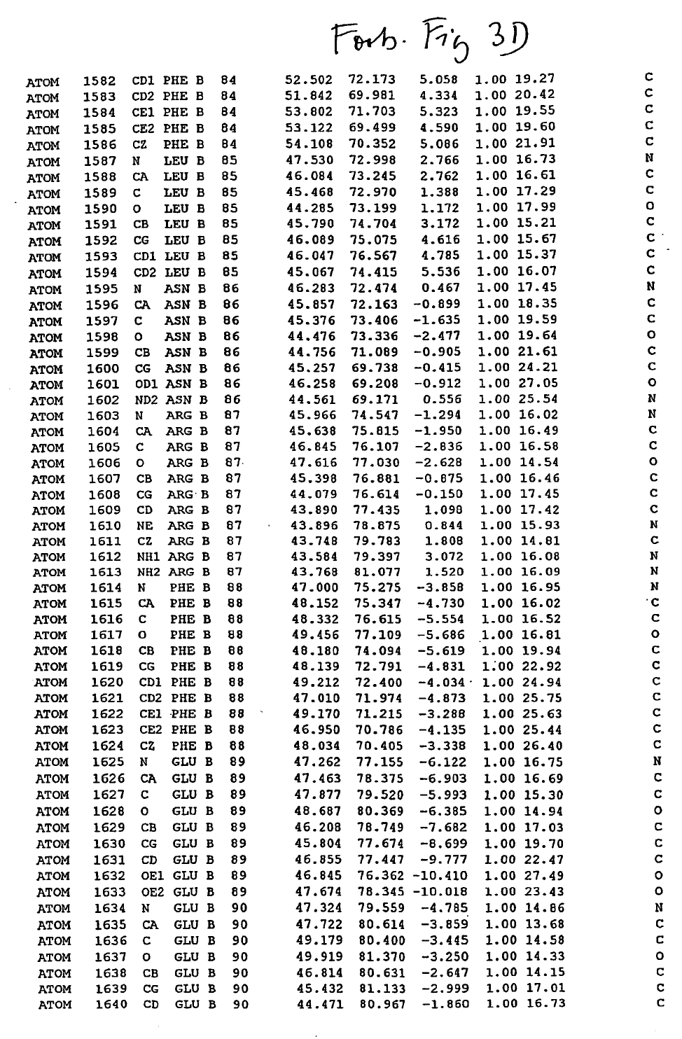

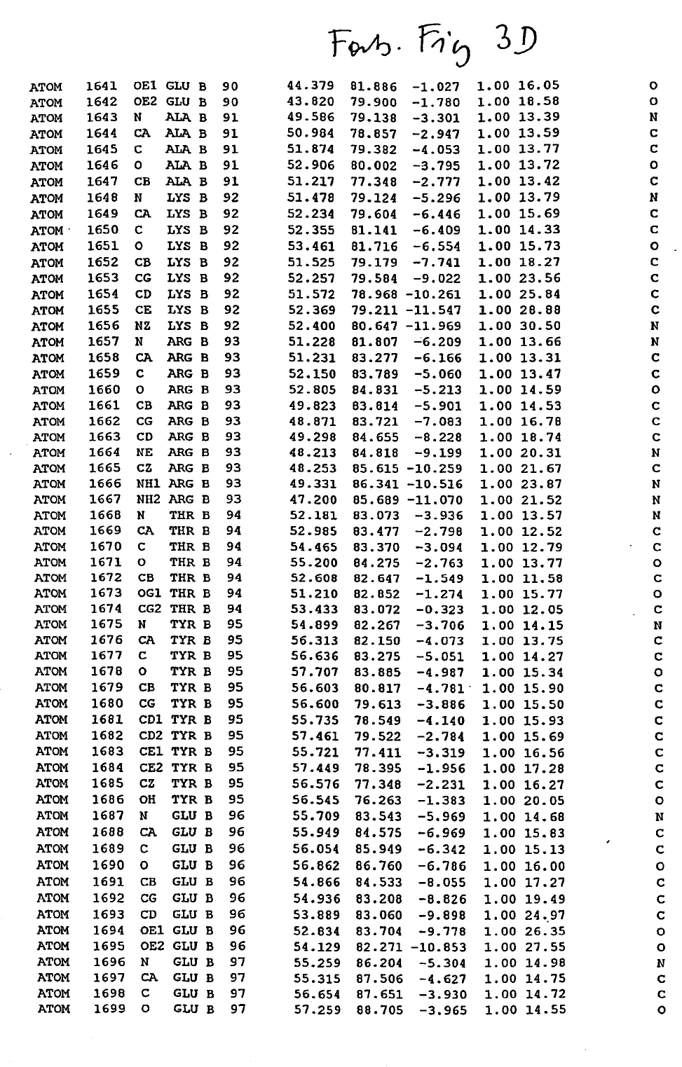

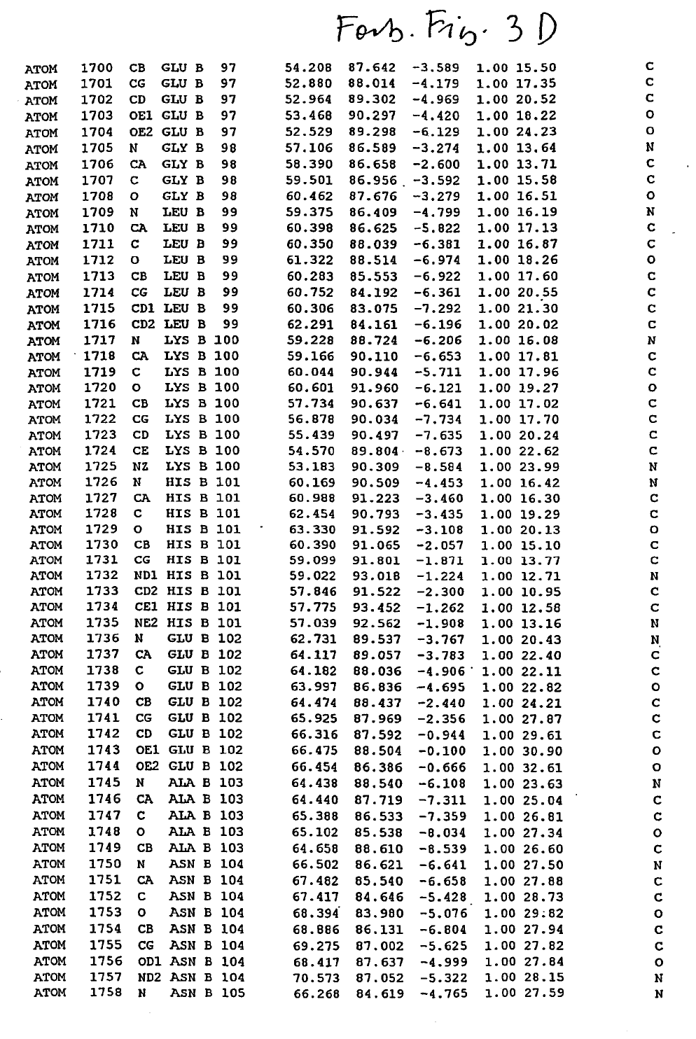

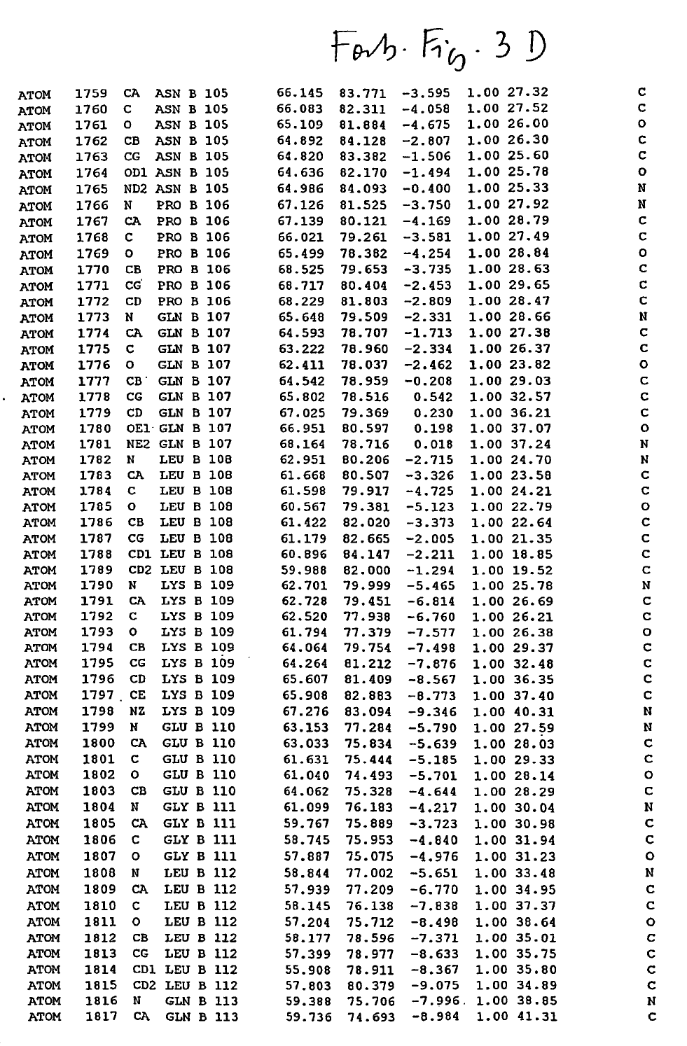

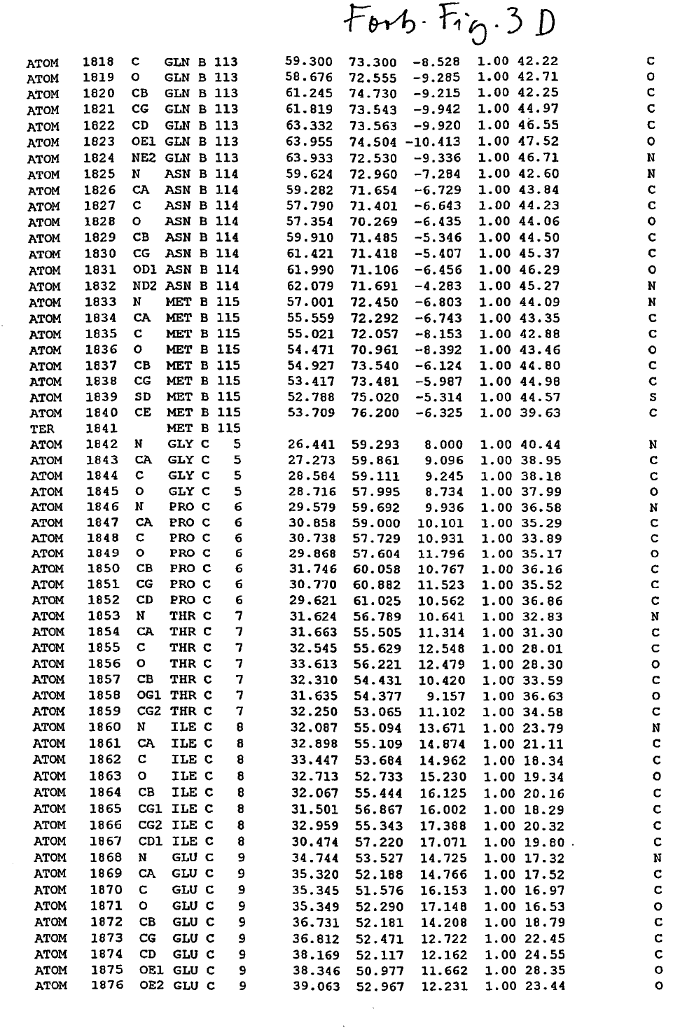

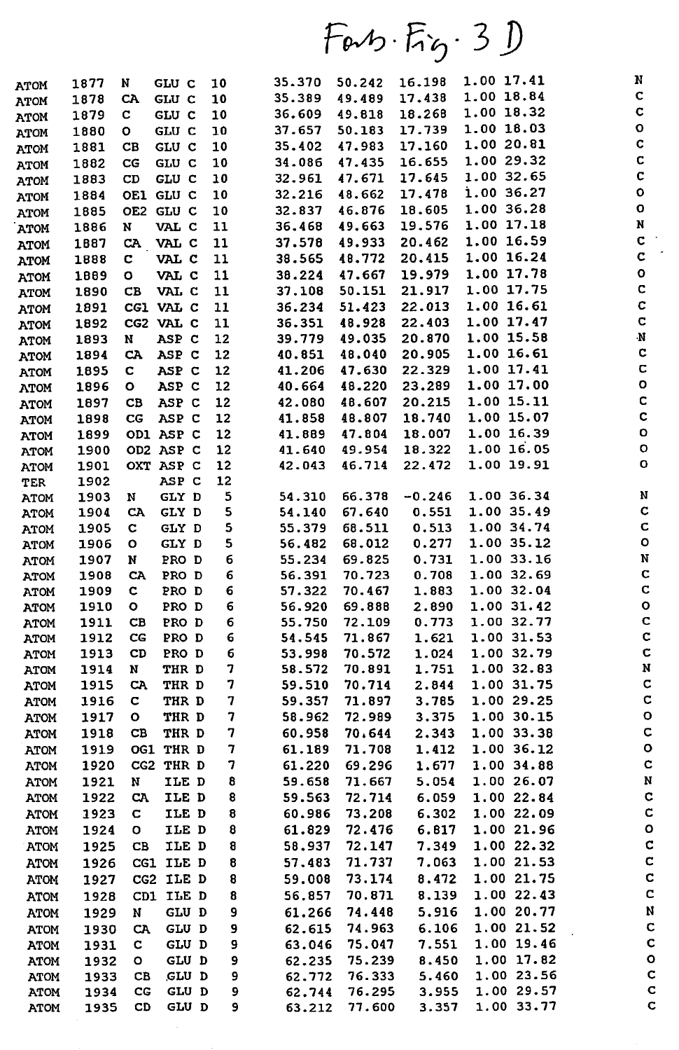

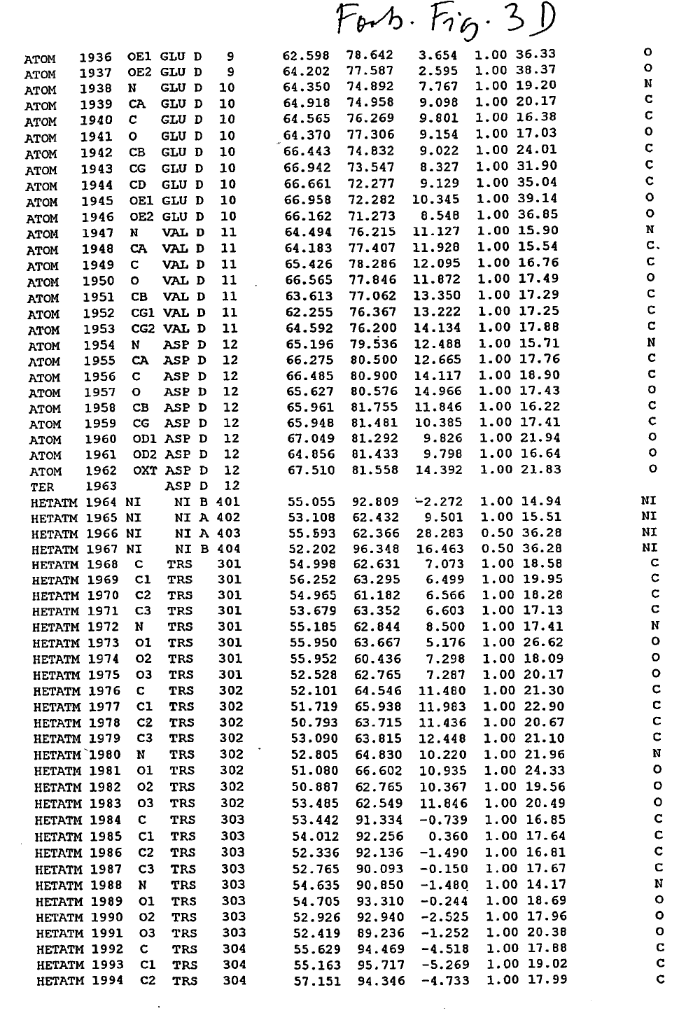

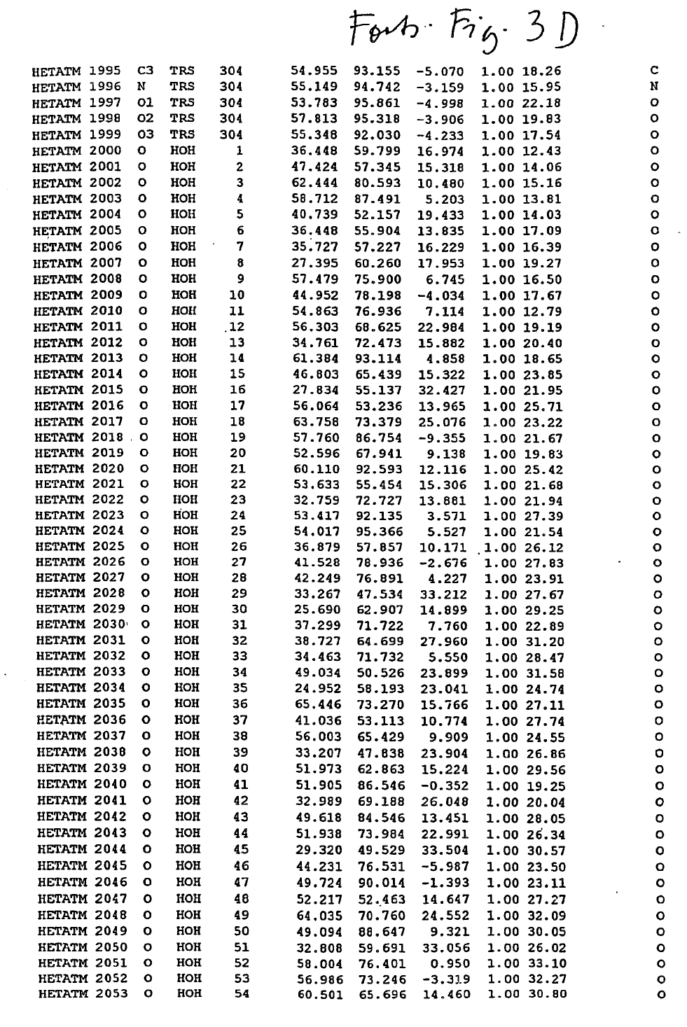

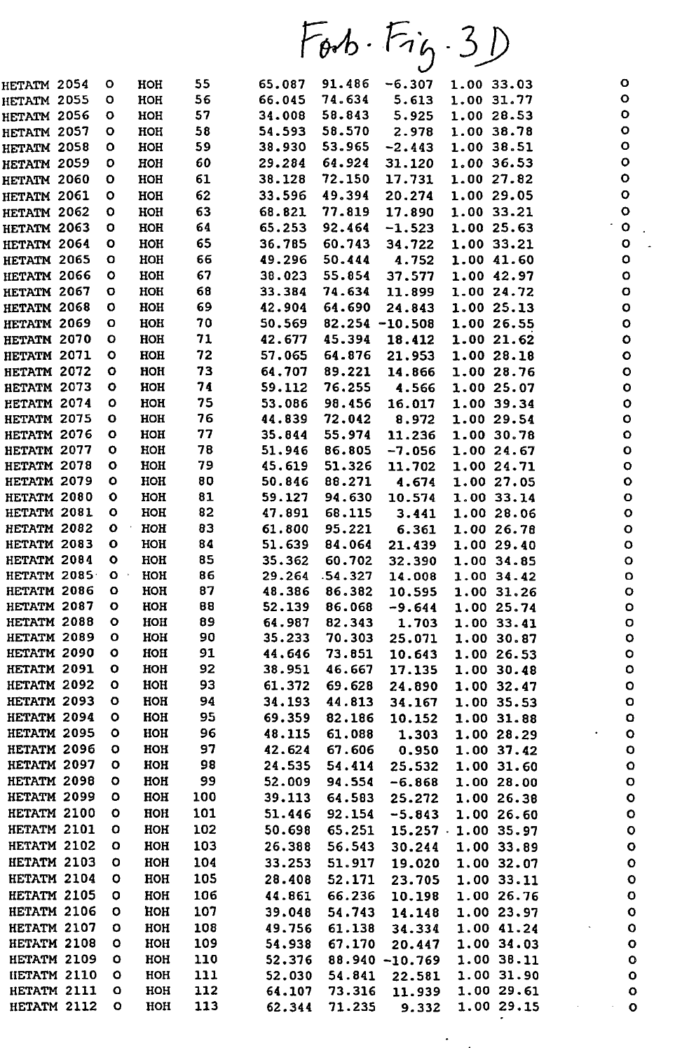

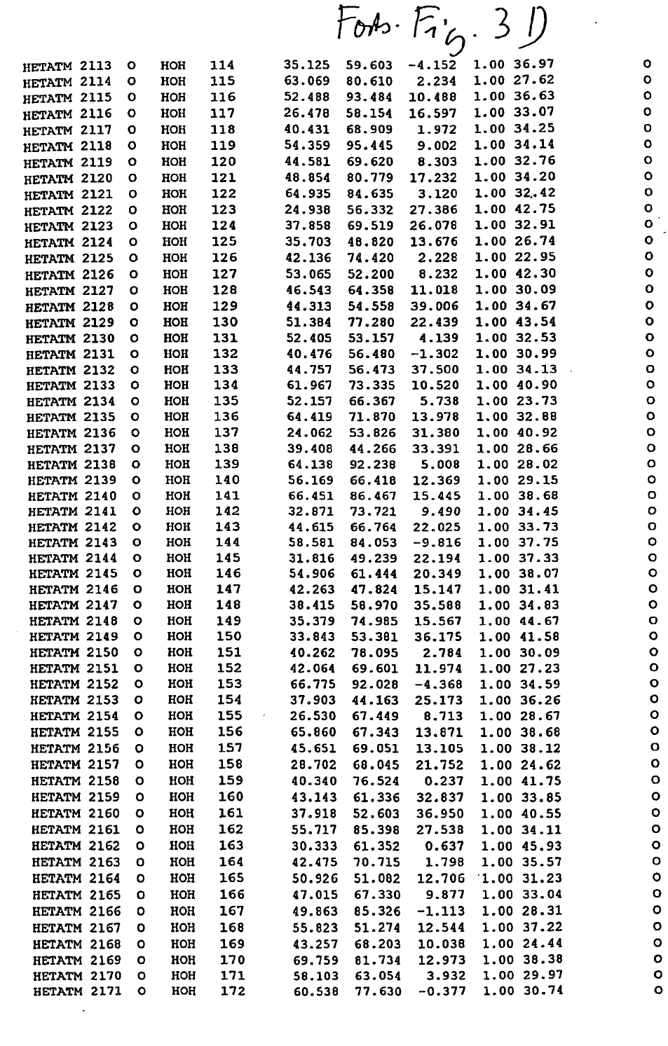

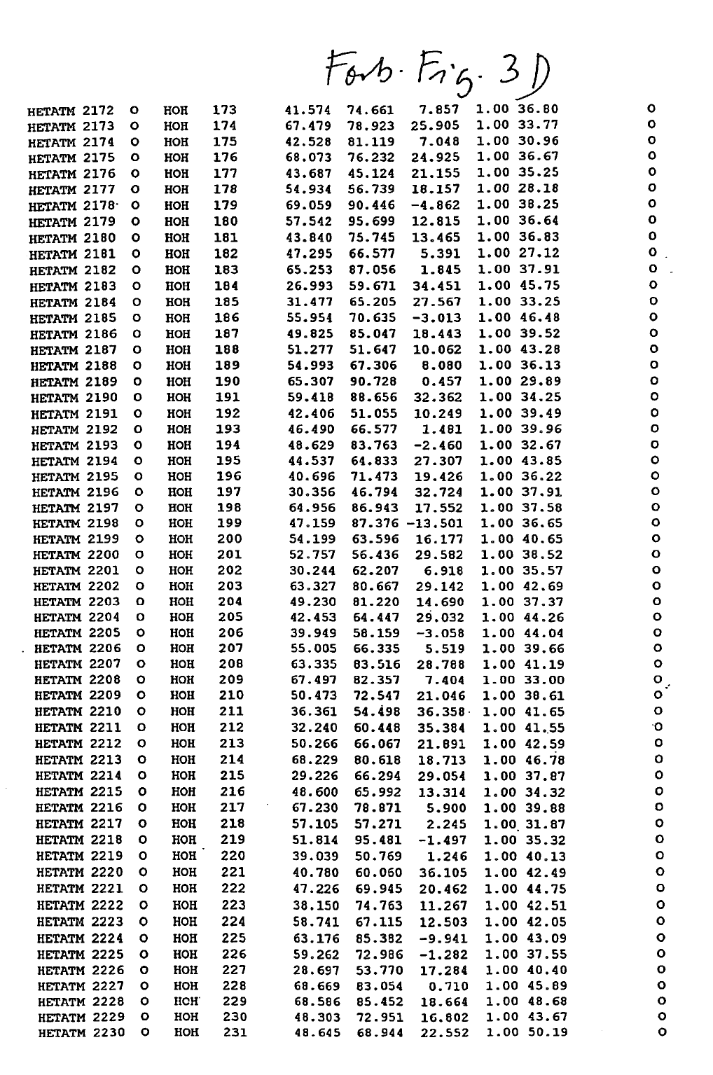

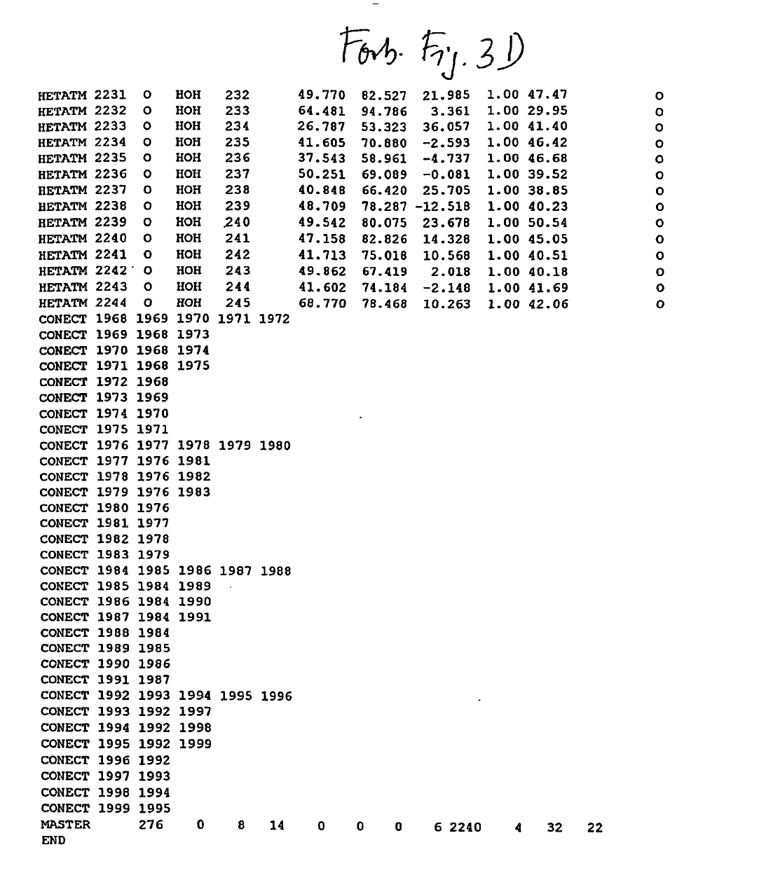

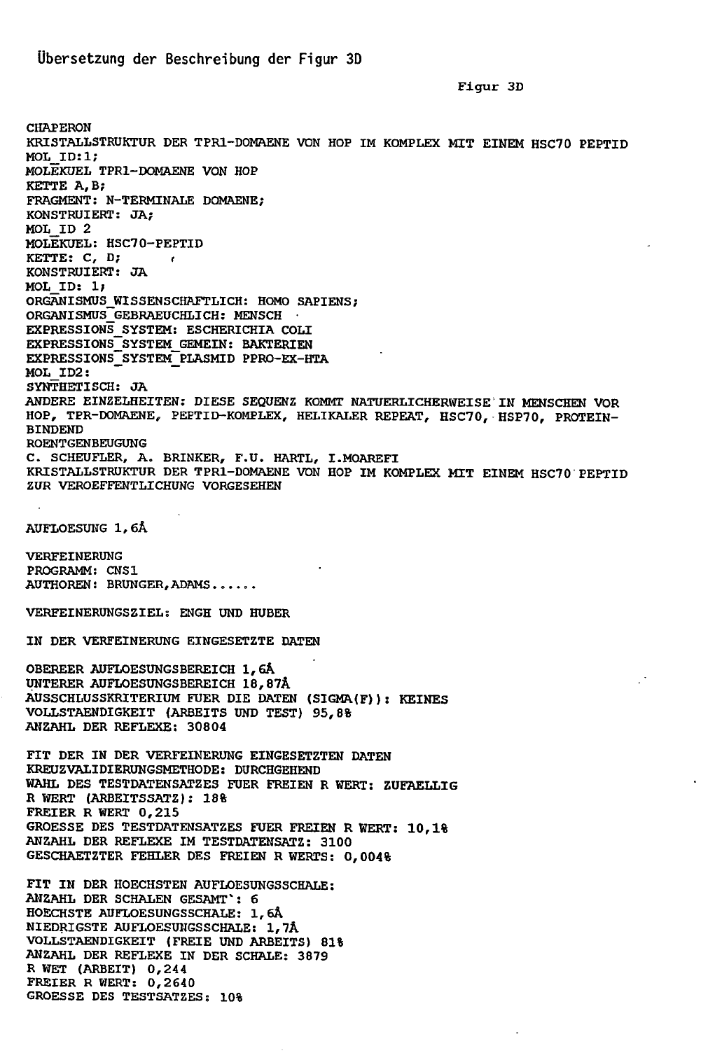

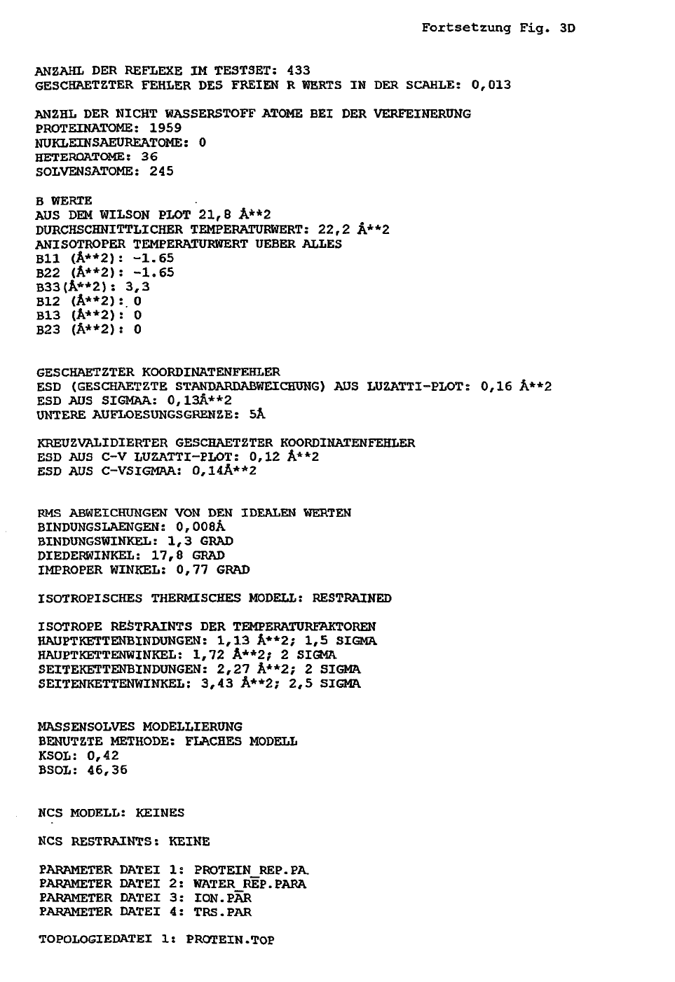

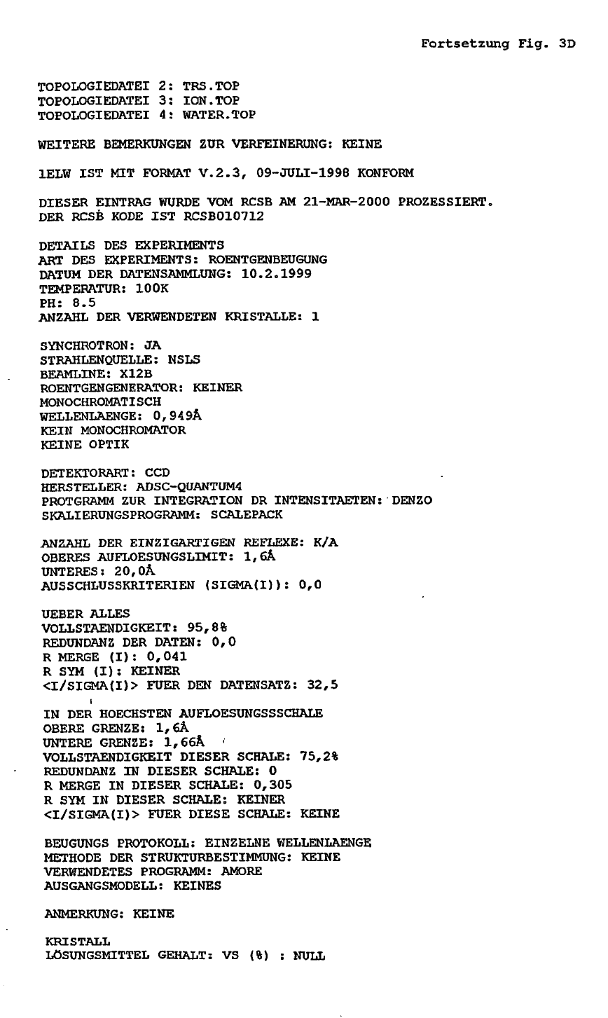

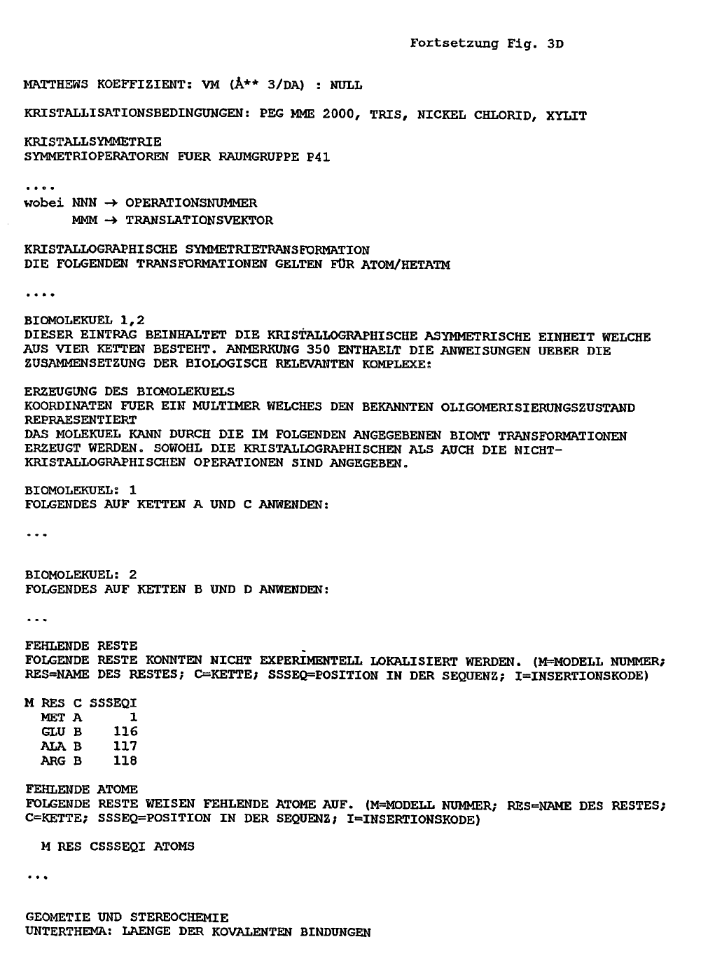

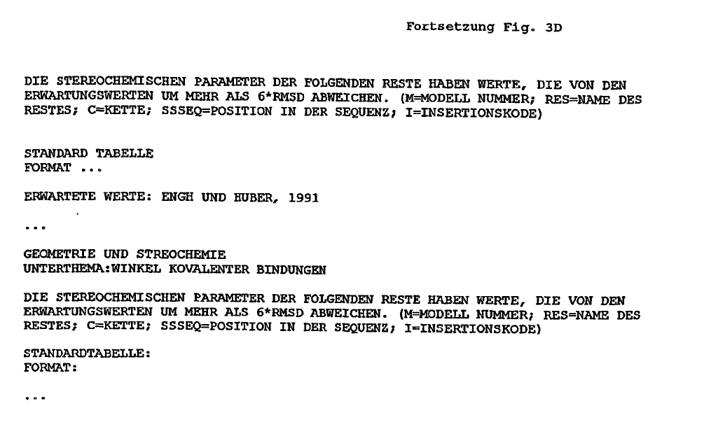

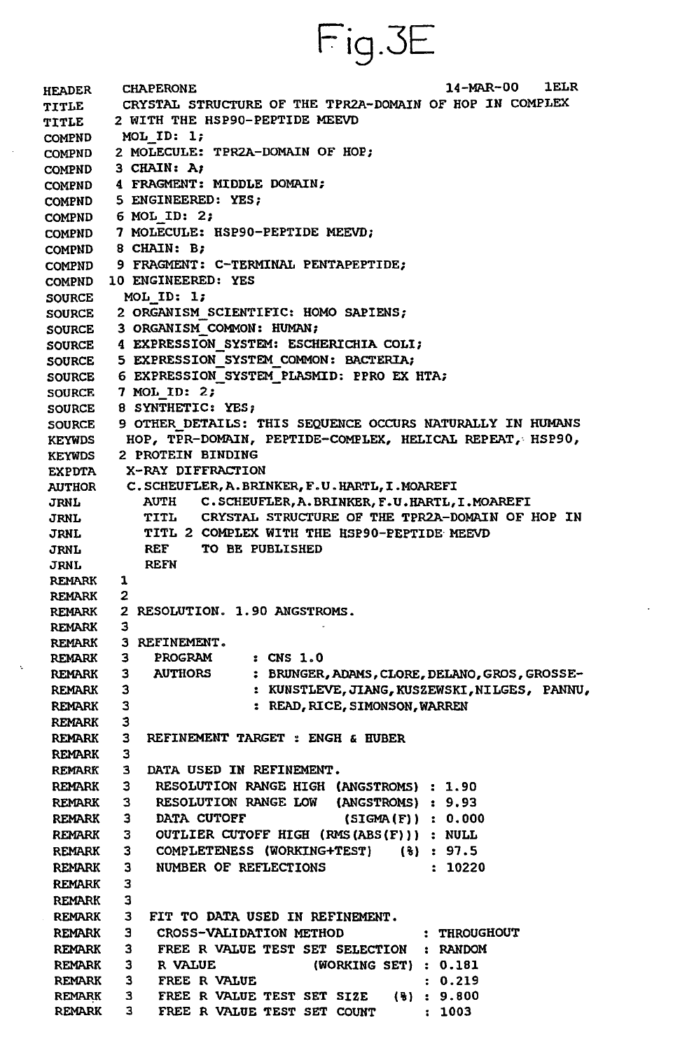

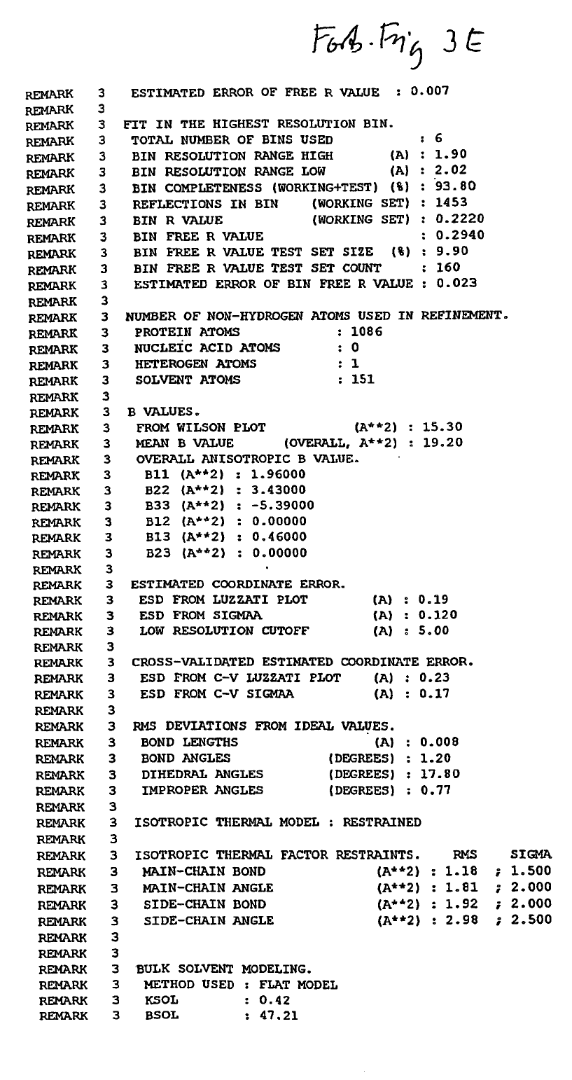

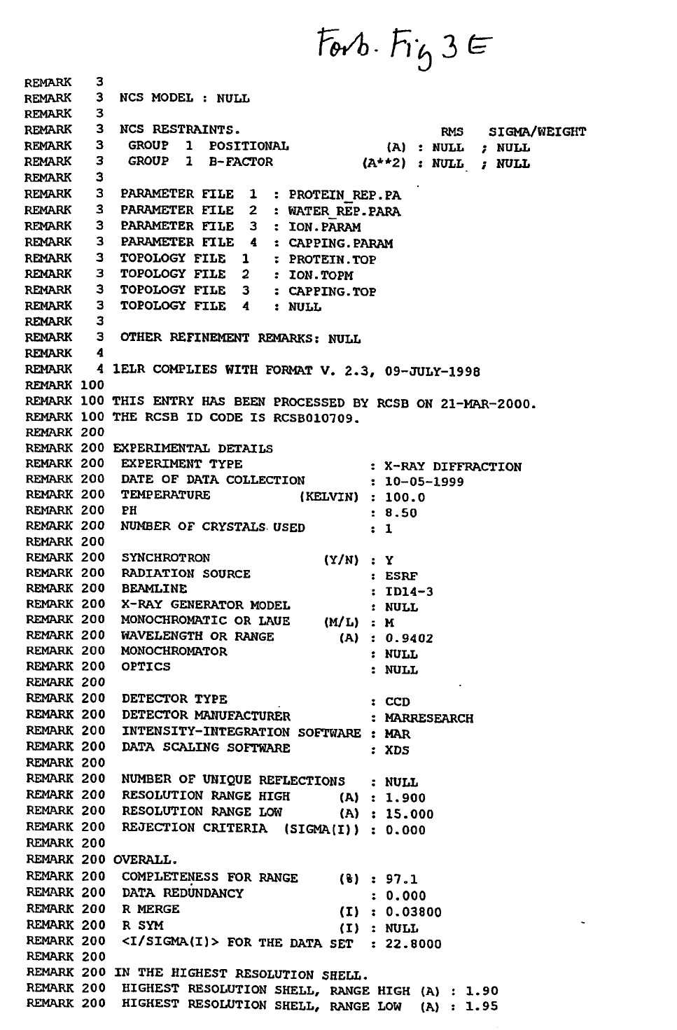

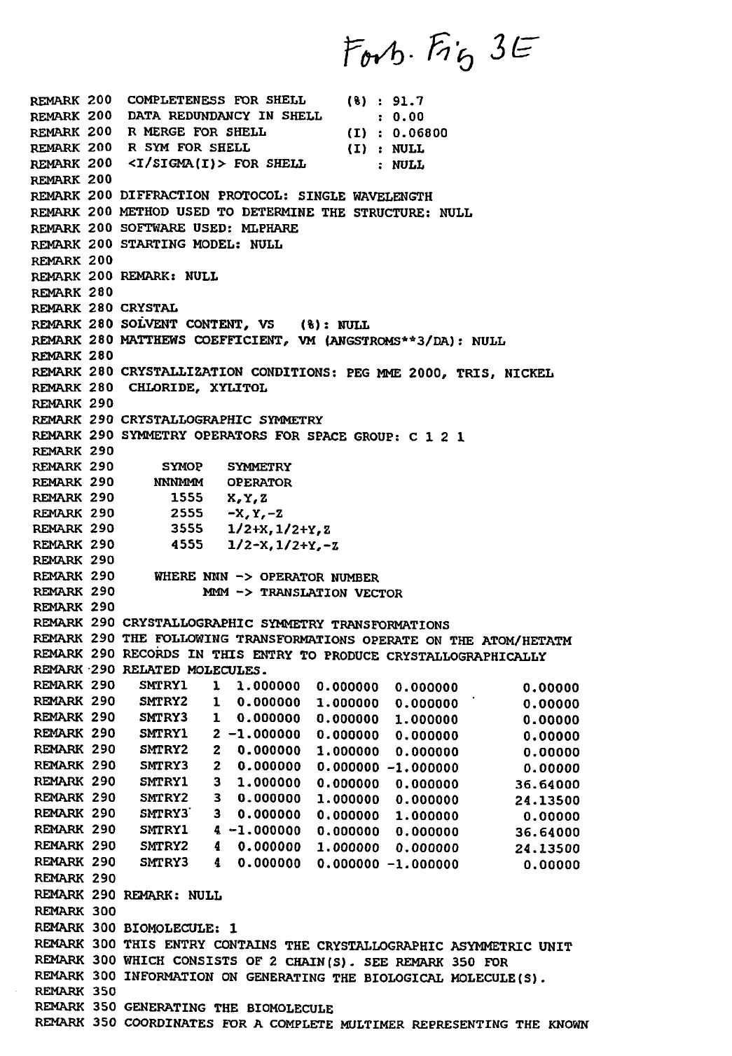

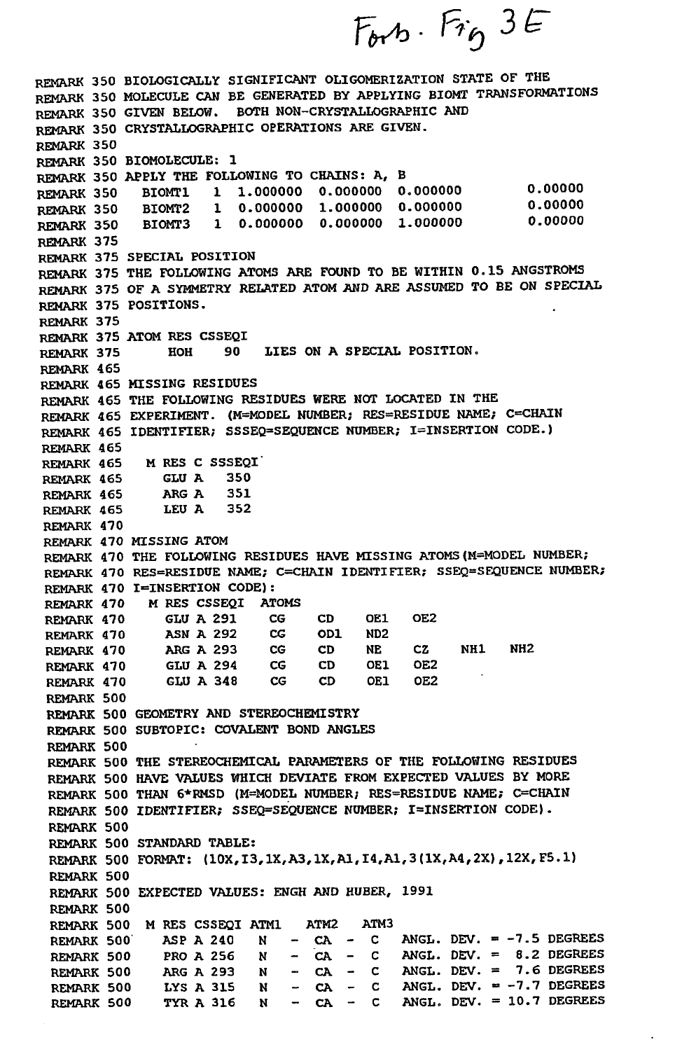

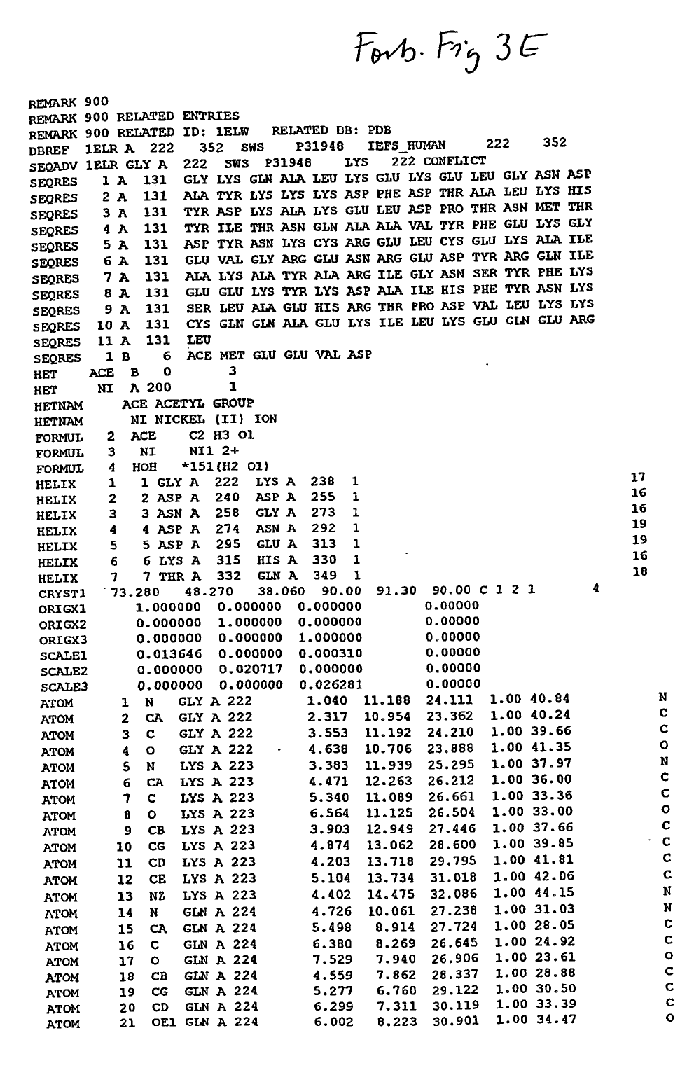

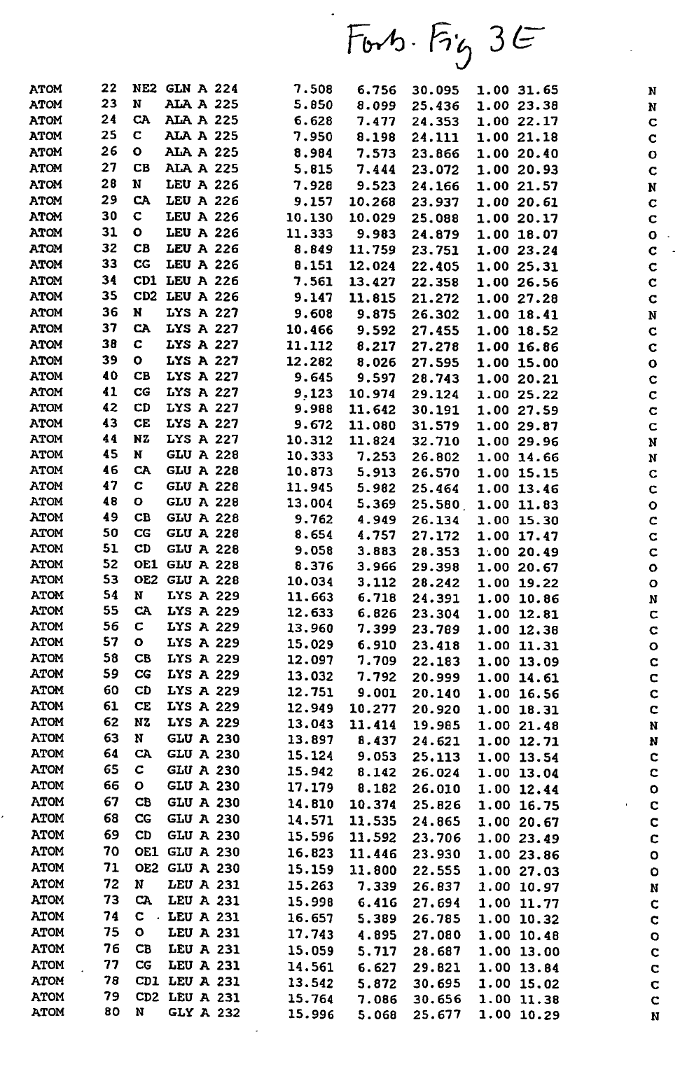

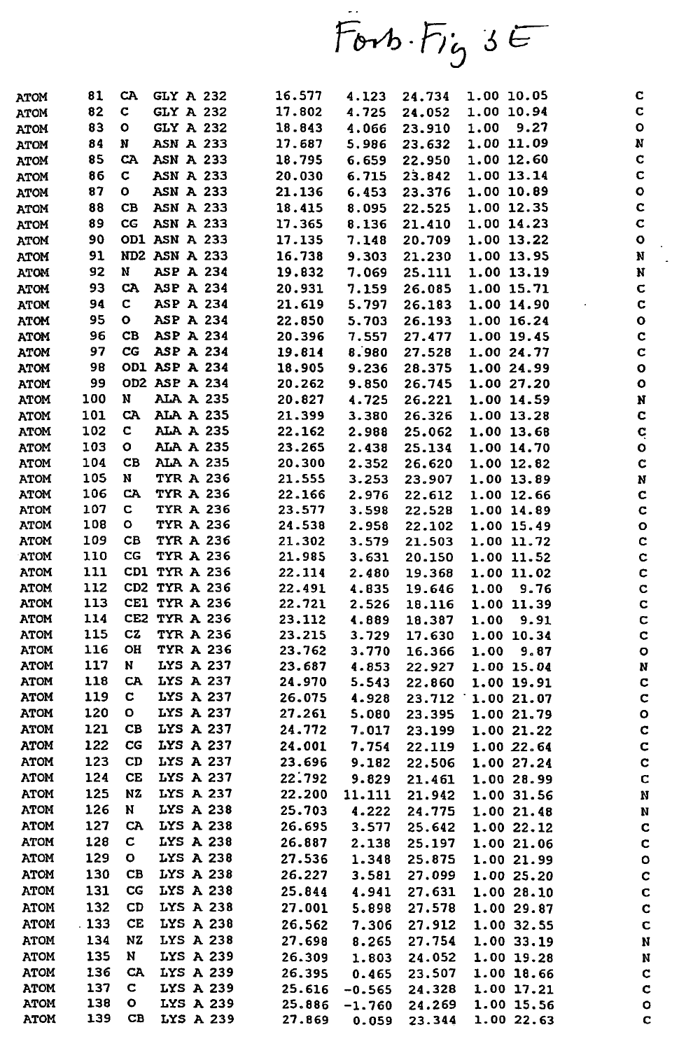

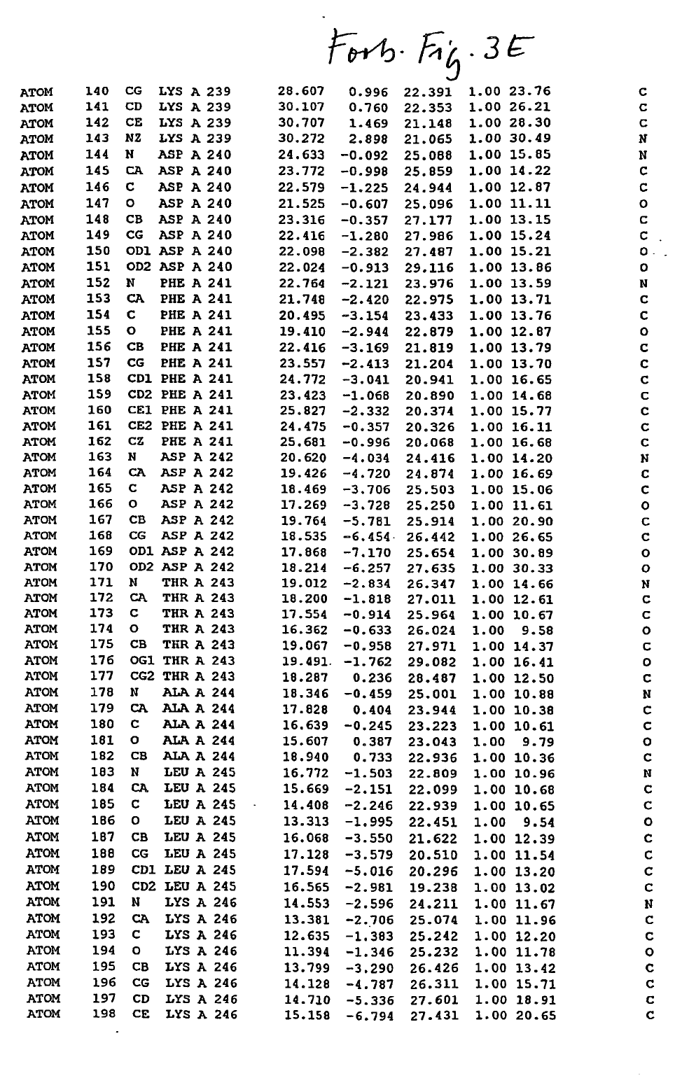

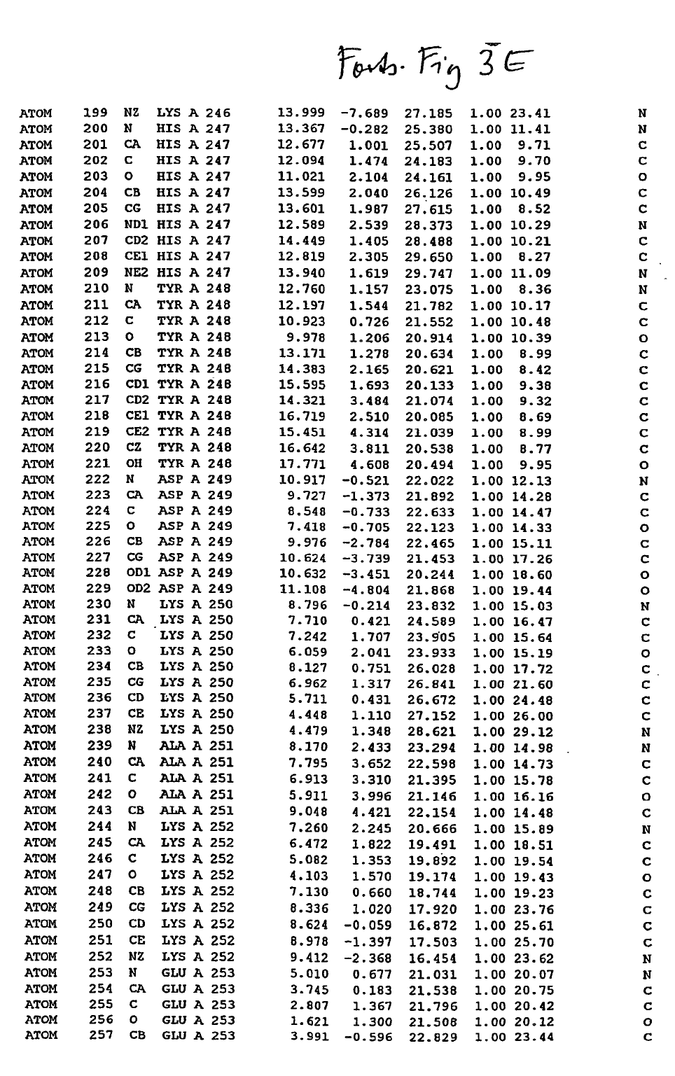

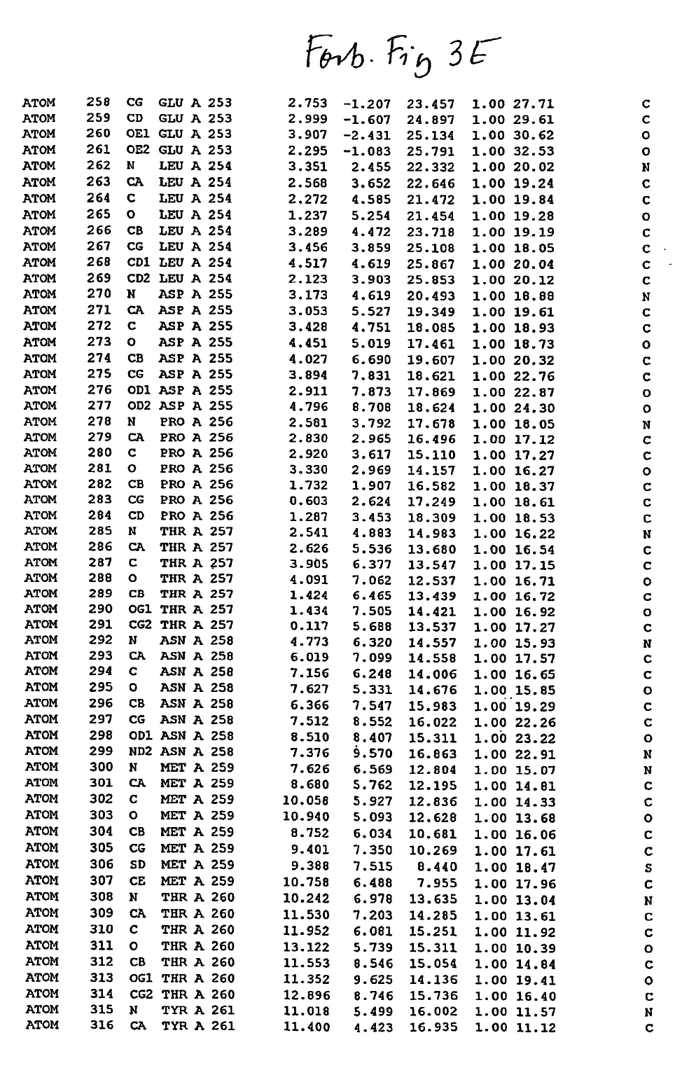

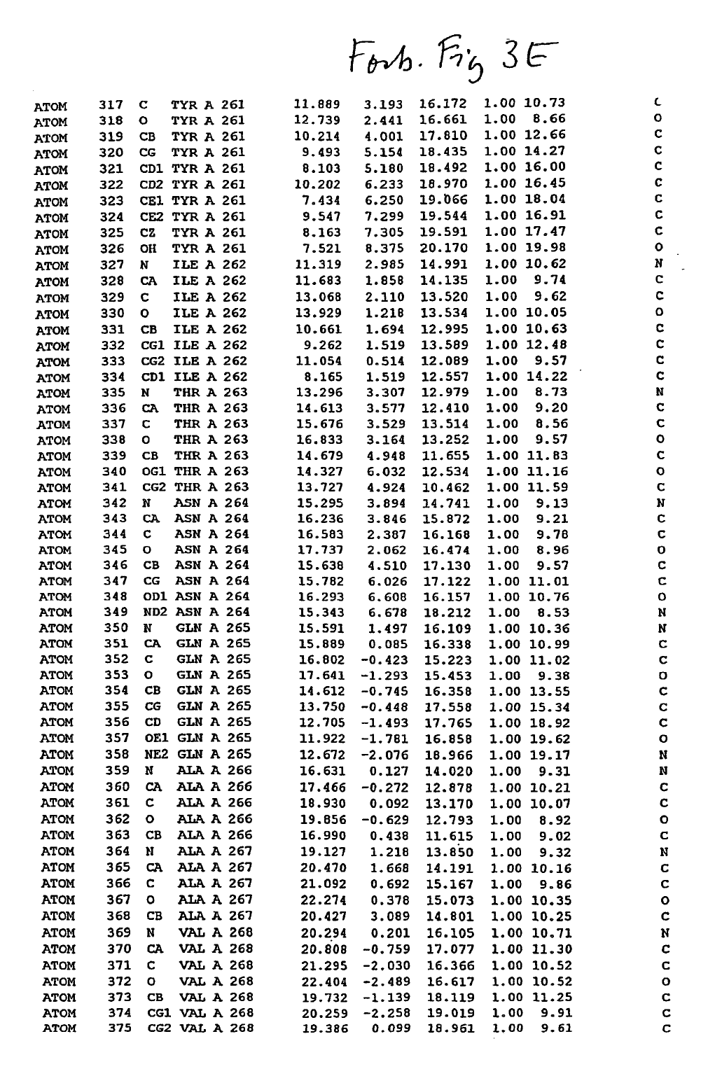

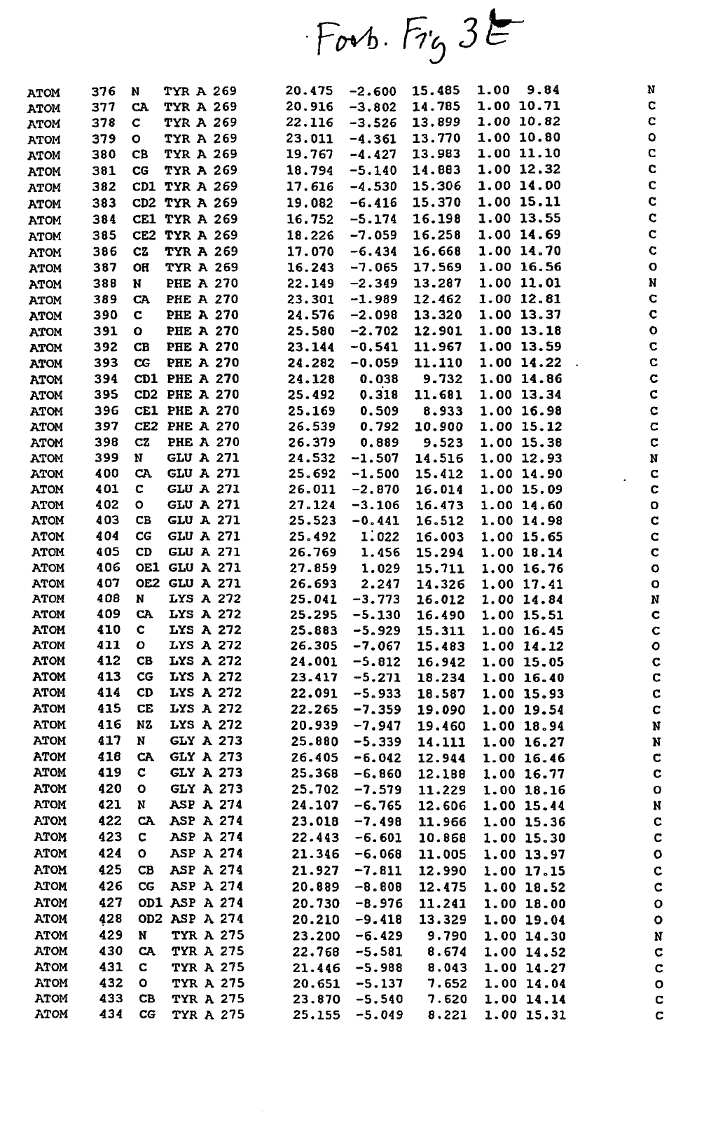

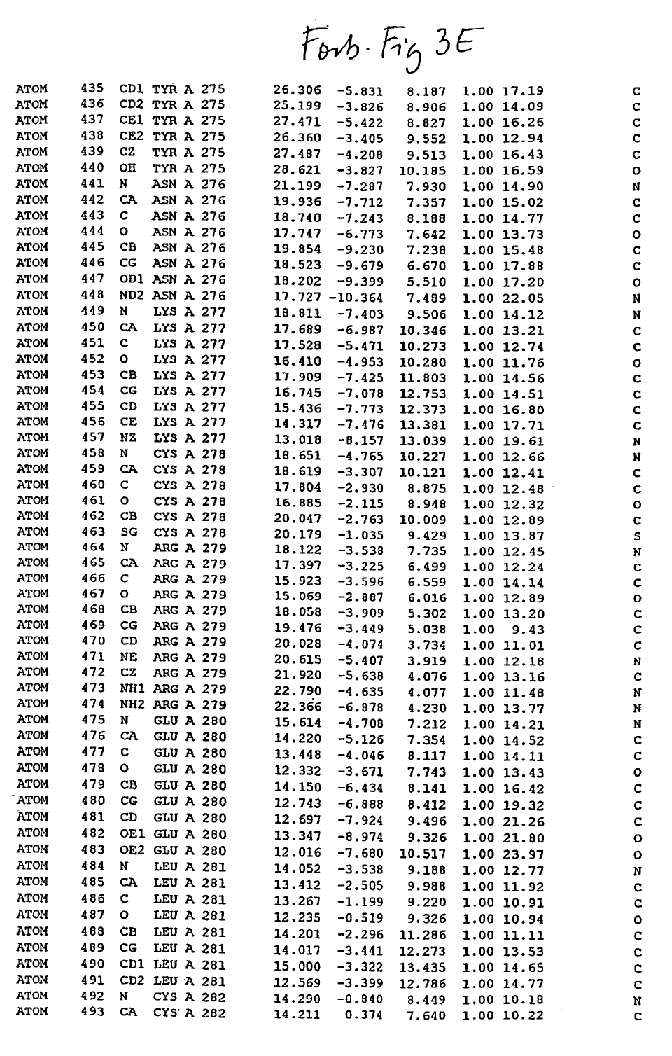

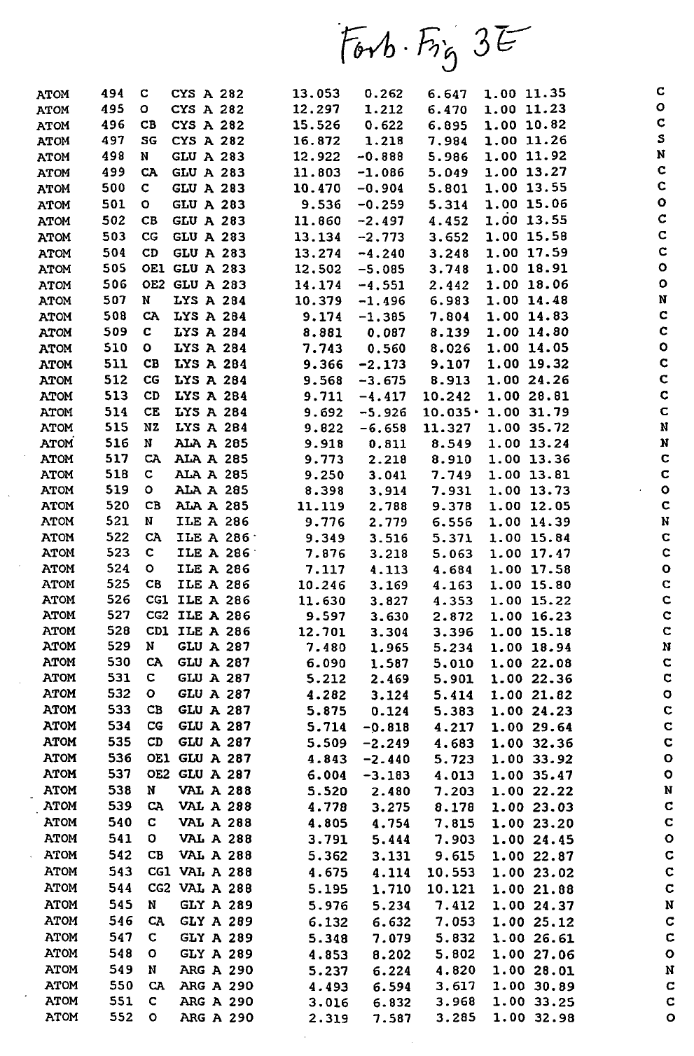

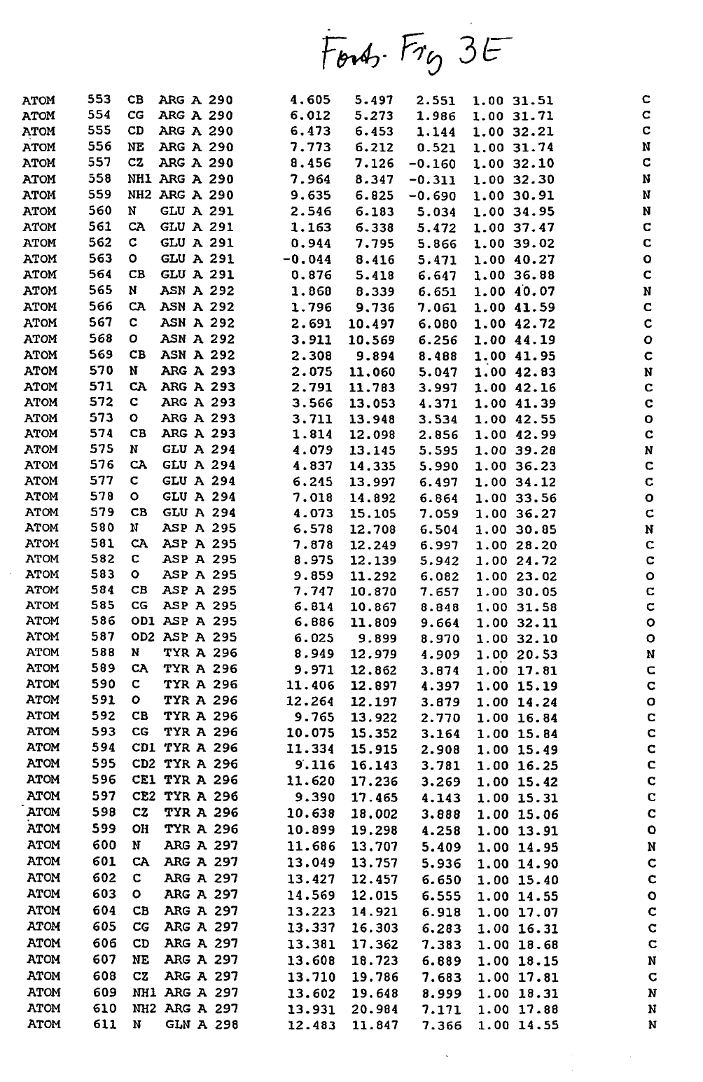

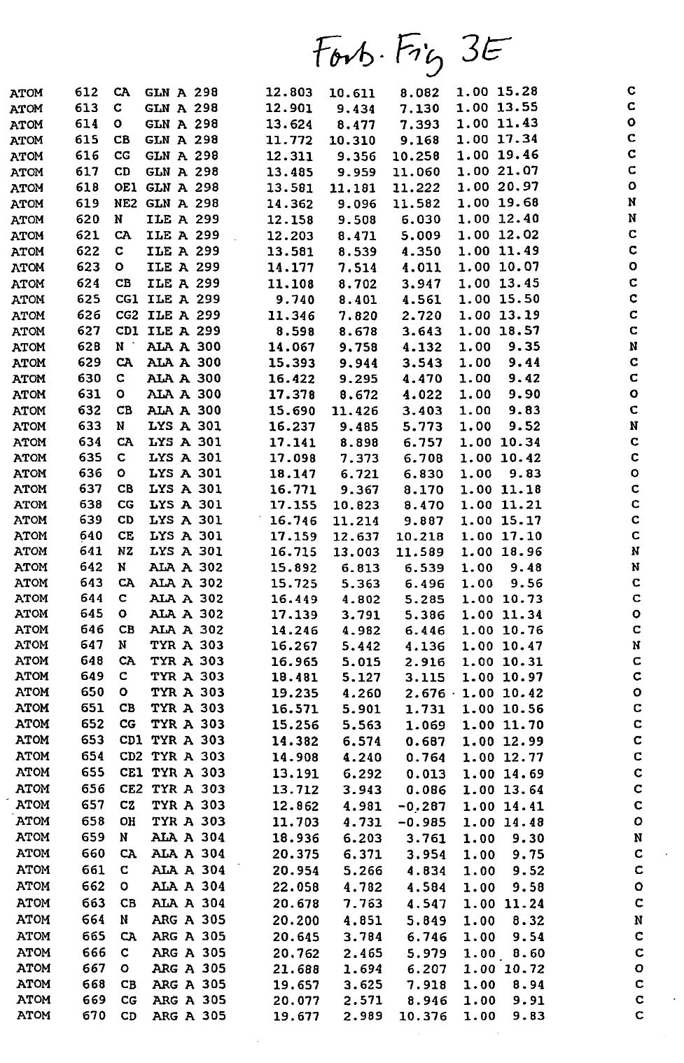

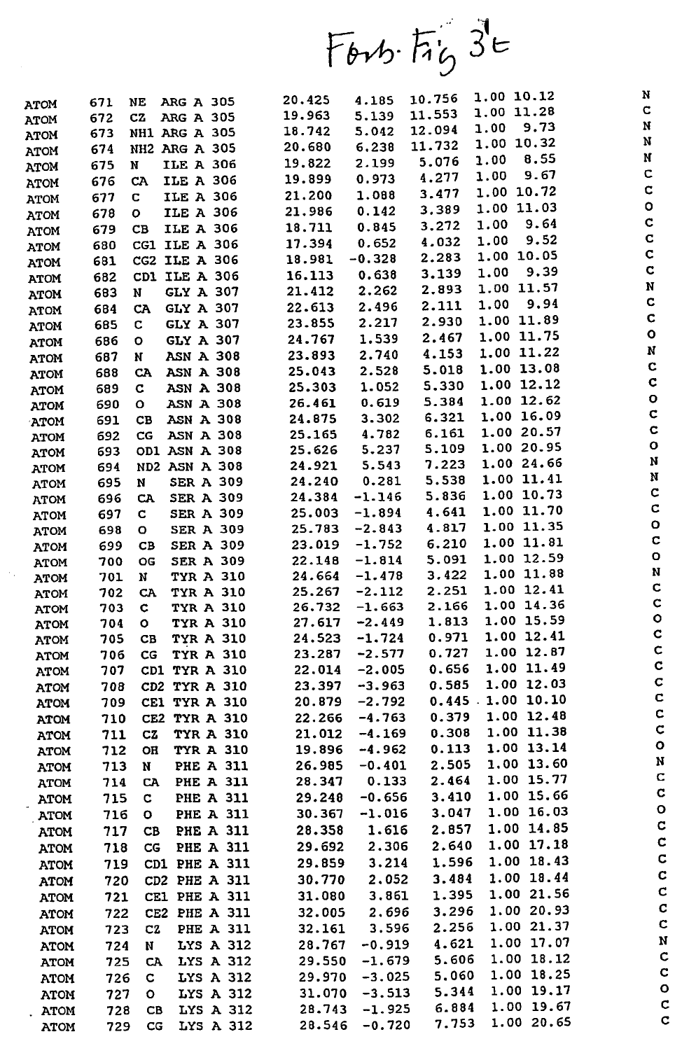

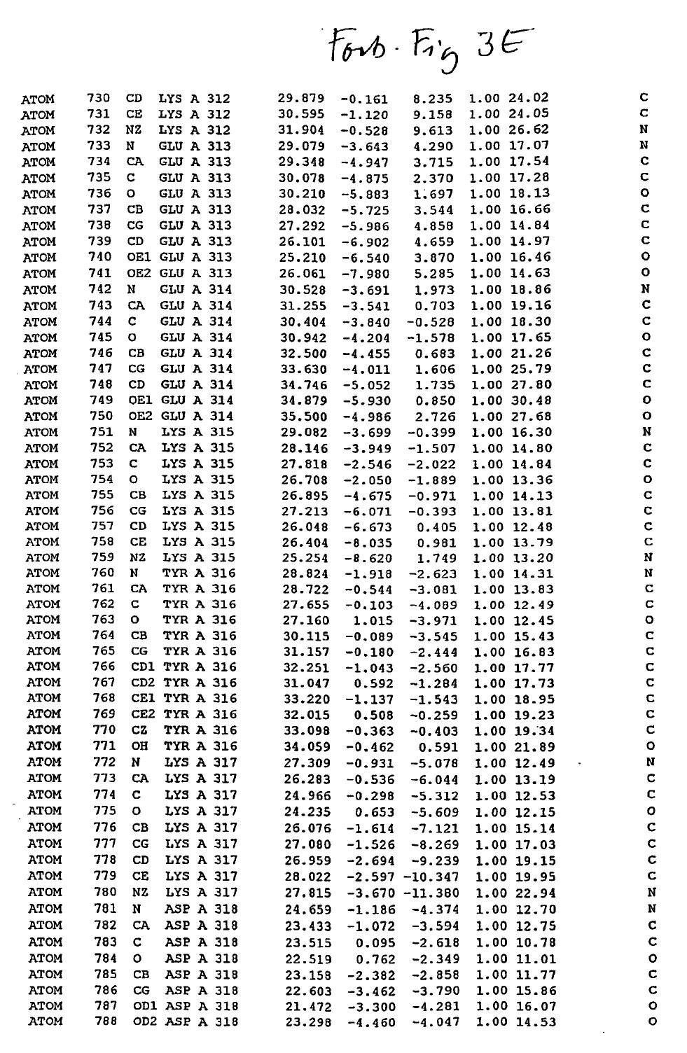

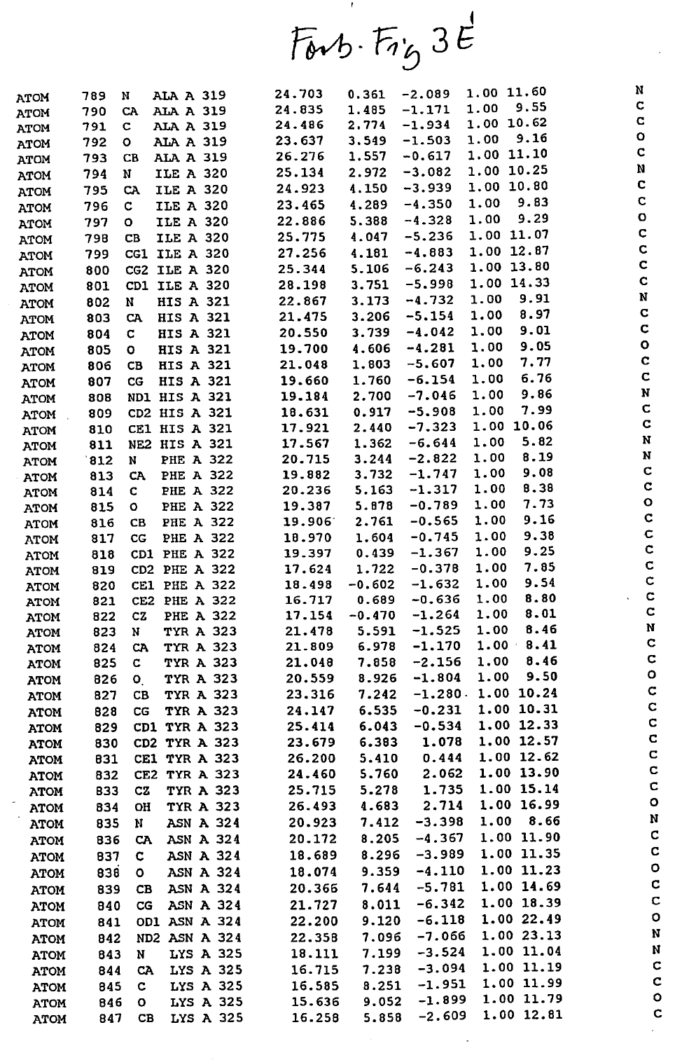

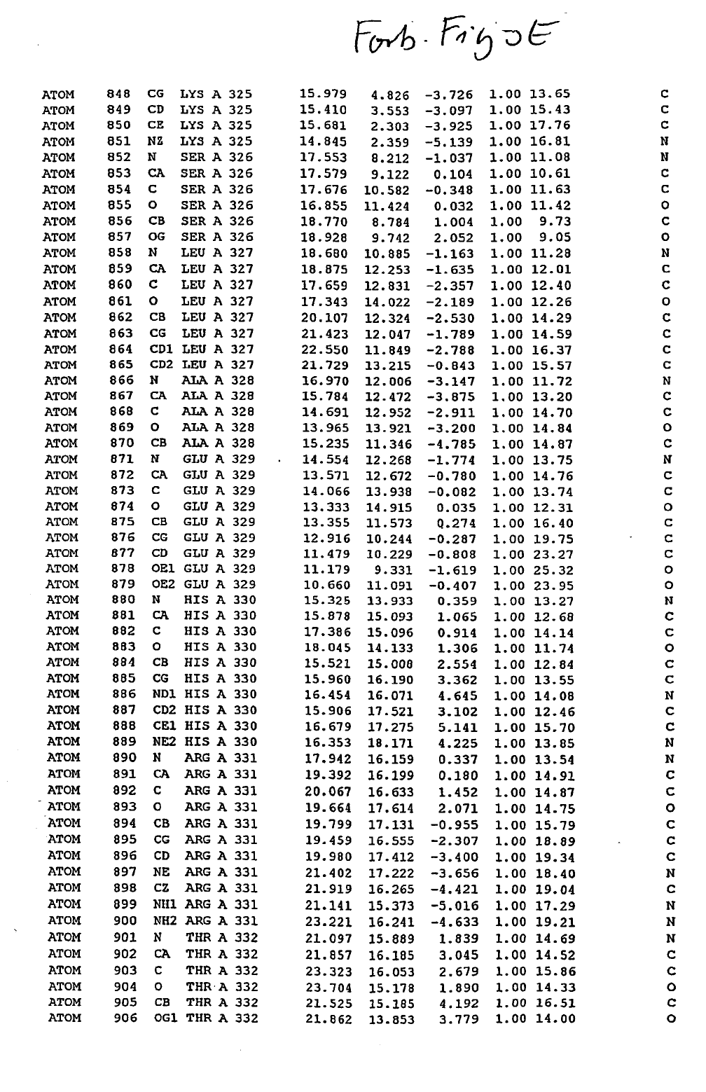

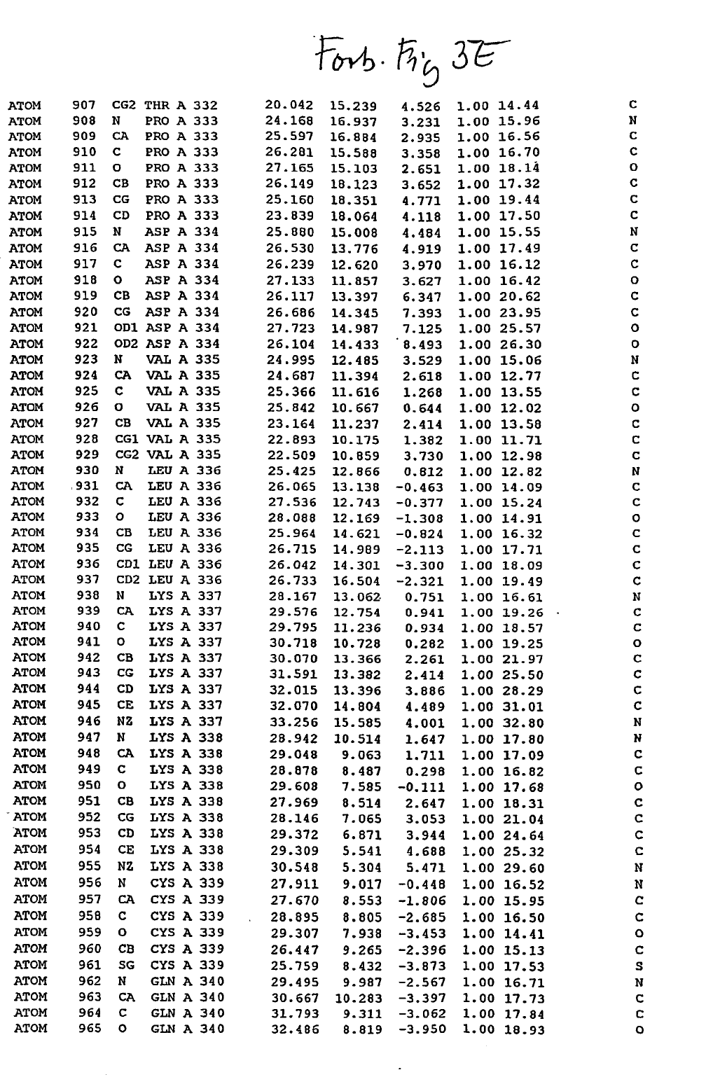

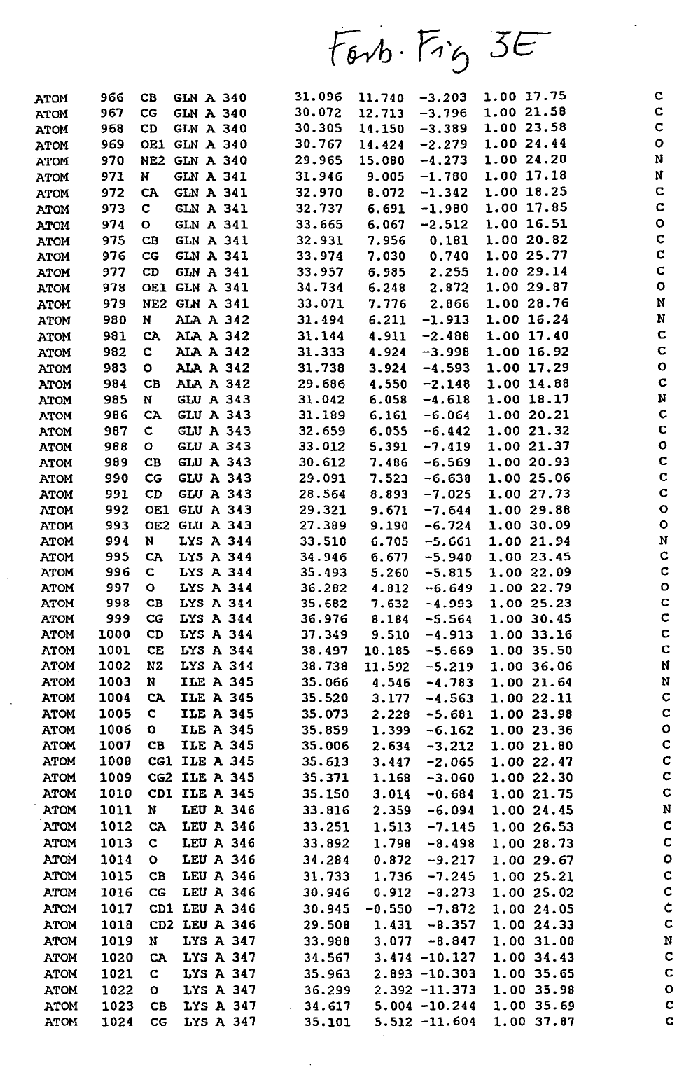

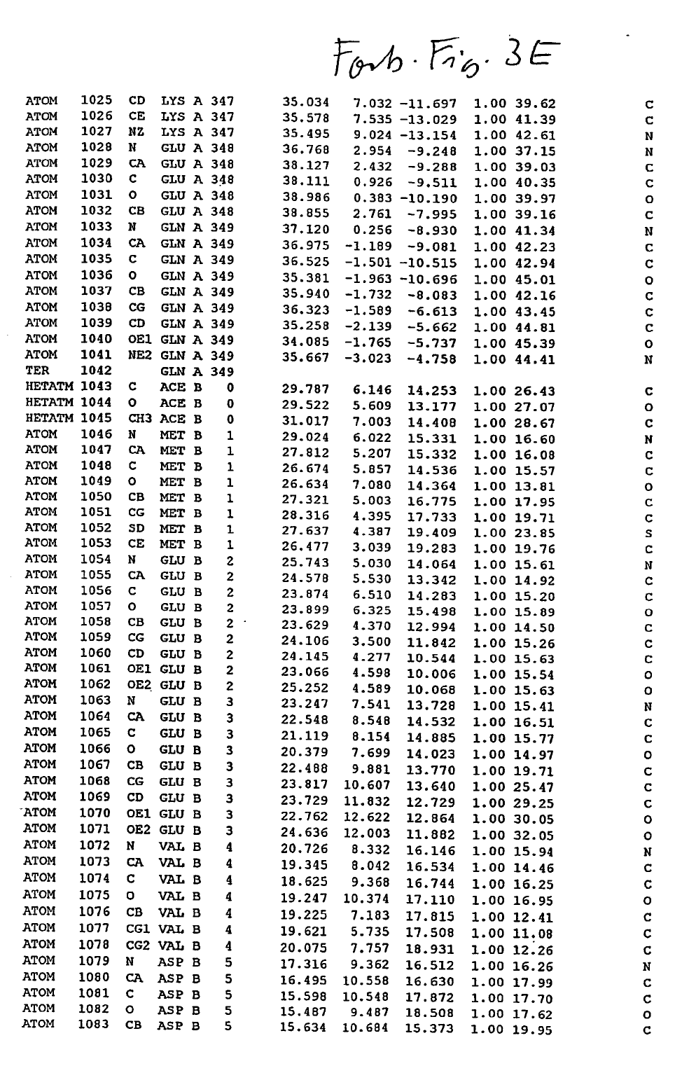

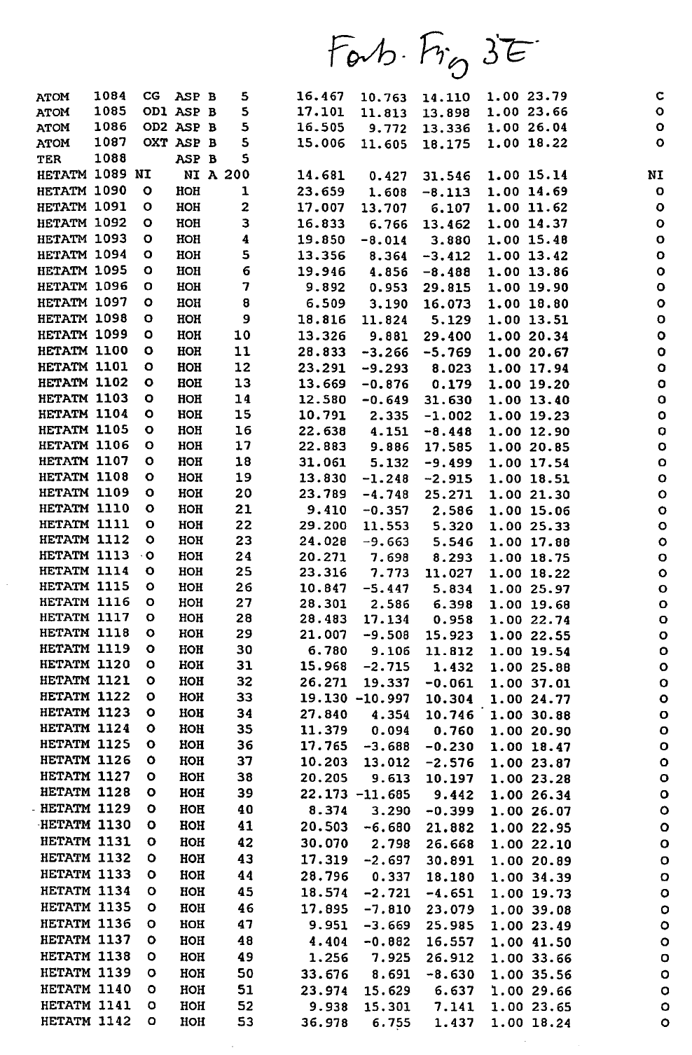

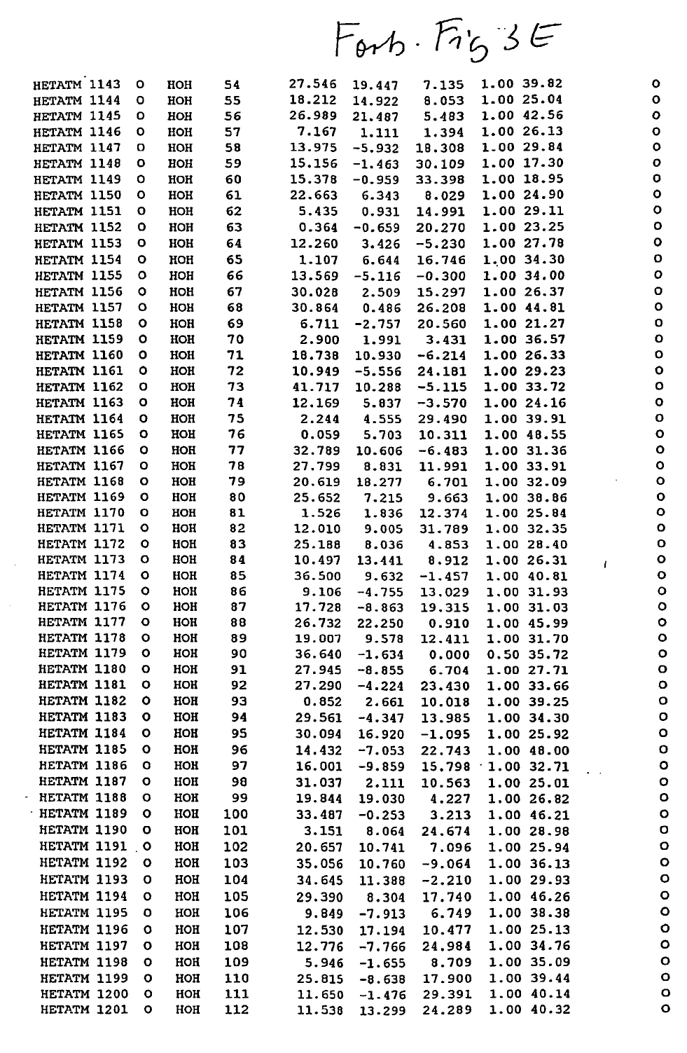

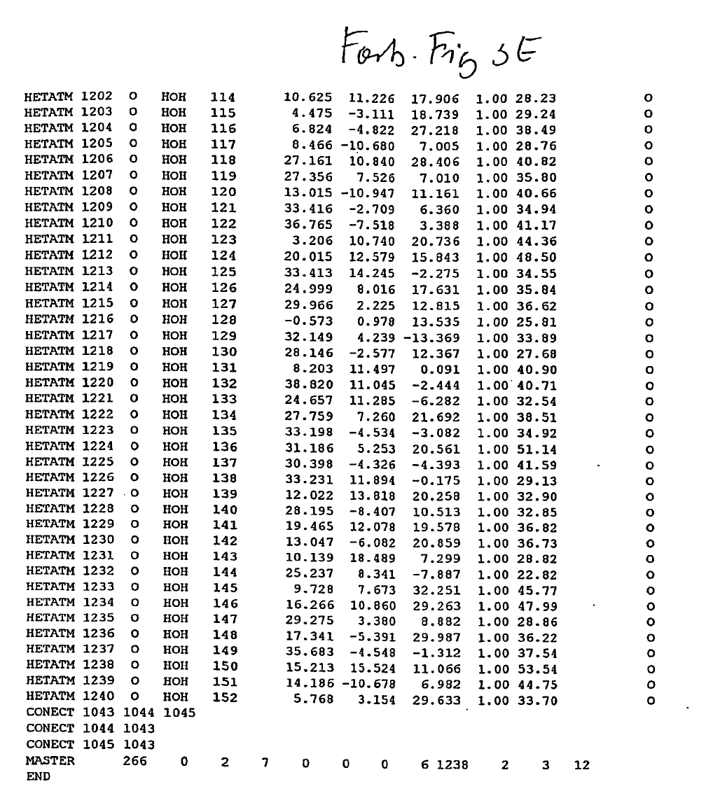

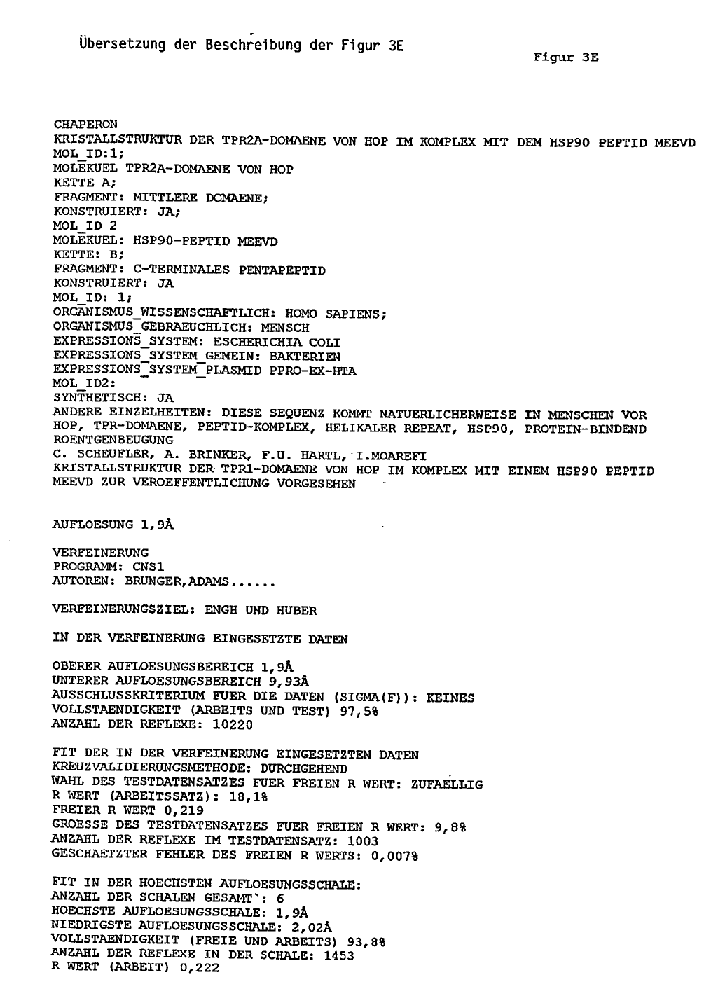

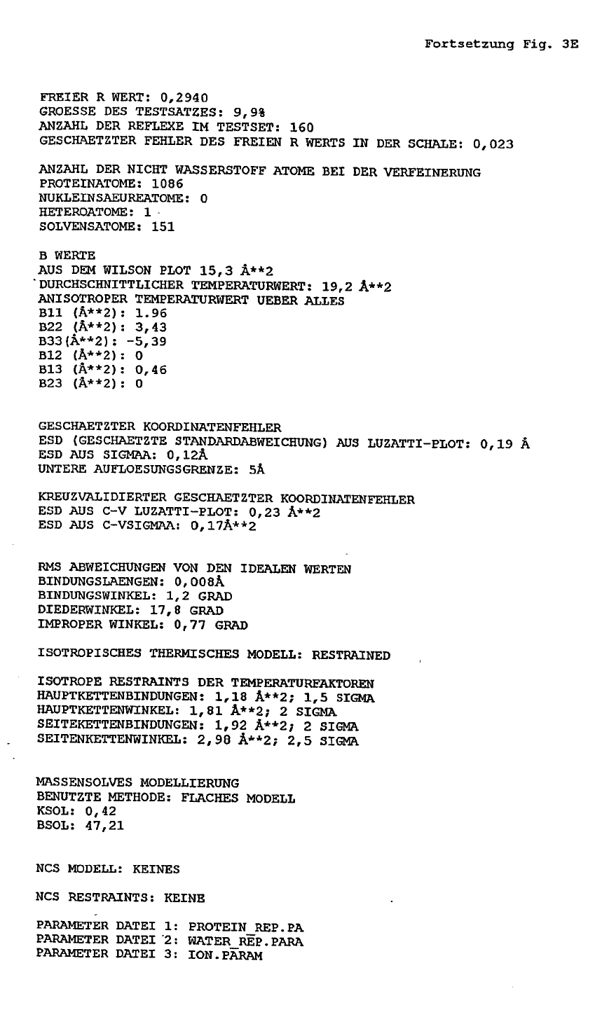

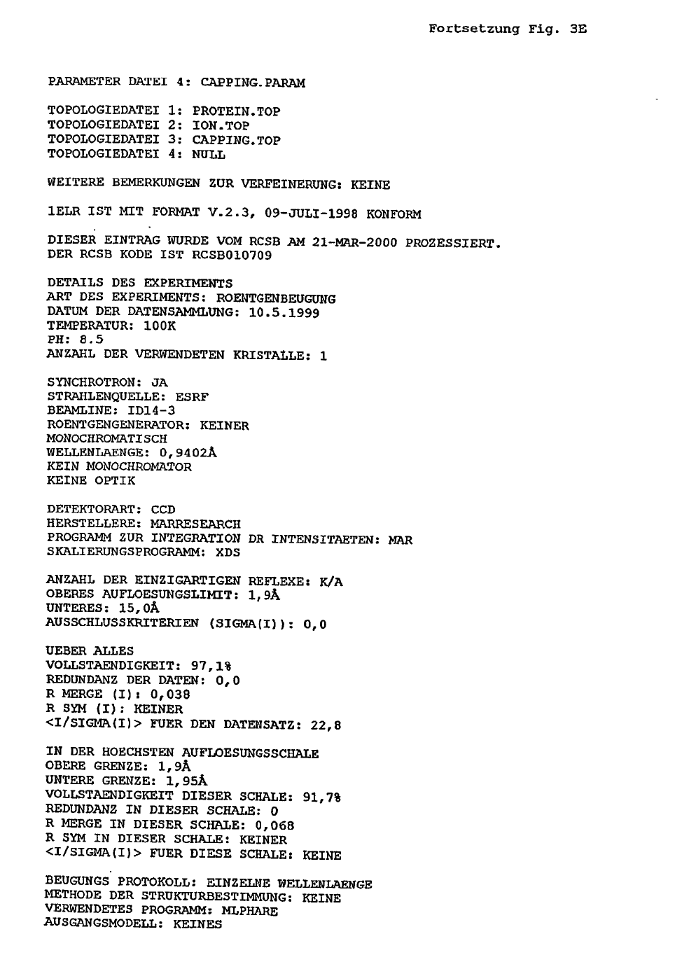

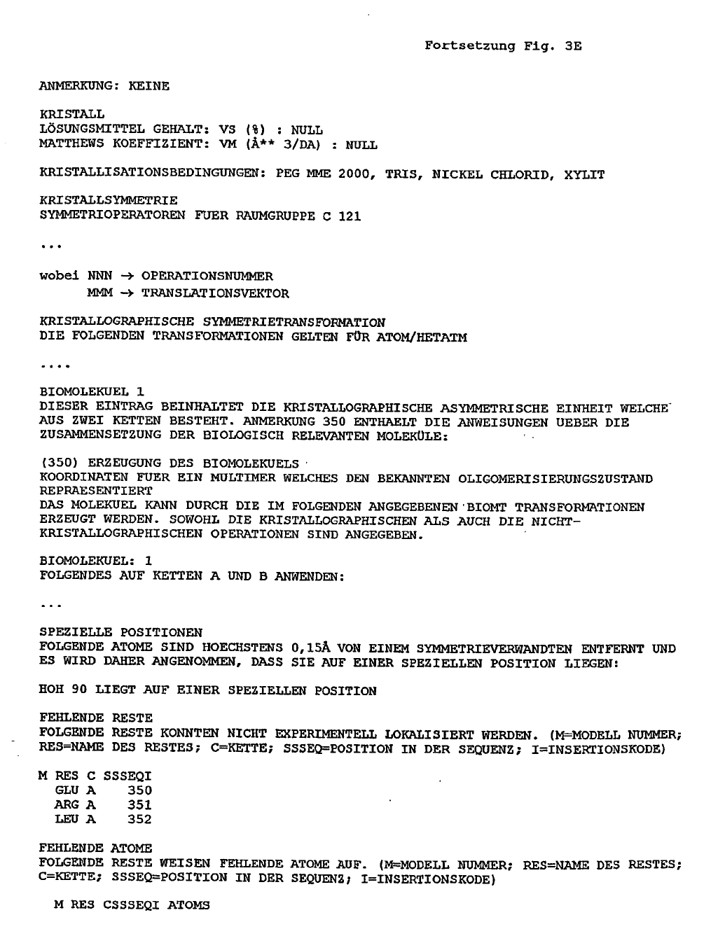

In accordance with claim 1 of the present invention, a 3D structure or space form of a polypeptide is claimed, where the 3D polypeptide contains at least one amino acid sequence of a TPR structure motif of a Hop protein or a derivative or a fragment of an amino acid sequence of such a TPR structure motif of a Hop protein. These amino acid sequences of a TPR structure motif of a Hop protein can, for example, be the amino acid sequences represented in FIG. 3A, marked consecutively as (1), (2), (3), (4), (5), (6), (7), (8), and (9). Such polypeptides, isolated or embedded or combined by other (“linker”) amino acid sequences, can show in random sequence, for example, the above amino acid sequences individually or in combination, eg. as a combination of the sequences (1), (4) and (7), or in any other combination of 2, 4, 5, 6, 7, 8, or 9 of the above sequences, for example (2) with (6), or (3) with (4), or also as combination of all nine above amino acid sequences.

Preferably, the 3D polypeptide will have at least one of the amino acid sequences of the TPR domains TPR1, TPR2A and/or TPR2B of a Hop protein, a section (or sections) or a derivative (or derivatives) of the above TPR domains. This means that the 3D polypeptide can contain several TPR structure motifs in form of the physiological TPR domains of a Hop protein. However, the TPR domain can also consist of combinations of TPR structure motifs not occurring physiologically. This includes, for example, also 3D polypeptides containing at least one TPR domain, where this at least one TPR domain can be made of, for example, three TPR structure motifs in artificial combination. This can either consist of combinations of already previously mentioned, and represented in FIG. 3A, Hop TPR structure motifs of different TPR domains of Hop proteins, or it can consist of a random combination of Hop TPR structure motifs having different TPR structure motifs of other proteins containing TPR domains, especially of co-chaperones containing TPR domains.

The term derivative of a TPR structure motif, or derivative of a TPR domain of a Hop protein, describes such primary sequences which mostly maintain the 3D structure, ie. the tertiary structure of the TPR domain or the TPR structure motif, as determined in accordance with FIGS. 3C, 3D, or 3E, and which only allow local structural deviations. Upon superimposing the tertiary structure of the primary sequence of the derivative with one of the original structures in accordance with FIGS. 3C, 3D, or 3E, a mean standard deviation for the vertical coordinates (rsdm) of less than 5 Å, especially less than 3 Å, and particularly less than 2 Å is preferred.

In this connection, especially such amino acids are called derivatives which only have conservative substitutions, ie. the exchange of, for example, polar with polar amino acids, or hydrophobic with hydrophobic amino acids (e.g. leucine with isoleucine or valine, or vice versa, or serine with threonine, or vice versa). Such sequences are interpreted to be a section or fragment of a TPR structure motif or a TPR domain of a Hop protein, which show, in contrast with the physiological amino acid sequence, deletions at the N- or C-terminal, or also intra-sequential deletions in the above sequences, which were, for example, also represented in FIGS. 3A and 3B. This consists typically of at least one deletion, where in each deletion preferably less than ten, especially less than five, and particularly preferably one or two amino acids can be deleted as opposed to the native sequence.

In a preferred embodiment of the present invention, a 3D structure of a polypeptide is claimed which contains at least one of the amino acid sequences (1), (2) or (3), as represented in FIG. 3B. In addition, such 3D structures of polypeptides are preferred, which contain at least one amino acid sequence of a TPR domain, where this sequence(s) correspond with those amino acid sequences which occur in Hop proteins of eukaryotic origin. They can also consist of derivatives, or rather sections or fragments, in the above sense of such sequences of Hop proteins of eukaryotic origin.

Especially preferred, however, are 3D structures of polypeptides which show one or more TPR structure motif/s or TPR domain/s of a Hop protein of human origin, or sections or derivatives of the same. Especially preferred are 3D structures of polypeptides, where the polypeptide shows the amino acid sequence of a eukaryotic, especially human, Hop protein, or also a derivative and/or a section of such a eukaryotic, preferably human, Hop protein.

Furthermore, such 3D structures are claimed, which show one polypeptide, containing at least one TPR structure motif of a protein containing a TPR domain, especially of a Hop protein, as well as containing also at least one other compound. This will typically consist of compounds which, being ligands, are able to bind to the polypeptide, so that the 3D structure represents a complex made of polypeptide and at least one other compound bound in a covalent or non-covalent manner. This compound, or ligand, can consist of a physiologically occurring molecule or also a non-physiologically occurring molecule. It may also be the case, if there is more than one ligand in the 3D structure, that combinations of physiologically or non-physiologically occurring molecules are contained in the 3D structure. Here, those ligands are preferred, which interact especially under physiological conditions with the polypeptide and show a high affinity binding behaviour (preferably K_{d}<30•μM). This can consist of, for example, chaperone proteins or sections or derivatives of the same, which interact with the respective polypeptides present in a 3D structure according to the invention, where the polypeptides contain TPR structure motifs or TPR domains of a protein containing TPR domains, especially a Hop protein.

Preferably, according to the invention, a 3D structure of a polypeptide is disclosed in combination with sections of physiological ligands. These consist of, for example, sequence sections or domains, which are relevant for the interaction with Hop proteins, of chaperone proteins such as Hsp70 and/or Hsp90. Typically, the ligand, preferably a physiological ligand, will bind to a sequence of a TPR structure motif contained in a 3D polypeptide according to the intervention, or, respectively, the ligand will interact with amino acids of an amino acid sequence of a TPR domain contained in the polypeptide. The ligand itself may be a polypeptide, an oligopeptide, a dipeptide, or a synthetically modified derivative of a poly-, oligo, or dipeptide, especially of sections of chaperone proteins which facilitate the interaction physiologically, but the ligand may also be a peptidomimetic or an organic-chemical molecule having a molecular weight of typically<5000.

Particularly preferred are 3D structures of polypeptides having one or more ligand/s, where the ligand contains a section of the C-terminal amino acid sequence of a chaperone protein, preferably of Hsp70 and/or Hsp90, or where the ligand consists of this section. Then, the ligand, if it does not feature the entire C-terminal domain of the chaperone protein, will typically comprise the 5 to 50, preferably 5 to 25, particularly preferred 5 to 12 C-terminal amino acid residues of such a chaperone protein. Particularly preferred are 3D structures which especially show a ligand bound to a TPR structure motif or to a TPR domain, where the ligands preferably have a binding affinity of K_{d}<50•μM, and thus typically block, at least in vitro, preferably also in vivo, the physiological function of Hop proteins. This will typically and functionally produce the inhibiting character of the ligand, when the binding location(s) of Hop proteins for the chaperone proteins are occupied with the ligand, so that the Hop protein loses its physiological adaptor function regarding the physiological interaction of Hsp70 and Hsp90. Thus, 3D structures of polypeptides are particularly preferred, which contain at least one Hop TPR structure motif or one TPR domain, where the ligand binds to the TPR structure motif and/or the TPR domain, and simultaneously acts as inhibitor of the interaction between the Hop and Hsp70 proteins and/or the Hop and Hsp90 proteins. Typically, the polypeptide will have binding locations for Hsp70 as well as for Hsp90 in the 3D structure according to the invention, and thus, it can also have ligands, which are bound to the Hsp70 binding location, as well as ligands, which are bound to the HSP90 binding location.

According to the invention, the 3D structure of a polypeptide of the type disclosed above, possibly in combination with one or more ligand/s, will consist of a 3D structure obtained by NMR structure analysis (Wüthrich, NMR Spectroscopy, 1986), or of a crystal structure. The crystal structure is obtained as a result of crystallisation of the polypeptide and possibly other components, for example at least one ligand, by means of subsequent X-ray crystallographic structure determination (Stout and Jensen, X-RAY Structure Determination, Wiley, 1989; the disclosure in Stout and Jensen is entirely included in the present disclosure regarding the process of X-ray crystallographic experiments).

Crystal structures according to the invention distinguish themselves due to the fact that, being a three dimensional structure characterised by structure coordinates for each atom making up the structure, they are part of a symmetrical arrangement in a crystal. Here, it is preferred that a crystal structure according to the invention, which contains at least one polypeptide having at least one TPR structure motif or preferably at least one TPR domain, particularly of a Hop protein, shows, upon superimposing with the structure coordinates listed in FIGS. 3C, 3D, or 3E, a mean standard deviation (rmsd) of less than 2.5 Å, preferably less than 2 Å, for the at least one TPR structure motif or the at least one TPR domain involved in the binding reaction.

If the 3D structure is a crystal structure, the crystal structure typically contains additional metal ions besides the atoms of the polypeptide, or possibly the ligand/s (except hydrogen atoms), for example alkaline-earth or alkaline metal ions, especially calcium ions, but also heavy metal ions suitable for phase determination, such as gold, nickel or mercury ions.

Particularly preferred are 3D structures of polypeptides, which are present in crystal structure, when the crystal structure of the polypeptide contains at least one TPR structure motif of a Hop protein, especially one of the amino acid sequences marked as sequences (1), (2), (4), (5), (7), and/or (8) according to FIG. 3A, (or their derivatives or fragments), where these sequences show structure coordinates as provided for the sequences (1), (4) and (7) in FIGS. 3C or 3D, and for the sequences (2), (5) and (8) in FIG. 3E. FIGS. 3C and 3D represent the structure coordinates for the TPR domain TPR1 of Hop, where FIG. 3E lists the structure coordinates of the TPR domain TPR2A of Hop. Due to their amino acid sequences, the above mentioned primary sequences (1), (2), (4), (5), (7), and (8) according to FIG. 3A can readily be assigned to their respective tertiary structures listed in FIGS. 3C, 3D, and 3E. To do so, the structure coordinates of the atoms present in the above mentioned sequences can be gathered from FIGS. 3C, 3D, and 3E. Each list of atoms in FIGS. 3C, 3D, and 3D is sequential, i.e. amino acid by amino acid from N- to C-terminal.

Finally, 3D structures of a polypeptide as a crystal structure are particularly preferred if the polypeptide being a crystal structure contains at least one TPR domain of a Hop protein (structure of the sequences (1) and (2) according to FIG. 3B) with all respective structure coordinates, as represented in FIG. 3C and FIG. 3D for the TPR domain TPR1, and in FIG. 3E for the TPR domain TPR2A of Hop.

An additional subject matter of the present invention are crystals showing 3D or crystal structures, as claimed in claims 1 to 22, arranged according to the laws of symmetry. This includes crystals of all such crystal structures, which are disclosed according to the present invention. Thus, according to the present invention, crystals are claimed which are made of unit cells where the asymmetric unit in the unit cell of the crystal shows at least one other compound, and where in addition, the polypeptide in the crystal assumes a 3D structure as a crystal structure, as disclosed above.

Preferably, the crystal will show a space group, which is monocline, tetragonal, orthorhombic, cubic, tricline, hexagonal, or triagonal/rhombohedral. They can consist of native crystals, derivative crystals or also co-crystals. Typically, the space group of the crystal, showing a 3D crystal structure according to the invention, will be the space group P2₁, C2 or P4₁. Basically, however, 3D structures (crystal structures) according to the invention can occur in all possible protein-crystallographic space groups. Particularly preferred are such crystals according to the invention, whose unit cell shows cell constants of approximately a=31.2 Å, b=43.8 Å, c=38.3 Å, and β1=101.8°, or a=75.5 Å, and c=42.9 Å.

An additional subject matter of the present invention are the methods for the manufacturing of a crystal with unit cells, containing in the asymmetric unit at least one 3D structure of the type according to the invention, i.e. at least one 3D structure of a polypeptide and possibly at least one additional compound, where (a) the polypeptide is applied as a coating in an extrusion system, (b) the polypeptide coating is cleaned and re-concentrated, (c) the polypeptide concentrate obtained according to (b) is dissolved in a suitable buffer system, possibly after adding at least one additional compound, and (d) the crystallisation is induced by, for example, means of a vapour diffusion method. Also with regards to the possible crystallisation methods, the details in Stout and Jensen (see above) are entirely included in the present disclosure.

As an additional subject matter of invention, compounds are disclosed which, being ligands, can bind to TPR structure motifs or TPR domains, each of which can again be part of longer polypeptide chains, preferably to TPR structure motifs or domains of Hop proteins. For example, PP5, FKBP51, FKBO52, Cyp40, TOM34, TOM70, CNS1-sc, TTC1, TTC2, TTC3, TTC4, IRSP, SGT, or KIAA0719 can also come into consideration as proteins with TPR domains to which ligands, according to the invention, can bind. Compounds according to the invention do form non-covalent interactions with the main chain and/or the side chains of amino acids, which are part of a TPR domain, preferably of a TPR domain of a Hop protein, or of one of the above mentioned proteins. Preferably, through this binding of the ligand, the physiological adaptor function of the Hop protein, which brings the chaperone proteins Hsp70 and Hsp90 into direct contact, is blocked. Thus, the compound, in a preferred embodiment, binds the 3D structure of a polypeptide according to one of the claims 1 to 22, or the 3D structure of a polypeptide, which has at least any one TPR structure motif, in such a way that the physiological binding of chaperone proteins, especially Hsp70 and/or Hsp90, to the TPR structure motif or to the TPR structure domain, preferably of a Hop protein, is blocked. Here, the ligands will interact preferably with the amino acids Lys8, Asn12, Asn43, Lys73 and Arg77 (in accordance with the method of counting in the Hop protein), or with the conserved amino acids, which are located at the respective positions, of other TPR domain proteins (e.g. FIG. 3). As a particular preference, the ligands will form part of the interactions with the TPR domain, or all of the interactions, which are represented in a diagram in FIGS. 4A and 4B for each of the bound peptides. In particular, the ligands will form those hydrogen bridge bindings, hydrophobe contacts, van-der-Waals interactions, or electrostatic interactions, which are also formed by the peptides having the complementary amino acid residues of the TPR domains. For example, a ligand according to the invention will preferably have, at equivalent steric positions, equivalent functional groups with the bound peptides. In particular, a ligand in accordance with the ligand, will also have a double-carboxylate-function in order to be able to be anchored in the TPR domain. I connection with the potential interactions of the ligand of a TPR domain, reference is made to the description of FIG. 4, and to the representation of the results in the description of the results of the embodiments. A ligand according to the invention will form at least some, possibly all of the interactions, with the TPR domain, as described in this figure so that a binding affinity Kd of less than 100•M, preferably less than 50•M, particularly preferred of less than 20•M is ensured.

Thus, as a whole, a ligand according to the invention, will have a structural design, which is complementary to the binding area of a TPR domain, preferably of a Hop domain, especially to the structural requirements given in FIGS. 3C, 3D, or 3E. These ligands, preferably having an inhibitor function for a chaperone or co-chaperone function, can be modified or unmodified di-, oligo-, or polypeptides. A peptidomimetic of a di- or oligopeptide is also possible. The peptidomimetics can preferably be such compounds whose backbone does not show amid-type bindings, but other chemical bridges in order to prevent proteolytic splitting. An inhibitor compound in accordance with the invention can, for example, be a C-terminal section of a chaperone protein, preferably Hsp70 or Hsp90. Particularly preferred are peptides containing the last 50, more preferred containing the last 30, even more preferred containing the last 20, and even more preferred containing the last 8 or 10, and most preferred containing the last 5 C-terminal amino acids of Hsp70 or Hsp90, where this peptide sequence might be chemically modified, e.g. by modifications of the peptide backbone. This means that such a modified or unmodified peptide according to the invention can contain the amino acids EEVD at the C-terminal.

Particularly preferred as inhibitors according to the invention are modified or unmodified oligopeptides, preferably containing the amino acid sequences GPXIEEVD (single letter code) or SXMEEVD, where X stands for any, naturally occurring amino acid.

The present invention comprises especially those ligands, which have the ability to bind to a 3D or crystal structure, represented by the structure coordinates according to FIGS. 3C, 3D or 3E.

An additional subject matter of the present invention are the methods of identification of a compound, which has the characteristic to act as an inhibitor of the interaction between Hop protein and the chaperone protein, especially Hsp70 or Hsp90, particularly human Hsp70 and/or Hsp90. Such a method is particularly preferred, if the compound with ligand function binds to a structure area of a TPR domain, especially in the area of the active binding centre. Such a method is characterised by the fact that (a) a crystal structure is obtained according to claims 1 to 22, where the crystal structure is present in the form of its structure coordinates, that (b) the structure coordinates of the crystal structure are represented in three dimensions, and that (c) the steric properties and/or functional groups of a compound with ligand function are selected in such a manner that interactions between the compound and the main and/or side chains of the polypeptide, which forms the active centre, become possible. According to these interactions, suitable ligands according to the invention are determined, especially suitable inhibiting ligands, blocking the interaction between Hop protein and Hsp70 and/or Hsp90.

The representation of the structure coordinates of a crystal structure according to the invention preferably takes place by means of graphic representation on a computer screen using relevant computer programs. In relation to potential ligands, suitable ligands with the relevant chemical and/or steric properties can be, dependent on the operator's experience, identified in a non-automated manner, designed on the screen and, finally, their binding behaviour can be simulated by making use of the complementary arrangement of the main and side chains of the crystal structure, e.g. in the binding area of a TPR domain, e.g. of a Hop protein or a structurally related protein.

Preferably, however, the selection of suitable ligands is carried out in an automated manner by searching computer databases containing a multitude of compounds. The search is based on the previously carried out characterisation of geometric, chemical and/or physical properties for the desired ligands, e.g. compounds with structural and/or functional similarity to the claimed compounds (claims 28 to 37). Databases to be searched contain naturally occurring as well as synthetic compounds. For example, the compounds stored in CCDC (Cambridge Crystal Data Centre, 12 Union Road, Cambridge, GB) can be used for such a search. Also, the databases available through Tripos (see the above reference), i.e. Aldrich, Maybridge, Derwent World Drug Index, NCI and/or Chapman & Hall can be searched. The following computer programs can be applied for such a search: especially the “Unity” program, “FLEX-X” (Rarey et al. J. Mol. Biol. 261, 470-489, 1996), “Cscore” (Jones et al., J. Mol. Biol. 245, 43, 1995) of the Sybyl Base Environment of the Tripos program package.

In the following section, the application of a method, according to the invention, for the computer-based identification of potential ligands is described in more detail. First, the desired binding area of a ligand in a crystal structure according to the invention needs to be defined. The ligand will typically consist of a ligand with inhibiting properties, but activators are also possible. The binding area is characterised by relevant parameters, e.g. distances between atoms, hydrogen bridge binding potentials, hydrophobe areas and/or charges, and on this basis, boundary conditions for the chemical, physical and/or geometrical properties of the ligand are defined. At the binding process, at least one of the previously specified amino acids (see claim 31) are preferably involved, especially amino acids with the above mentioned side chains. Therefore, regarding an existing method according to the invention for the identification of compounds, complete content reference is made to the previous disclosure regarding the “compound” subject matter of the invention according to claims 28 to 37. Subsequently, computer programs identify such compounds, which fulfil the previously introduced conditions, in relevant databases. Here, the use of the program package Sybil Base (Tripos, 1699 South Hanley Road, St.Louis, Mo., USA) is especially preferred. Here, it is especially preferred that the database to be searched provides compounds, giving details regarding their respective 3D structures. If this is not the case, a computer program will be applied for a method according to the invention, preferably in step (d) of the method, which first of all calculates their three-dimensional structure prior to the review (e.g. “CONCORD” program from the Sybyl environment by Tripos Inc.), if the provided boundary conditions of a ligand are fulfilled.

Typically, in procedure step (e), the interaction potential between an identified compound and the desired binding area in a crystal structure is determined, e.g. within the scope of an automated search for a compound in a computer data base. A procedure according to the invention is particularly preferred, if it helps identify compounds, which are supposed to bind to a crystal structure with the structure coordinates of FIGS. 3C, 3D or 3E. The intensity of an interaction, determined with the help of procedure step (e), between a compound obtained from a database and a crystal structure according to the invention gives clues regarding their suitability to be used as ligands.

A non-automated procedure to identify suitable compounds with ligand character can be described as follows. As a starting point for the identification, a frame compound is manually inserted into the space, which needs to be filled by the compound to be identified, inside or at the surface of the crystal structure, e.g. in the binding area of a TPR domain in the crystallised polypeptide. Fragments are searched for within the space remaining after the insertion of the frame structure, which can interact with the surrounding crystal structure, and which can be deposited/accumulated on the frame structure. Thus, the search for suitable fragments is carried out according to the geometrical and/or physico-chemical conditions of the three-dimensional structure. The search for suitable fragments can, for example, be carried out as an automated computer search if relevant boundary conditions are given. Possible fragments determined by the operator and/or the computer search are graphically deposited on the original frame structure of the original model, and after every such step, the interaction potential with the target structure area in the crystal structure is calculated. This procedure is carried out until the interaction potential between the compound to be identified and the target structure are is optimised.

The procedure method of steps (c), (d) and (e) can cyclically be repeated until a compound or a compound class is optimised regarding its binding behaviour, calculated according to an interaction potential which forms the algorithm [that] the respective computer program is based upon. The large number of compounds potentially capable of binding, obtained by means of relatively rough characterisation of the binding area of the crystal structure, can increasingly be reduced by adding more detailed prerequisites of physical-chemical or steric characteristics regarding the desired target compound.

Here, an appropriate combination of non-automated and automated search methods for suitable compounds is especially useful. Thus, for example, a compound, which was first identified by an automated computer search in computer databases, could be improved by applying a non-automated method, depositing fragments having suitable, functional groups.

Finally, within the scope of the present invention, it is preferred to synthesise compounds, which were obtained by such automated computer search procedures according to the invention, or, if they were already synthesised and available, to take them from a chemical library and examine them in a suitable biological testing system in procedure step (d) regarding their biological effectiveness. Then, depending on the result of the biological testing system, which can consist of, e.g., a ligand binding assay, further chemical modifications of the previously determined compound or compound classes can be carried out. Here, the application of program packages for the identification of suitable fragments, which could be exchanged with existing fragments of the previously identified compound, or which could be deposited onto that compound, might prove to be appropriate.

An additional subject matter of the present invention are also procedures for the identification of a compound with the property to act as a ligand, typically as an inhibitor of the interaction between a TPR domain and a chaperone protein, where during procedure step (a) in such a procedure according to the invention, a biological testing system is placed at the start. By means of this testing system, a screening for suitable target compounds is carried out. In this case as well, a binding assay can be used as a biological testing system. In the subsequent procedure steps, such compounds (e.g. from a library of chemical compounds) are first identified according to (b), which have shown a positive result in the biological test. These compounds, e.g. inhibiting or possibly also activating, are characterised regarding their, e.g., geometrical and/or chemical properties, especially regarding their three-dimensional structure (procedure step (c)). If the three-dimensional structure of the compounds determined as hits during the biological test is not known a priori, it can be determined by structure determination methods, i.e. X-ray crystallography and/or NMR spectroscopy, or also by modeling, or e.g. semi-quantum-chemical calculations. Then, according to (e), the compounds obtained within the scope of procedure steps (b) and (c) are inserted into the atom structure coordinates of a crystal structure, which are represented according to procedure step (d) as a three-dimensional structure. These can consist of compounds which bind to a section relevant for the physiological binding location, or which bind to the surface of the polypeptide in the crystal structure. Inserting the compound into the crystal structure can be carried out manually depending on the experience of the operator, or it can be carried out in an automated manner, where using relevant computer programs (“Dock” Kuntz et al., 1982, J. Mol. Biol. 161, 269-288, Sybyl/Base “FLEX-X, see reference mentioned above) helps determine a positioning of the ligand with the strongest possible interaction between ligand and the target structure area (procedure step (e)).

By graphically representing a compound obtained in such a manner in combination with the structure present in the crystal structure, additional procedure steps can be carried out which can improve the effectiveness of the target compound. In particular, such a compound previously identified as suitable can serve as basis, or template, for even more effective compounds, e.g. compounds with an even higher binding constant. In this context, procedures and approaches already described in claims 41 to 46 can be applied. A method is preferred which is cyclical to the extent that a structural representation is carried out after the screening in the biological testing system, and that with the help of computer methods more effective compounds are determined on the basis of results obtained in the biological testing systems. These compounds eventually serve as starting point for the next cycle, which is started with a biological testing system. Biological testing systems (in vitro or in vivo) can make statements regarding the quality of the compound, e.g. as an inhibitor of the biological reaction, i.e. the binding event itself, or regarding the binding constant, the toxicity, or the metabolising properties or, should the case arise, regarding the membrane penetration capability of the compound, etc.

Finally, within the scope of the present invention, all such compounds are claimed, which are available, or obtained as a result of a procedure according to one of the claims in 41 to 48.

An additional subject matter of the present invention are procedures for the production of a 3D or crystal structure having at least one polypeptide according to the claims 1 to 22, where in a procedure step (a) the polypeptide is first applied as a coating, synthesised or isolated in an extrusion system, where (b) the polypeptide obtained according to (a) is dissolved in a suitable buffer system, and where (c) the crystallisation is induced by a vapour diffusion method, for example. Typically, according to procedure step (b), a concentrated or highly concentrated solution of the polypeptide/s is present. If crystallisation of at least one polypeptide occurs to form crystals according to the invention, showing crystal structures according to the invention, and if crystallisation is intended to subsequently use the crystals for X-ray structure analysis, then, after crystallisation, X-ray diffraction data are gathered, unit cell constants and symmetry are determined, and the electron density maps are calculated, into which the polypeptide/s are modelled.

An additional subject matter of the present invention are procedures for three-dimensional representation of a crystal structure of unknown structure with at least one polypeptide, containing at least one TPR structure motif, involved in the binding to a chaperone protein, preferably a Hsp70 and/or Hsp90 protein, or preferably at least one TPR structure domain, especially consisting of a Hop protein. Such a procedure is characterised in that the crystal structure having an unknown structure is determined on the basis of a crystal structure, according to the invention, having a known structure, e.g. on the basis of the structure coordinates listed in FIGS. 3C, 3D, or 3E. Here, there are several possibilities to use known structure coordinates of crystal structures according to the invention to determine the structure of polypeptides or polypeptide complexes with so far unknown 3D structures (target structure), which, however, having the known crystal structure according to the invention, show certain homologies in the primary sequence.

In this context, it is possible to use phase information, which can be gathered from known original structure coordinates, e.g. the structure coordinates or parts of these structure coordinates according to FIGS. 3C, 3D, or 3E. To do so, the phase information, which is present or which can be calculated, if the known 3D structure of a crystal structure according to the invention is available, is used to solve an unknown structure, which preferably only distinguishes itself with regards to the known structure in only non-essential conformational deviations (examples that can be mentioned are target structures, to which a ligand or a different ligand as in the original structure is bound, or derivatives, e.g. target structures, being mutants of the original structure). To do so, phase information of the entire structure or part of the known structure, having the unknown structure regarding the crystal structure, is combined with the gathered intensities of the reflexes, and from this combination, an electron density map is calculated for the unknown structure regarding the crystal structure. This method is called “molecular replacement”. Preferably, the molecular replacement is carried out using program package X-PLORE (Brünger, Nature 355, 472-475, 1992).

Another way to use the present crystal structures according to the invention for structure determination of structurally related sequences, or for comparing primary structures with at least partially homologue polypeptide chains, is, according to the invention, that (a) the primary sequence of a polypeptide of unknown 3D structure is compared with a primary sequence of a polypeptide, which shows at least a TPR structure motif or a TPR domain, preferably of a Hop protein, and which within the scope of this comparison, homologous sections are identified between the unknown structure of the polypeptide and the primary sequence of a polypeptide having at least one TPR structure motif, preferably a protein with a TPR binding domain, suitable for the binding to a chaperone protein, particularly preferred a Hop protein, whose 3D or preferably crystal form is known, that (b) the homologous sections are modelled with reference to the known 3D structure, and that eventually according to procedure step (c), with the help of suitable computer programs, the modelled 3D structure of the polypeptide is optimised with regards to its steric ratio conditions.

The so-called alignment of the primary sequences of polypeptides, which need to be compared, of unknown or known 3D structure according to (a), represents the central task for homology modelling. Here, the aligned, corresponding amino acids are assigned to different categories, i.e. positions with identical, similar, vaguely similar or dissimilar amino acids. If the need arises, insertions or deletions between the primary sequences, which require comparison, need to be taken into special consideration when carrying out the alignment. The optimisation of the target structure modelled on the basis of the known 3D structure, carried out according to procedure step (c), can be realised by methods of molecular dynamics simulation, or by energy minimisation (e.g. Sybyl Base by Tripos, see reference above).

In particular, a procedure according to the invention for the determination of crystal structures of unknown structure is preferred, if the crystal form of known structure consists of a Hop protein, or of a protein related to Hop, especially of a protein or of protein sections listed in FIG. 3, and if the crystal structure of a Hop iso protein, or of another related protein, is intended to be determined e.g. by “molecular replacement”, or by “homology modelling”. Also, on the basis of a known Hop crystal structure, according to the invention, of a host organism, a crystal structure can be determined for a Hop complex, possibly with a ligand, by another host organism. Thus, by means of structure modelling, structure coordinates of crystal structures according to the invention can serve as structure models for sequentially homologous polypeptides of unknown 3D structures. Within the scope of homology modelling, program packages are used, especially the modelling package Insight II (Molecular Simulations Inc.) can be used to carry out such modelling. The description of program package Insight II is entirely included in the present disclosure, especially with regards to homology modelling.

An additional subject matter of the present invention are procedures for the identification of compounds, which block the interaction between a polypeptide containing at least one amino acid sequence of a TPR structure motif, preferably of a structure motif of a Hop protein, or of a derivative of such an amino acid sequence and a chaperone protein, where to do so (a) the unknown 3D structure of such a type of polypeptide, corresponding with a polypeptide in a 3D structure according to the invention, is determined according to a procedure described in claims 50 to 52, and where then (b), with the help of a procedure described in connection with the claims 41 to 48, a compound is determined, having the ability to act as an inhibitor of the interaction of a chaperone protein with a polypeptide of first unknown and then a 3D structure determined according to (a). Typically, this will consist of compounds having the TPR domain protein, preferably in the binding area relevant for the binding with the physiological binding partner, thus blocking the interaction due to its complementarity to the TPR binding area, or alternatively, this may consist of compounds, which simulate the binding area of the TPR domain, thus being complementary to the binding location of the chaperone protein.

Finally, within the framework of the present invention, the use of inhibitors and/or, if the case arises, activators of physiological activity of Hop proteins, or of a protein listed in FIG. 3, especially of human Hop protein, is disclosed as well, according to one of the claims 28 to 37, or obtained or available from a procedure according to one of the claims 41 to 48 regarding the manufacturing of a medication, regarding the use as a medication or as a substance, which is contained in a pharmaceutical composition. In a pharmaceutical composition, an inhibitor according to the invention or, if the case arises, an activator can be combined with at least one other substance, and/or the pharmaceutical combination or the inhibitor as a substance is embedded as medication in a formula known to those skilled in the art. Here, the formula will depend especially on the way in which the medication is administered. Administration can be oral, rectal, intranasal or parenteral, especially subcutaneous, intravenous, or intramuscular. Pharmaceutical compositions containing such an inhibitor and/or activator can be in form of a powder, suspension, solution, spray, emulsion or a creme.

An inhibitor according to the invention, or, if the case arises, an activator, can be combined with a pharmaceutically acceptable carrier material having neutral character (such as aqueous or non-aqueous solutions, stabilisers, emulsifiers, detergents and/or additives and, if the case arises, additional colour or taste substances. The concentration of an inhibitor and/or activator, according to the invention, in a pharmaceutical composition can vary between 0.1% and 100%, depending especially on the type of administration. A pharmaceutical composition or a medication containing an inhibitor or, if the case arises, activator, according to the invention, of the interactions between proteins having TPR domains and chaperone proteins, especially Hsp70 or Hsp90, can especially serve as a treatment of tumour diseases, auto-immune diseases, immune suppression, treatment of inflammatory diseases, GVHD or treatment of virus infections.

Additional subjects matters according to the invention are DNA sequences, which code for one of the proteins TTC1, TTC2, TTC3, TTC4, IRSP, SGT, or KIA0719, or DNA sequences, which code for the partial sequences, represented in FIG. 3, of the above mentioned proteins. Here, in this context, reference is made as well to FIG. 3 and to the description of these figures. Especially the additional proteins having TPR domains, listed in FIG. 3, especially the sequences/partial sequences of proteins TTC1, TTC2, TTC3, TTC4, IRSP, SGT and KIAA0719 are disclosed for the first time in the present invention as protein having TPR domains, also having the ability to bind to chaperone proteins, especially to Hsp70 and/or Hsp90. Furthermore, the amino acid sequences of the above mentioned seven proteins, especially the partial sequences according to FIG. 3, are disclosed as sequences with typical co-chaperone function. As an additional subject matter of the invention, this results in the use of these DNA or amino acid sequences for the manufacturing of a medication, especially for the treatment of malfunctions in connection with malformed folding of proteins, cell-pathological conditions of the chaperone system, e.g. hyper- or hypo-functions of the chaperone system. Especially partial sequences, in particular the TPR domains of the proteins, or sections, or fragments, mentioned before, of such TPR domains, can be used as inhibitors of the physiological binding functions of these proteins, which are acting as co-chaperones in this respect.

Attachments A1 to A2 are part of the disclosure of the present patent application.

The present invention is also explained in more detail by the following figures.

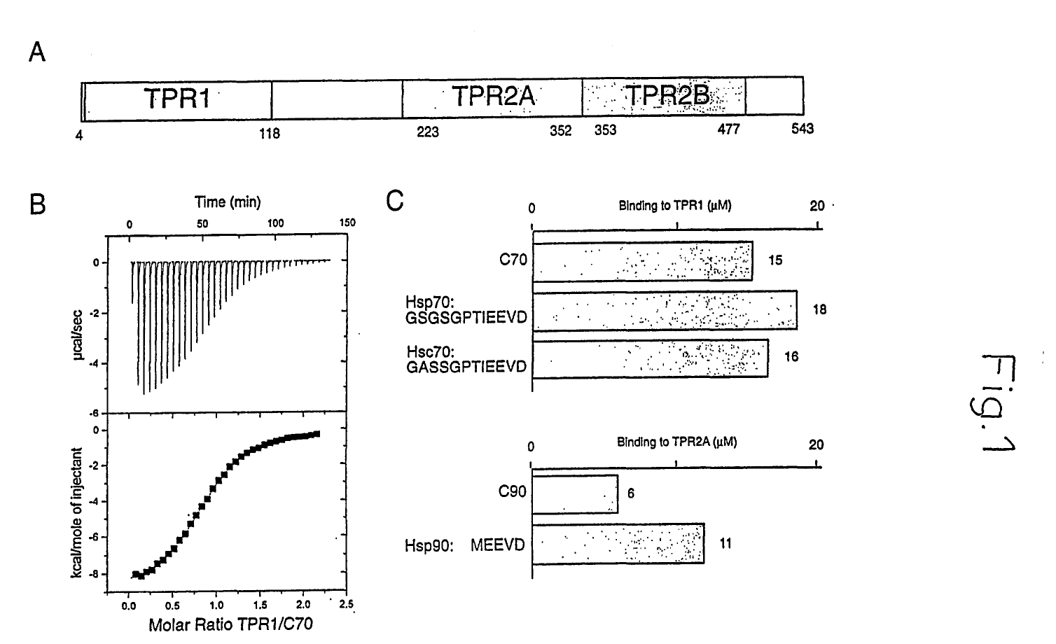

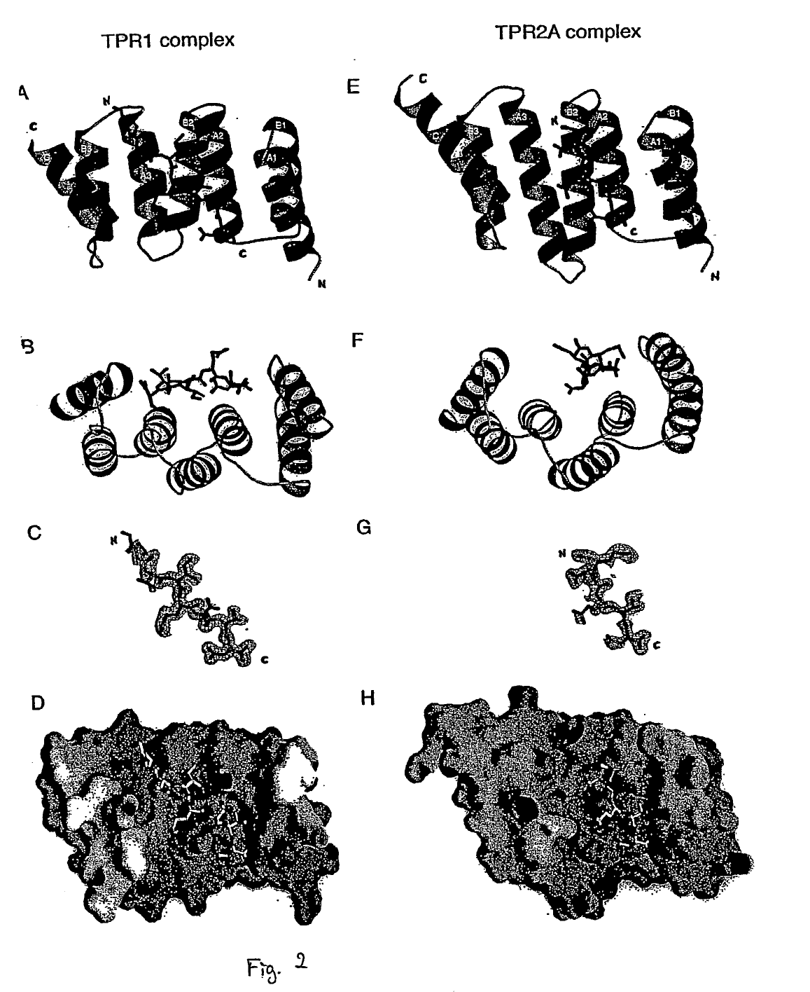

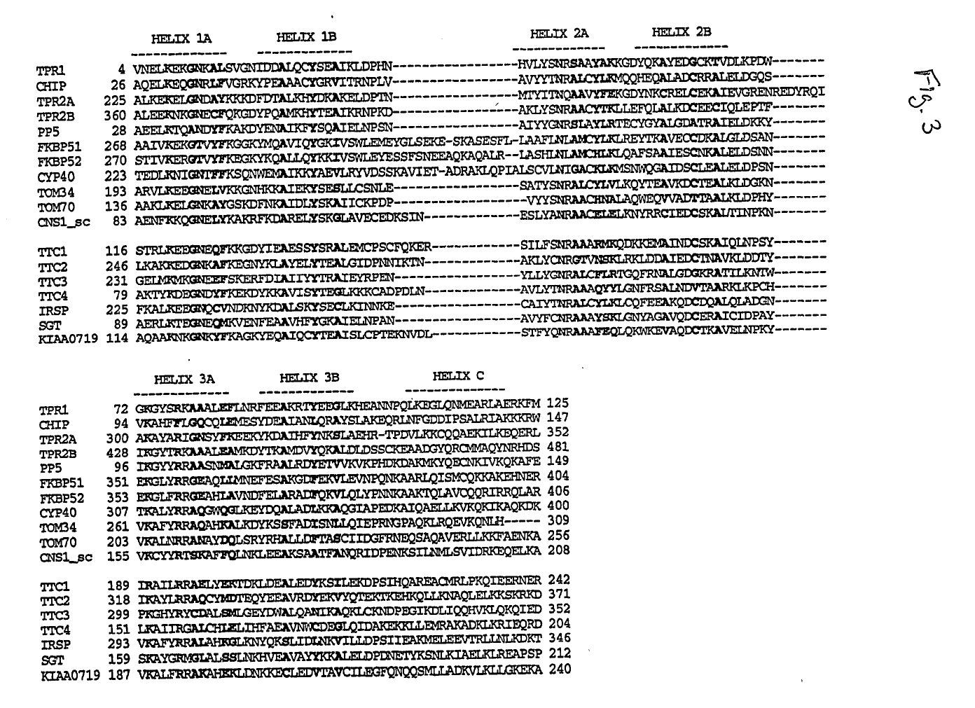

FIG. 1 comprises FIGS. 1A, 1B, and 1C. Here, FIG. 1A shows the sequential rough structure of a Hop protein, taking the three TPR domains present in the Hop protein into consideration, i.e. TPR1, TPR2A, and TPR2B. FIG. 1 also shows the domain borders. Domain TPR1 extends per definition to amino acid 118, domain TPR2A lies between amino acid 223 and amino acid 352, and TPR domain TPR2B lies between amino acid 353 and 477.

FIG. 1B shows the plotting of the calorimetric iso-thermic titration measurements for the determination of the interaction between the TPR1 domain of the Hop protein with the C-terminal 25 kDA domain of Hsp70 (C7). In the top part of the figure, the release of heat after titration of C70 (180•M) with TPR1 (3 mM) at 25° C. is depicted in an incremental manner. In the bottom part, the calorimetric results after integration, normalised with respect to the amount of injected TPR1, are shown. The curve was adjusted to a binding model of stoichiometry 1:1 between the binding partners. The following values were determined: K_{D}=15•μM, stoichiometry N=0.87.

In FIG. 1C, the thermodynamic binding constants (K_{D}) at the interaction of TPR1 with C70 and such peptides, which comprise the last 12 amino acids of Hsp70 and Hsc70 (in the figure at the top), are depicted, as well as those for the binding of TPR2A to C90, and to a Hsp90 pentapeptide (in the figure at the bottom). The titrations were carried out at 25° C. by injecting proteins or peptides in a solution containing TPR1. Typically, the stoichiometric constants (N) were between 0.9 and 1.1. The ITC measurements proved to be optimally reproducible, with standard deviations of approximately 5 to 10% after three measurements had been carried out.

In FIG. 2, figures A to H are shown, where figures A, B, E, and F are models of so-called ribbon figures. The backbone of the TPR domain is depicted in cyan (TPR1) or in magenta (TPR2A), where in FIG. 2A as well as in FIG. 2E, the bound peptides are shown as rod-like models depicted in pink. The N- and C-terminals of the TPR domains or the bound peptides, are marked with the corresponding letters N and C. Helices A and B, present in each TPR structure motif, of the three consecutive TPR structure motifs, are also shown, as well as the flanking helix C. Here, FIGS. 2A, and 2E show the sickle-shaped TPR1 and TPR2 domains.

FIGS. 2B and 2F have the same subject as FIGS. 2A and 2E, where in FIGS. 2B and 2F the complexes consisting of TPR1, having a bound peptide, and TPR2A, having a bound peptide, are rotated through 90° each. Now, the anchor of the peptide, consisting of the two carboxylate groups, is turned to face the viewer. FIGS. 2C and 2G represent (2Fo-Fc-)-electron density maps, which were calculated for the peptide area by using the final model, where the peptide was left out of. FIGS. 2D and 2H are models of the electrostatic potential of TPR1 and TPR2A, which where modelled onto the accessible molecular surface to the extent that it could be calculated and visually represented with the aid of the GRASP program. Corresponding, more detailed explanations can be found in Nicholls et al. (Biographical Journal 64, A166). The negatively charged areas are marked red, the positively charged areas are marked blue, and the neutral areas are marked grey. The bound peptides are also represented, i.e. in a rod-like manner.

In FIG. 3, a sequence-specific comparison (“alignment”) of the TPR domains of such proteins is shown, which either bind to Hsp70 or Hsp90. Conserved amino acid residues involved in electrostatic interactions with the EEVD motif of the ligand, are marked blue, while amino acid residues being in hydrophobe or in van-der-Waals-interaction with the ligand, are marked red. Those amino acid residues shown in bold, are components of the TPR consensus sequence, and are conserved due to the packing of different •-helices of neighbouring TPR structure motifs. All sequences were retrieved from the GenBank database and are of human origin, apart from CNS1 and TOM70, which originate from S. cerevisiae or N. crassa. TPR1 and CHIP consist of Hsp70 binding TPR domains. Hsp90 binding TPR domains are TPR2A, PP5, FKBP51, FKBP52, CYP40, TOM34, TOM70 and CNS1. A ligand for the TPR domain TPR2B of the Hop protein has so far not been identified.

The other sequences TTC1 and TTC2 (they interact with neurofibromin), TTC3 (located in a genome region involved in the pathology of down syndrome), TTC4 (located in an area regularly deleted in sporadic breast cancer), IRSP (a protein connected with infertility symptoms) and SGT (small TPR protein rich in glutamine, which interacts with the non-structural NS1 protein of the parvo virus H-1) also have sequences with TPR domain properties, which can bind to Hsp70 and/or Hsp90. The latter applies also to the human protein KIAA0719 whose physiological function is not known.

FIG. 3A lists the amino acid sequences of an exemplary selection of TPR structure motifs, which can occur in polypeptides. Claims 1 to 22 are targeted to the 3D structure of these polypeptides. The amino acid sequences represented in FIG. 3A, occur in human Hop proteins. Sequences (1), (4), and (7) are components of the TPR domain TPR1, sequences (2), (5), and (8) are components of TPR domain TPR2A, and sequences (3), (6), and (9) are components of TPR domain TPR2B, each of the Hop protein. The 3D structures of sequences (1), (4), and (7) can be gathered from FIG. 3C, representing the structure coordinates of TPR domain TPR1 without the formation of a complex with a ligand. In FIG. 3C or 3D, the TPR structure motifs (1), (4), and (7) can be assigned by means of their sequence (see also FIG. 3) (structure coordinates listed for atoms of N-terminal to C-terminal (Arg118), for which the electron density can be observed). Analogously, the same method can be applied for the 3D structures of TPR structure motifs (2), (5) and (8), which can be gathered from FIG. 3E.

FIG. 3B represents the amino acid sequences of the three TPR domains TPR1, TPR2A and TPR2B, which occur in Hop proteins. Their 3D structure can also be gathered from FIGS. 3C, 3D, and 3E by means of the atom structure coordinates.

FIG. 3C represents the structure coordinates of TPR domain TPR1 (without the formation of a complex with a ligand), as far as electron density could be observed for the amino acids of the sequence. The list of atom structure coordinates contains the structure coordinates of the sequences (1), (4), and (7) according to FIG. 3A. Details regarding structure analysis are contained on pages 1 and 2 (of 39) of FIG. 3C.

FIG. 3D lists the coordinates for the atoms of TPR1 domain of Hop in the complex with the ligand GASSGPTIEEVD. Physical details regarding the implementation of the X-ray structure analysis as well as regarding the crystallised polypeptides are represented on pages 1 to 6 of FIG. 3D.

FIG. 3E represents the coordinates of TPR domain TPR2A of the Hop protein in the complex with the ligand MEEVD. The TPR structure motifs listed in FIG. 3A (sequences (2), (5), and (8) are also contained in their 3D structure in FIG. 3E). Pages 1 to 6 of FIG. 3E present information regarding the crystallised polypeptide and regarding the implementation of the X-ray structure analysis. For FIGS. 3C, 3D, and 3E it is necessary to say that the structure of the terminal, especially the C-terminal amino acids of TPR domains, can not be determined due to the typical flexibility of these end location amino acids.

FIG. 4 is a schematic model of the interaction which can exist between a TPR domain and a complementary peptide. FIG. 4A represents the TPR1/peptide complex, whereas FIG. 4B shows the TPR2A/peptide complex. The colours for the amino acid residue of TPR1, TPR2A and the respective peptides correspond with the colour marking shown in FIG. 2.

FIG. 5 shows the two TPR/peptide complexes superimposed. The superimposing of the so-called double carboxylate bracket of the two TPR1/ and TPR2A/peptide complexes leads to a structural congruency of the two side chains involved in the peptide contacts, and of parts of the bound peptides. The TPR complex is depicted in magenta, the TPR2A complex is depicted in cyan. The amino acid residues of the double carboxylate bracket and the N- and C-terminals of the peptides are marked.

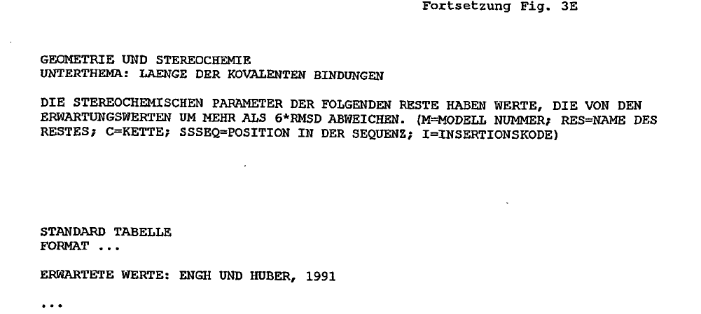

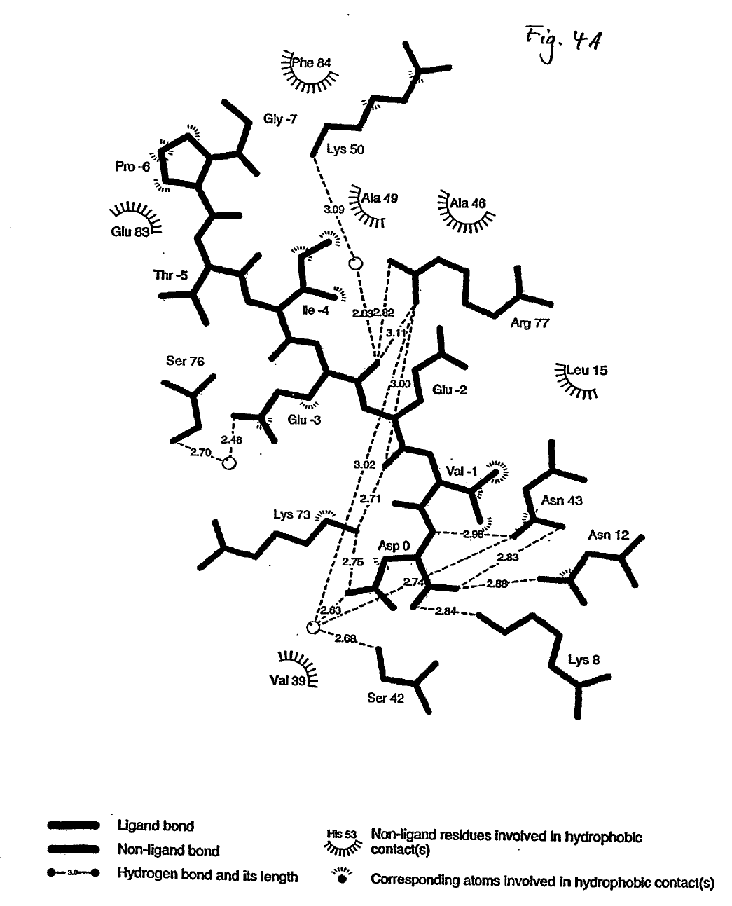

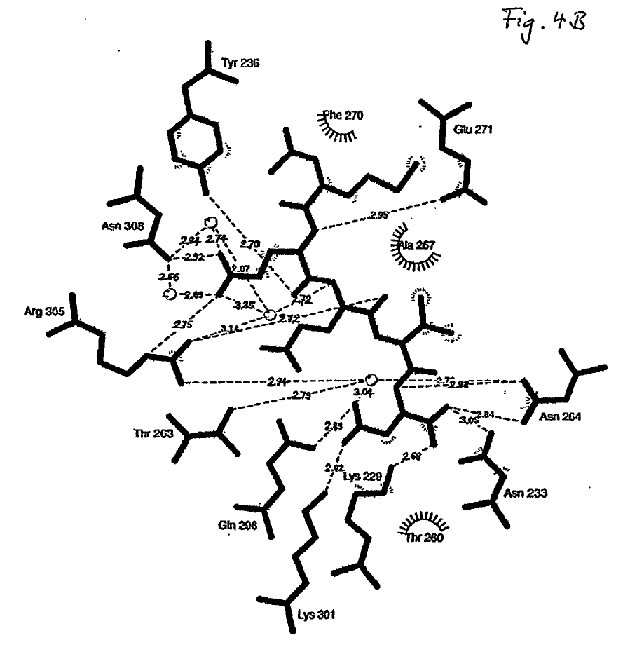

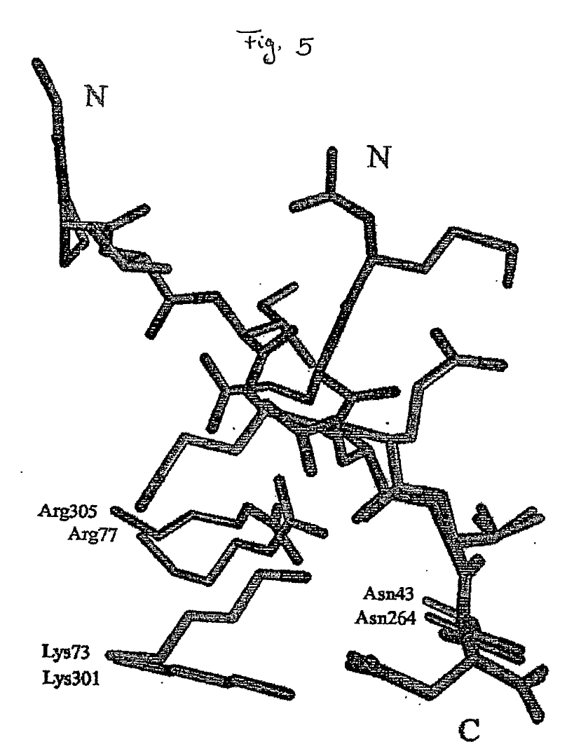

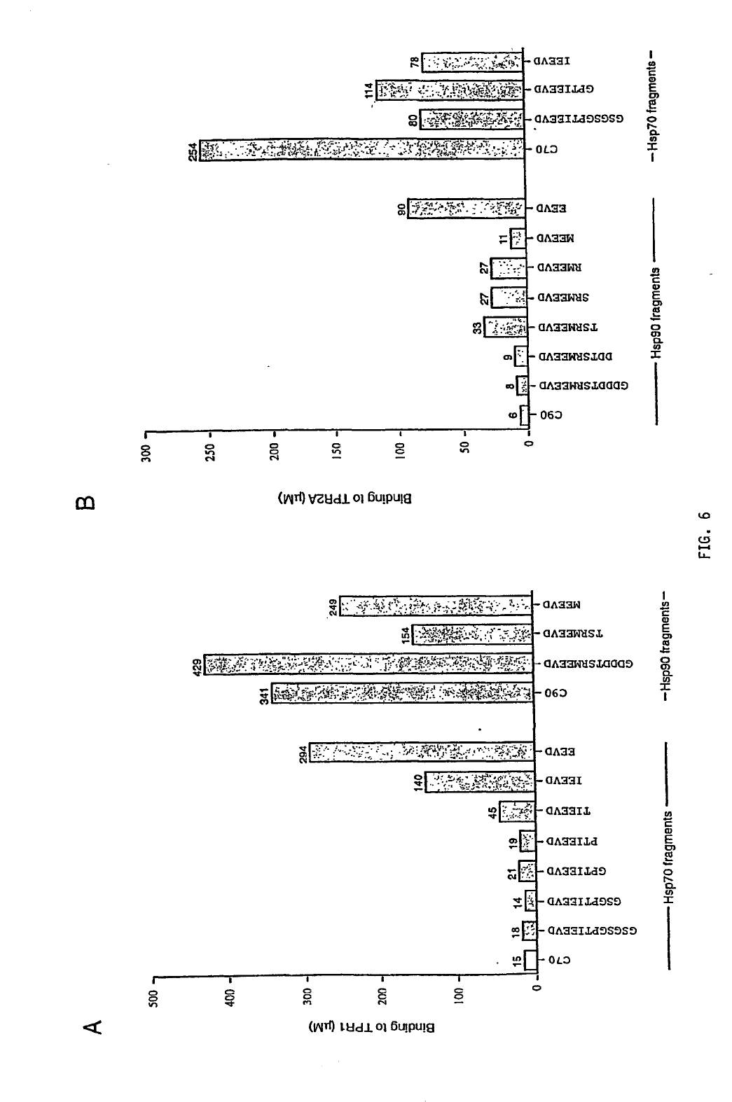

FIG. 6 is a plotting of the thermo-dynamic analysis of the interaction of TPR1 and TPR2A with the C-terminal sequences of Hsp70 and Hsp90. FIG. 6A represents the binding constants (K_{D}) for the interaction of TPR1 with EEVD peptides, which were determined by means of ITC measurements (see FIG. 1). FIG. 6B deals with the binding of TPR2A to EEVD peptides. The sequence GSGGPTIEEVD corresponds with the 12 C-terminal amino acid residues of Hsc70. GDDDTSRMEEVD corresponds with the 12 C-terminal amino acid residues of Hsp90. The-titrations were carried out at 25° C. by injecting peptides, which were dissolved in buffer G at a concentration of 7.5 mM to 15 mM, into a solution, which contained the TPR1 domain (450•μM-950•μM) in buffer G. Typically, the stoichiometry constants (N) lay between 0.9 and 1.1.

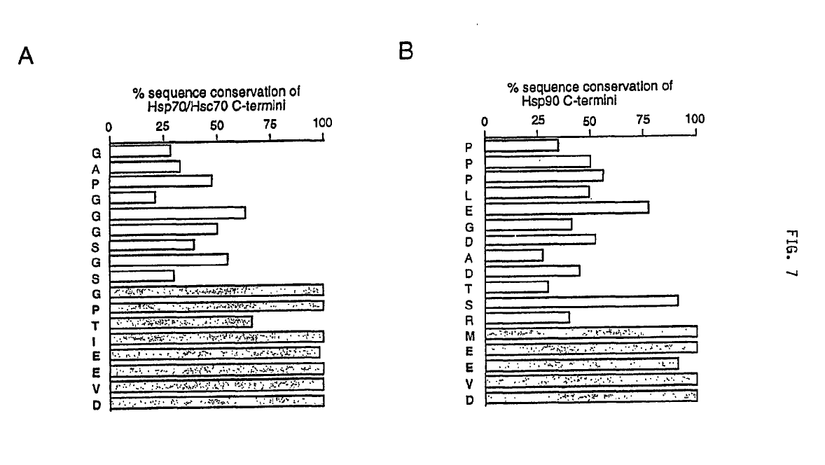

FIG. 7 represents the sequence conservation of the C-terminals of Hsp70/Hsc70 and Hsp90 chaperones. FIG. 7A shows the amino acid conservation of the cytosolic variants of eukaryotic Hsp70 and Hsc70 protein, where 83 sequences were analysed. The conservation of the most frequently occurring amino acid at the respective position is quoted in %. Those amino acids which belong to the structurally arranged peptide area, are marked bold. In FIG. 7B, the amino acid conservation of the cytosolic variants of the eukaryotic Hsp90 protein, is presented in an analogous manner, where 138 sequences where analysed. The last five amino acids, relevant for the TPR binding, are marked bold.

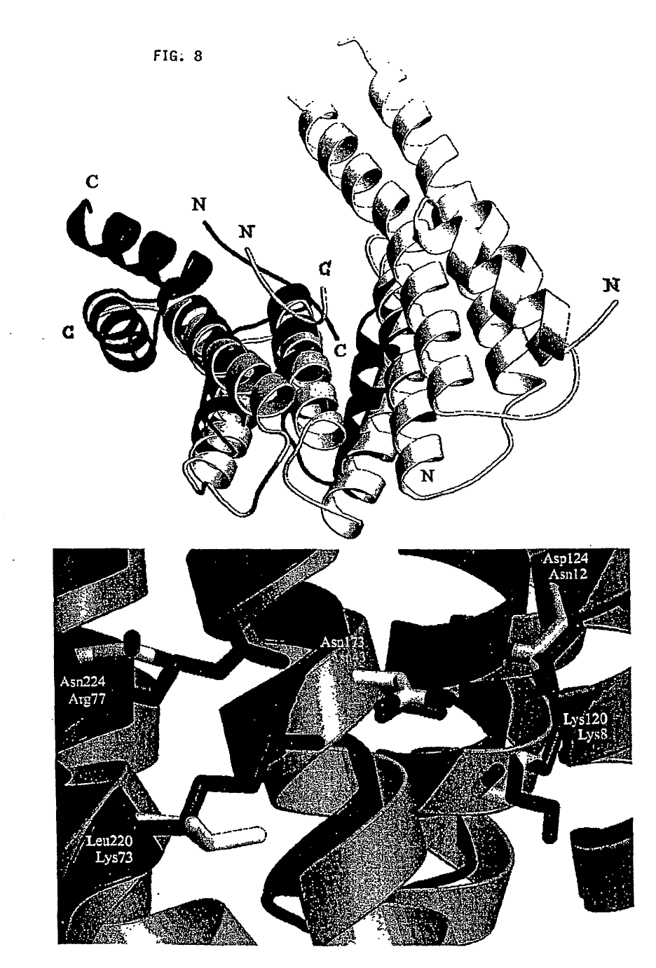

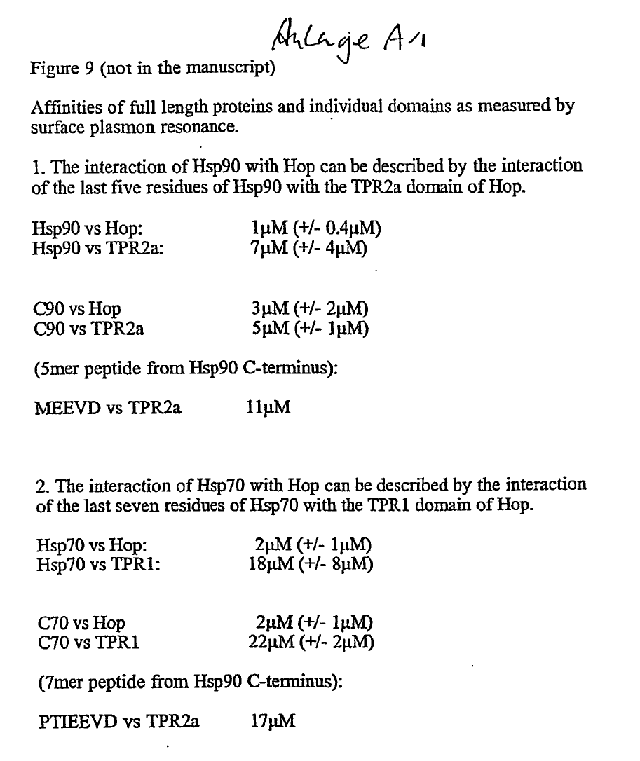

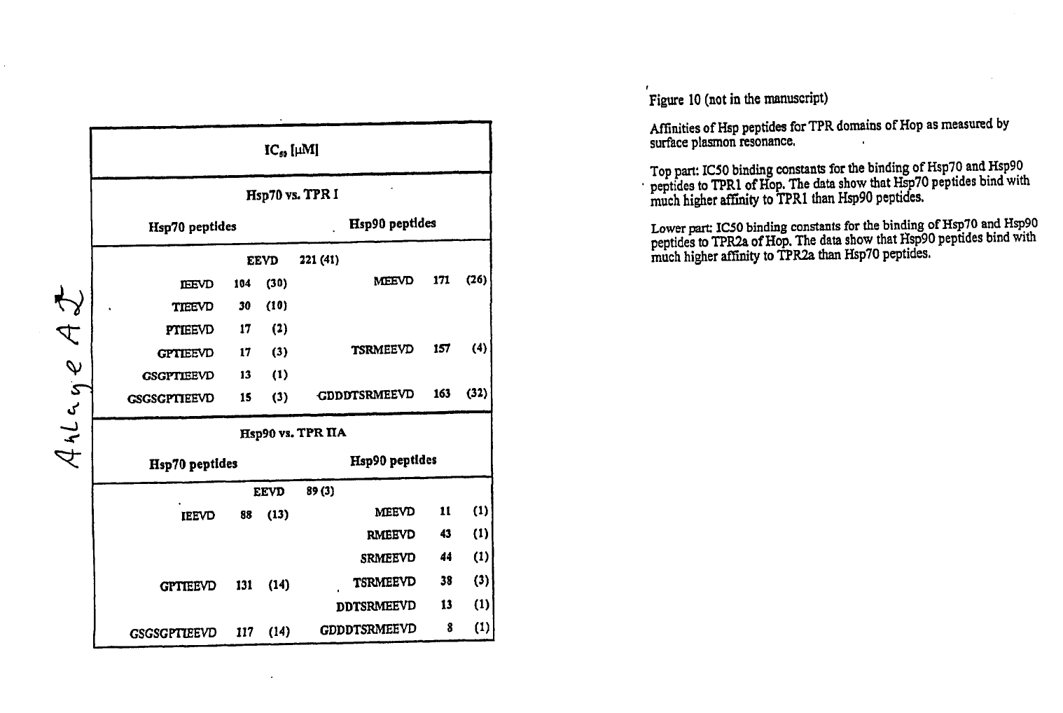

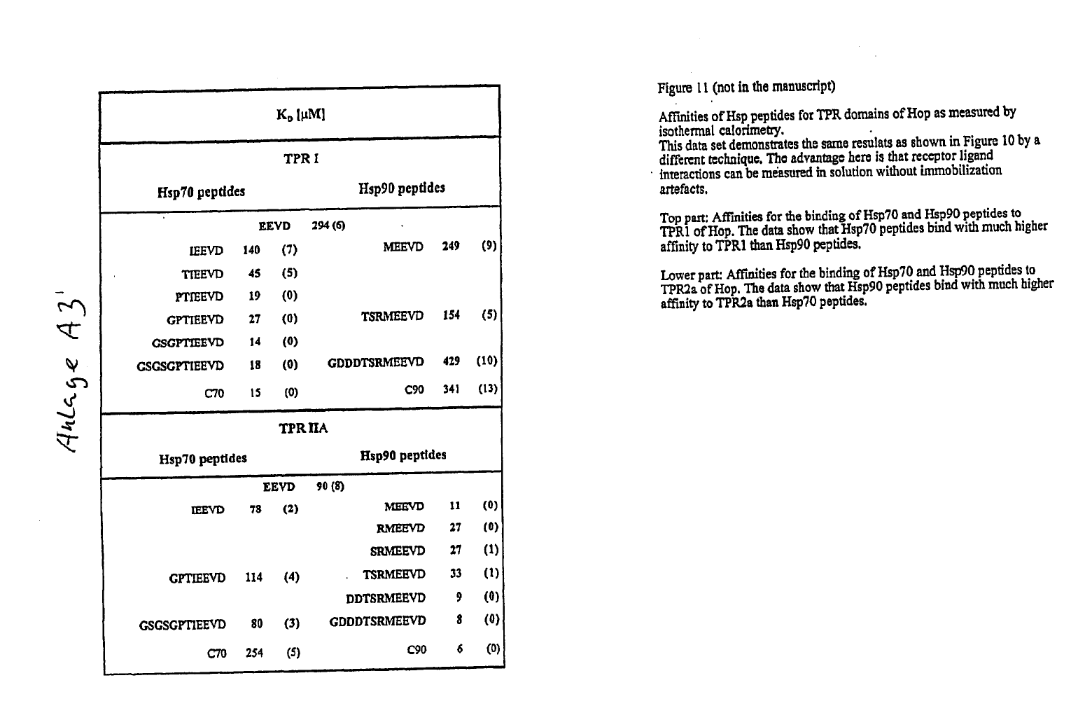

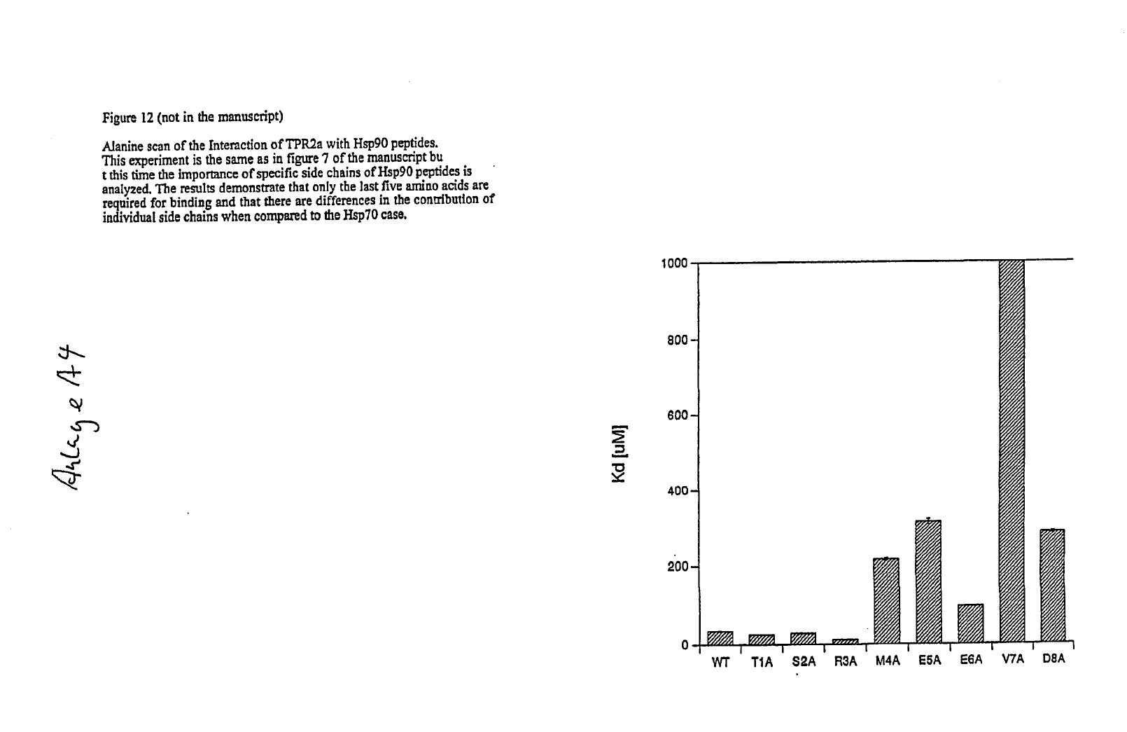

FIG. 8 shows the superimposing of the TPR1/peptide complex with the 14-3-3 peptide complex. FIG. 8A is a representation of the alignment of the corresponding •-helices and the bound peptides. The TPR complex is depicted in magenta, and the 14-3-3 complex is depicted in grey. N- and C-terminals of the domains and the bound peptides are marked. FIG. 8B represents an overlapping of the so-called double carboxylate bracket of TPR1 with the corresponding amino acid of the 14-3-3 domain.

The present invention is explained in more detail by the following embodiments.

Here, the TPR domains TPR1 and TPR2A of the Hop protein (primary sequences marked (1) and (2) listed in FIG. 3B) were crystallised without ligands (FIG. 3C), or in the complex with polypeptide ligands, and subsequently highly resolved crystal structures of the three crystals were obtained by X-ray crystallography.

1. Protein and Peptide Preparation

Codons 1-118 (TPR1) and 223-352 (TPR2A) of human Hop protein were cloned in the proper reading screen into the EheI interface of the plasmide pPROEx Hta (by the company Life Sciences) for expression in E.coli BL21 (DE3) as fusion proteins with detachable N-terminal hexahistidine tags. The soluble proteins were purified by means of chromatographic procedures on Ni-NTA (by the company Quiagen) and Superdex 200 (by the company Pharmacia). The his tags were detached using TEV proteasis, where an additional glycerine residue remained in front of the N-terminal. The proteins were concentrated by means of ultra-filtration to obtain a concentration of 40 mg/ml. In order to determine the structure of the domain TPR2A, a form marked with selenomethionin was produced by means of expression in the E.coli strain B834 (DE3). A peptide marked with selenomethionin (the five C-terminal amino acids of Hsp90 with the sequence MEEVD (single-letter-code)) was used for the co-crystallisation. Integration of the methionines was confirmed by mass spectrometry. The C-terminal fragment of human Hsp70 (C70, codons 382-641) was cloned, expressed and purified according to the same method. The C-terminal domain of the human Hsp90 (C90, codons 625-732) was, as described in Young et al. (1998, J. Biol. Chem. 273, 18007-18010), expressed and purified.

For the synthesis of Ac- (Se)Met-Glu-Glu-Val-Asp-OH, the L-selenomethionin was transformed into Fmoc- (Se)Met-OH by reaction with Fmoc-OSu under standard conditions (crystallisation from ethylacetate/hexane). The tetrapeptide H-Glu(OtBu)-Glu(OtBu)-Val-Asp(OtBu)-resin was synthesised on Wang resin (by the company Bachem, Bubendorf) according to standard Fmoc/HBTU/HOBt solid phase protocols. In a double surplus, Fmoc-(Se)Met-OH was coupled twice to the peptide resin with HBTU/HOBt/DIEA (1:1:2). After splitting, induced by piperidin, of the Fmoc protection group, the N-terminal acetylisation was carried out with an eight-fold surplus of Ac20 in the presence of DIEA. The resin splitting step and de-protection step was carried out my means of argon saturated TFA/TIS/H₂O (93.5:5:1.5) at room temperature for a period of 90 minutes in argon atmosphere. The resin was filtered off, and the product was precipitated with methylert-butyl-ether/hexane. The raw peptide was purified by preparative RP-HPLC (elution: linear gradient of 0.1% aqueous TFA and 0.08% TFA in CH₃CN). The product was obtained after lyophilisation. All other projects were synthesised with acetylised N-terminals using solid phase FMOC chemistry.

2. Crystallisation and Data Collection

The crystals of the polypeptide corresponding with the TPR1 domain of Hop (FIG. 3B, sequence (1)), in complex with a peptide (GASSGPTIEEVD, C-terminal dodecamer of Hsp70), were cultivated at 20° C. by means of the “hanging drop” method, using vapour diffusion procedures. Equal volumes (1.5 μ•l) of the protein peptide complex (protein:peptide=1:1.3) were blended with a reservoir solution at a concentration of 20 mg/ml in 15 mM HEPES (pH 7.5), containing 100 mM TRIS (pH 8.5), 24% (w/v) PEG MME 2000, 10 mM NiCl₂ and 15% (w/v) xylitol. Crystallisation was improved using micro seeding, where the crystals reached their full size (typically 150×150×70•μm³) within two weeks. Then, the crystals were shock-cooled in liquid nitrogen and conserved at 100 K. during data collection. For this procedure, an Oxford cryostream device was used. The crystals belonged to the space group P4₁ (a=75.47 Å, c=42.89 Å) with two molecules per asymmetric unit and a solution proportion of 45%. Four nickel atoms and four TRIS molecules were found in the structure. The highly resolved data sets were collected at the ray source X12B of the National Synchrotron Light Source in Brookhaven, using an ADSC quantum-4 CCD detector. More details regarding the data collection can be gathered from table 1.

The crystals of TPR2A (amino acid sequence as quoted in FIG. 3B for the polypeptide marked (2)) were cultivated in complex with a pentamer (MEEVD, corresponding with the five C-terminal amino acids of the chaperone protein Hsp90) as described above. For this process, identical amounts (1.8•l) of the protein peptide complex (protein:peptide=1:1.3) were blended with a reservoir solution at a concentration of 15 mg/ml in 50 mM TRIS (pH 7.5), 2mM DDT, containing 100 mM TRIS (ph 8.5), 20% (w/v) PEG MME 2000, 5 mM NiCl₂ and 10% (w/v) xylitol. After one week, the crystals started to show, and they reached their full size (typically 100×50×50 μm) within three weeks. It was established that the nickel ions and the peptide were crucial for crystallisation. A nickel ion, which was structurally fixed to a high degree, was found, establishing crystal contact. It was used to determine the phase information. The crystals belonged to the space group C2 having one molecule per asymmetric unit and a solution proportion of 40%. The native data set was collected up to a resolution of 1.9 Å at the ray source ID14-3 at the ESRF, using a MarCCD detector. The multiple anisomorph diffraction data (MAD) of marked TRP2A peptide complex was recorded at the MPG/GBF wiggler ray source BW6/DORIS at DESY, using a MarCCD detector. The data sets were collected around the absorption edges of Ni (Ni1:peak, Ni2:inflection) and Se(Se1:peak, Se2:inflection), using one single protein crystal. All data sets were processed using the HKL software program package (Otwinowski and Minor, 1997, Macromolecular Crystallography, Pt A, pages 307-326). More details regarding data collection can be gathered from table 2.

3. Structure Determination and Structure Refinement

The structure of the TPR1 complex was determined by molecular replacement, using the Amore program, which is part of the CCP4 program suite (Bailey, 1994, Acta Crystallographica Section D-Biological Crystallography, 50, 760-763). Parts of the previously determined crystal structure of TPR1L (amino acids 1-140) served as a search model. The TPR1 peptide complex was refined using the program CNS (Brünger et al., 1998, Acta Crystallographica Section D, Biological Crystallography, 54, 905-921) using all diffraction data between 20 and 1.6 Å resolution, where 10% of the data for cross validation (see table 1) were excluded. All refinement steps were carried out without using non-crystallographic symmetry. In both crystallographically independent TPR1 domains, electron density could be observed in the asymmetric unit (chain A and chain B) for the amino acid residues from A2 to A118, and B1 to B115. Due to the flexibility, the residues A2, B1, B2, and B110 were modelled as alanines. The positions of the peptides were determined from a difference electron density map. In both cases, only the last eight amino acids GPTIEEVD of the peptide could be assigned to an electron density. The difference electron density map also allowed identification of the four nickel atoms (two per monomer) and of four TRIS molecules (two per monomer). At histidine 36, two nickel binding locations were located each for the chains A and B. Occupancy was manually set to 0.5 in order to adjust the temperature factors to the temperature of the bound histidines. Two completely occupied positions were located at histidine 101 (for the chains A and B). Each nickel atom which is bound to the histidine, is in addition entangled as a complex with two TRIS molecules.

The final model, containing 1960 protein atoms, 36 hetero atoms and 245 solvent molecules, converged at an R factor R_{work}=18.2% (R_{free}=21.6%). The mean standard deviation for the binding lengths is 0.008 Å, and for the binding angles 1.3°. The previous description of the structure dealt with only one of the two chains in complex with the bound peptide (chain A or chain C for the peptide) of the two chains A and B contained in the asymmetric unit.

To determine the TPR2A structure (table 2), the MAD data sets were placed on an approximate absolute scale and treated as a special case of MIR data, where different programs of the CCP4 program suite (CCP4, 1994) were applied. The position of a nickel atom was determined by analysis of an anomalous difference Patterson map from the data at a wave length of maximum f_{N1}″ (Ni1). These nickel binding location was also used to calculate the MAD phases by means of MLPHARE (CCP4, 1994). These phases were then continued to be used to identify the two selenium binding locations from the anomalous difference Fourier map, which was calculated by means of the data obtained at maximum f_{Se}″ (Se1). In order to finally determine the phase, the wavelength of the inflection point of nickel (Ni2) was used as a native data set, and the other three wavelengths (Ni1, Se1, Se2) were treated as separate derivatives. The phases were calculated in resolution range between 17.0-2.1 Å, and their initial mean figure of merit of 0.74 was improved by the solvent flattening and histogram matching method, using the program DM (CCP4, 1994). The obtained electron density map was of high quality and allowed to assign approximately 90% of the final structure by using wARP (Perrakis et al, 1997). The atom models were reviewed and further improved using the program O (Jones et al., 1991, Acta Crystallographica, Section A 47, 110-119). The refinements were carried out using the program CNS (Brünger et al., 1998, Acta Crystallographica, Section D, Biological Crystallography 54, 905-921) against the native data set using all diffraction data between 10 and 1.9 Å resolution (except 10% of the data for the cross validation). The amino acids Lys223 to Gln349 (TPR2A domain) were assigned to the electron density, just as the amino acids Met-4 to Asp0 of the pentapeptide. Due to their flexibility, the residues A291 to A294 and A348 were modelled as alanines. The nickel binding location used within the scope of the phase determination proved to be a bridge of a crystal contact of His247 to His321 and Lys325 from the symmetry related molecule.

The final model contained 1086 protein atoms (44 for the peptide), 1 hetero atom, and 152 solvent molecules, which converged at an R factor of R_{work}=18.1% (R_{free}=21.9%) for all data without any sigma border value. The standard deviation for the binding length was 0.008 Å, and for the binding angles it was 1.2°. All residues are located in the favoured or at least in the generally allowed areas of the Ramachandran plotting, as calculated by means of the program PROCHECK (Laskowski et al., 1993).

4. Isothermal Titration Calorimetrics (ITC)

The binding of protein fragments and peptides to the TPR domain was measured by means of isothermal titration calorimetrics (Wiseman et al., 1989. Anal. Biochem. 179, 131-137), using a MicroCal MCS titration calorimeter (MicroCal Inc., Northhampton, USA). 40-50 aliquots of 5•μl peptide solution (7.5 mM-15 mM) were titrated at 25° C. by injection into a 1.36 ml TPR1 (TPR2A) solution (450•M-950•M) into the chamber. Alternatively, a solution of 3 mM TPR1 (TPR2A) in 180•M C70 (C90) solution was titrated in the chamber. The peptides were dissolved in and the proteins were dialysed against the buffer G (25 mM, pH 7.5, 100 mM Kac, 5 mM MgAc₂). Typically, the injections were continued past the saturation limits in order to be able to determine the heat of the ligand solution. After subtracting the solution heat, the calorimetric data was analysed, where an assessment software was used, which is provided by the manufacturer (version 2.9; MicroCal Software, Inc.).

Results of the Embodiments

1.

By means of a restricted proteolysis three defined domains of human Hop protein have been identified: TPR1, TPR2A and TPR2B (FIG. 1A). All three domains are TPR domains.

2.

It has been shown with the isothermic, calorimetric measurements that a 25 kDa fragment of human Hsp70 also continues to feature the substrate binding domain and that the authentic Hsp70 C-terminal bound to the domain TPR1 with an affinity of 15•M and a stoichiometry factor (N) near 1 binds to the domain TPR1 (FIGS. 1B and 1C). The same affinities were obtained for a Dodekamer with amino-acid sequences of C-terminals of Hsp70 and Hsc70, which are nearly identical, as ligands (FIG. 1C). A 12 kDa fragment of Hsp90 (C90) which includes the dimerisation domain and the authentic C-terminal of Hsp90 (amino acids 629-732), binds to the domain TPR2A with an affinity of 6•M and a stoichiometry factor near 1 (FIG. 1C). A peptide which only consists of the last five amino acid residues of Hsp70 still continues to bind to the domain TRP2A with an affinity of 12•M (FIG. 1C).

It follows from this that the binding of TPR1 to C70 can be completely described with the interaction of TPR1 with a C-terminal Dodekamer of Hsp70, while the interaction of TPR2A with C90 is essentially facilitated by the five C-terminal amino acids of Hsp90, i.e. a pentapeptide. The measured affinities are otherwise comparable to those which were determined for the interaction of SH3 domains with their peptide ligands (Kuriyan and Cowburn, 1997, Annual Rev. Biophys. Biomol. Struct. 26, 259-288).

3. 3D structures of the complexes of TPR domain and the peptide ligands

The two TPR domains TPR1 and TPR2A of Hop were crystallised as a complex with their respective peptide ligands and their highly dissolved crystal structures were clarified. The result of the examinations was a 3D structure which presented itself as a crystal shape of the two, possibly complexed domains. Both TPR domains establish meander-shaped structures consisting of seven •-helices (FIGS. 2A and 2E) which are arranged in a counter sequence (head to tail) similar to the structure of the peptide-free TPR domain of PP5 (Das et al., 1998, EMBO J. 17, 1192-1199). As opposed to the sequence of TPR1, the TPR domains PP5 and TPR2A feature an insertion between repeat 2 and 3, which leads to an extension of the helices 2B and 3A by one turn (FIG. 2E, FIG. 3) respectively. In both structures helix C, which is placed in a C-terminal direction in relation to the TPR consensus blocks, is an integral component of the TPR domain (FIGS. 2A, 2B, 2E and 2F).

The TPR meander now form sickle-shaped (“cradle”) grooves which house the peptides in an extended conformation (FIGS. 2B, 2F, 2D and 2H). The bound peptides are only in contact with the side chains of the A-helices which form the inner surface of the cradle (FIGS. 2B and 2F). By means of a simulated annealing (2Fo-Fc) electron density map of the TPR1-bound peptide, which was calculated without including the peptide, it has been shown that the last seven peptide amino acid residues in the complex with the domain TPR1 are defined very accurately (FIG. 2C). A similar electron density map which was calculated for the peptide bound to the TPR2A structure, shows that all of the five residues of the bound peptide are well structured (FIG. 2G). The extended conformation of the peptides ensures that the maximum possible surface area in regard to the TPR domains is available as contact area and that the specific recognition of short amino acid pieces with sufficient affinity is made possible in this manner.

In the following description of the TPR peptide complex the C-terminal Asp-residue of the peptides is referred to as Asp0. The preceding residues are numbered in descending order with Val-1, Glu-2, Glu-3, Ile-4, Thr-5, Pro-6 and Gly-7 for the Hsp70 peptide (FIG. 4A) or as Met-4 for the Hsp90 peptide (FIG. 4B). Only the last eight residues in the Hsp70 Dodekamer peptide are arranged in the TRP1 complex in such a manner that they are recognisable in the concluding electron density map. The peptide side chains Pro-6, Ile-4, Val-1 and Asp0 play a part in interactions with the TPR1 domain. The other side chains in the peptide section with the controlled structure, especially both of the nearly absolutely conserved glutamic acid side chains in positions -2 and -3, are exposed with regard to the solvent (FIG. 2C; FIG. 4A). All of the other five peptide residues in the TPR2A complex are clearly visible on the concluding electron density map. With the exception of Glu-2, all side chains of the Hsp90 peptide play a part in the interactions with domain TPR2A (FIG. 4B). All electrostatic contacts between TPR domains and peptides exclusively register for segments of the EEVD motif, while the sections of the bound peptides, positioned in N-terminal direction to the EEVD motif, exclusively enter into hydrophobic and van-der-Waals-interactions (FIGS. 4A and 4B).

Three kinds of hydrogen bridge binding interactions are involved in the development of peptide binding to the domains TPR1 and TPR2A: (a) interaction with the peptide backbone independent of sequence, (b) sequence-specific interaction with the peptide side chains and (c) contacts with the C-terminal main chain carboxylate of the last peptide residue Asp0 (FIGS. 4A and 4B). As further described below, the contacts referred to last and the electro-static interactions with the main chain carboxylate of Asp0 form a so-called “two carboxylate clamp”, which is highly conserved between the two complexes.

A large number of direct hydrogen bridge binding interactions of the TPR domains with the Hsp peptides are directed to the peptide backbone so that no sequence-specific characteristics are utilised for the binding. In the TPR1 complex (FIG. 4A) the side chain carbonyl group of Asn43 (of TPR1) is involved in direct backbone interactions, which are formed with the Asp0 backbone amide of the Hsp70 peptide and the amino side chain of Lys73. In turn, the amino side chain of Lys73 forms a hydrogen bridge binding with the main chain carbonyle group of the peptide residue Glu-2. Arg77 of the TPR1 domain plays a key role in connecting to the backbone of the peptide. Its guanidinium group forms three direct hydrogen bridge bindings with the peptide carbonyl groups, specifically one at the -2 and two at the -3 position. An additional hydrogen bridge binding to the carbonyl group of Glu-3 is placed (FIG. 4A) with a firmly bound hydrogen molecule which is being positioned in TPR1 by Lys50.

In the TPR2A complex the carbonyl side chain of Asn264 makes contact with the Asp0 backbone amide of the Hsp90 peptide, the guanidinium group of Arg305 forms a hydrogen bridge with the main chain carbonyl group of Glu-2, the hydroxyl group of Tyr236 forms a hydrogen bridge with that of Glu-3 and the side chain of Glu27 interacts with the amide group of Glu-3. In addition, contact between the hydroxyl group of Tyr236 with the main chain carbonyl group of Glu-3 can be observed.

The only peptide side chain in the TPR1 complex which is recognised by an electro-static interaction, is the side chain carboxyl group of Asp0, which interacts with Lys73 (FIG. 4A). An additional side chain contact is facilitated by a controlled water molecule which is coordinated tetrahedrally by the side chain carbonyl groups of Asp0 and Asn43, the guanidinium group of Arg77 and the side chain hydroxyl of Ser42. The electro-static interactions of TPR2A with the Hsp90 peptide are similar to those in the TPR1 complex, with the exception of only a few differences.

TPR2A forms two hydrogen bridge bindings with the side chain of Asp0 via Lys 301 on the one hand and with the side chain amide group of Gln298 on the other. Exactly as with the TPR1 complex, a tetrahedrally coordinated water molecule is in contact with the Asp0 side chain, positioned by the side chain carbonyl group of Asn264, the guanidinium group of Arg355 and the side chain amide group of Asn233 (FIG. 3C and FIG. 4). Unlike in the TPR1 complex, the side chain of Glu-3 in the TPR2A complex is part of a compact network of hydrogen bridge bindings with the TPR domain. It is part of direct contacts with the guanidinium group of Arg305 and with the side chain amide group of Asn308, while in addition, it is integrated into a network of indirect interactions, a process involving controlled water molecules (FIG. 4B).

In both TPR complexes the main chain carboxylate of Asp0 is fixed in its position by three additional strong hydrogen bridge bindings with the side chain amines of Lys8 (Lys229), Asn12 (Asn233) and Asn43 (Asn64) of the TPR1 or TPR2A domain. Beyond the previously described electro-static interactions between TPR domains and the EEVD motif of the bound peptides, the peptide residue Val-1 is involved with hydrophobic and van-der-Waals-contacts, which in both complexes are also essentially conserved (FIGS. 4A and 4B).

As described previously, the five amino acid residues of TPR1 which are involved in electro-static interactions with the EEVD motif (Lys8, Asn12, Asn43, Lys73 and Arg77), form a “two carboxylate clamp” which firmly interacts with the terminal Asp residue of the bound peptide. These residues show clear structural commonality with the equivalent residues of TPR2A (Lys229, Asn233, Asn264, Lys301 and Arg305) as soon as their C•atoms are superimposed (FIG. 5). The mean standard deviations (rmsd) for the five positions of the “two carboxylate clamp's” amount to 0.75 Å (compared with 1.75 Å for all of the domains). Excepting Lys73 (Lys301), all side chains are indeed in the same position in both structures. This alignment also leads to the two bound peptides being superimposed. It thereby becomes visible that the positions of Asp0 and Val-1 are surprisingly well arranged on top of the other, while the N-terminal sections of the two peptides from the C- to the N-terminal diverge increasingly, whereby use is made of different areas when entering into interactions with the respective TPR domain. It has to be mentioned that the side chain residues of the domains TPR1 and TPR2A forming the “two carboxylate clamp”, are highly conserved for all TPR domains of which is known that they bind the C-terminal domains of Hsp70/Hsc70 or Hsp90 (FIG. 3). This leads to the assumption that these TPR domains connect to the C-terminal carboxylate of Hsp70 or Hsp 90 via a very similar network of electro-static interactions (FIG. 4).

A protein data library search shows that seven additional TPR proteins exist which should be capable to bind a C-terminal aspartate via the “two carboxylate clamp”, including several human proteins which are involved in disease events (FIG. 3). In particular, the proteins TTC1, TTC2, TTC3, TTC4, IRSP, SGT and KIAA0719 have to be mentioned so that according to the invention the functionality of these afore-mentioned proteins as TPR proteins with co-chaperone function for Hsp70 or Hsp90 are disclosed. Uses of the afore-mentioned proteins, listed in FIG. 3, as co-chaperones of Hsp70 and/or Hsp90 according to the invention are therefore specifically disclosed.

The amino acid residues located further up-stream from the EEVD motif, ensure Hsp70 specificity by hydrophobic interaction. As per the findings according to the invention, the electro-static interactions of the domains TPR1 and TPR2A with the EEVD motif as described above, are not suited to make a distinction between the C-terminals of Hsp90 and Hsp70. It can be seen from the results of the structure determination and the 3D structures of the TPR/peptide complexes according to the invention that there are additional contacts with the peptide residues which are positioned in N-terminal direction to the EEVD motif (FIGS. 4A and 4B). These interactions are decisive for the binding of peptides which has to occur with a physiologically relevant, high affinity (FIG. 6A). While the C-terminal heptamer peptide of Hsc70 binds to the TPR1 domain with the same affinity as the complete C-terminal domain of Hsp70/Hsc70, the separation of the amino acid residues, positioned in an N-terminal direction to the EEVD motif, leads to a distinct reduction of affinity from 15-20•M to approximately 300•M. Furthermore, the peptide IEEVD, which in terms of its length equals the Hsp90 peptide, binds significantly weaker with an affinity of 140•M than the heptamer peptide (FIG. 6A).

The hidden surface area for the structured octamer peptide in the TPR1 complex amounts to 1330 Å² but only to 650 Å² for the four residues of the IEEVD motif. The significant interactions responsible for the 20-fold increase in affinity, are therefore based exclusively on the hydrophobic or van-der-Waals-interaction of TPR1 with the side chains of Ile-4 and Pro-6 of the Hsp70 peptide (FIG. 4A). Each hydrophobic contact of the peptide with TPR1 is essentially limited to a specific A-helix. Pro-6 is placed in a hydrophobic pocket, formed by the Glu83 and Phe84 of helix A3. Ile-4 binds into a pocket which is formed by the amino acid residues Ala46, Ala49 and Lys50 of helix A2 (FIG. 4A). The hydrophobic residue in the EEVD motif, namely Val-1, enters into hydrophobic interactions with Asn12 and Leu15 in helix A1 and with Asn43 in helix A2 (FIG. 4A).

The peptide bound in the TPR2A complex is clearly shorter than that bound in the TPR1 complex. The overall hidden surface area of the TPR2A/MEEVD complex amounts to 930 Å² and 750 Å² for the residue EEVD. Nonetheless, the affinities measured for the Hsp⁹⁰ penta-peptide MEEDV in relation to TPR2A are of the same order of magnitude as the affinities of the Hsp70 peptide GPTIEEVD for TPR1, whereby longer peptides, however, do not bind with a higher affinity (FIG. 6B). The two additional hydrogen bridge bindings which TPR2A enters into with the Glu-3 of the peptide, seem to balance the smaller hydrophobic area for interaction (FIG. 4B). Furthermore, it has to be mentioned that as a result of the stronger interaction between TPR2A and the EEVD motif, in this case the hidden surface area is approximately 100 Å² larger as compared with the hidden surface area as it is calculated in the case of an interaction between TPR1 in a complex with the sequence EEVD (without further N-terminal amino acids). Met-4 of the Hsp90 peptide plays a part in strong hydrophobic interactions with a pocket which essentially is formed by the side chains of Tyr236 and Glu271. Val-1 of the EEVD section binds to a hydrophobic pocket formed by Asn233, Asn264 and Ala267. Consequently, with the stronger interactions in the Hsp90-EEVD section a shortening of the penta-peptide MEEVD relative to EEVD results in a significant, albeit moderate loss of binding affinity (from 11•M to 90•M), while domain TPR1 only binds the EEVD peptide with an affinity of 300•M (FIG. 6A and FIG. 6B).

The sections which are positioned in an N-terminal direction to the EEVD motif are strikingly different from the C-terminals of Hsp70/Hsc70 and the Hsp90 proteins (FIG. 7A and B). This leads to the assumption that these sequences are not only important for the binding behaviour with high affinity but that they are also responsible for the TPR specificity. The eight C-terminal amino acids are nearly completely conserved in all cytosolic variants of eukaryotic Hsp70 and Hsc70 proteins (consensus: GPTIFEEVD). Furthermore, these are also the amino acid residues which in the crystal structure of the TPR1 peptide complex are structurally well defined. It is highly likely that this sequence is solvent-exposed because it is connected with the peptide binding domain of Hsp70/Hsc70 via a flexible linker area of approximately nine amino acids, consisting of a sequence of Ala, Ser and Gly residues (FIG. 7A). The sequence comparison (“alignment”) of C-terminals of cytosolic variants of eukaryotic Hsp90 allows the assumption that in this case a similar interaction of Hsp90 proteins with their respective TPR binding partners occurs (FIG. 7B). In concordance with the thermo-dynamic results it follows that regarding the TPR binding, a high level of sequential conservation for the last five amino acids (consensus sequence: MEEVD) is important.

A structural comparison (FIG. 5) also explains in which manner the specific binding of Hsp70 and Hsp90 to the TPR domain of Hop protein is achieved. While the last two amino acid residues of Hsp70 and Hsp90 peptides are bound at comparable or equivalent positions in their respective TPR domains, in the case of the peptides placed in sections in a further N-terminal direction of the peptides, a binding to significantly different TPR areas can be seen. While the completely conserved VD part of the two peptides in each of the two TPR domains is positioned as a result of the electrostatic interactions via the “two carboxylate clamp”, the divergent N-terminal areas of the peptide utilise different hydrophobic caves or recesses in the TPR domains for the selectivity of the binding. Only a weak, non-specific binding to TPR1 was observed for the C-terminal domain of Hsp90 (C90) and for peptides derived from C90, whereby the affinities are of the same order of magnitude as they were observed for a peptide with a completely conserved EEVD sequence (FIG. 6A). The interaction of domain TPR2A with C70 or peptides derived from the C-terminal binding domain of Hsp70 (FIG. 6B) leads to similar results. In this case short C70 peptides bind to the domain TPR2A with an affinity which has also been observed for the tetra-peptide EEVD, while the C70 domain binds with a significantly reduced affinity (250•M). This can be attributed to the existence of steric problems between the C70 domain and TPR2A.

TABLE 1
Statistical details regarding the collection of data and the refinement for
the TPR1 complex

| | |
|---|---|
| Data collection | |
| X-ray source | NSLS, X12B^{b} |
| | · = 0.949 Å, T = 100 K |
| Space group | P4₁ |
| Cell parameters | a = 75.47 Å, c = 42.89 Å |
| Number of molecules/asymmetrical unit | 2 |
| Number of reflexes/individual reflexes | 146181/30804 |
| Resolution (Å) | 20.0-1.60 (1.66-1.60) |
| Completeness of data set (%) | 95.8 (75.2) |
| <I>/<·₁> | 32.5 (3.3) |
| R_{sym} (%) | 4.1 (30.5) |
| Model refinement^{d} | |
| Resolution (Å) | 20.0-1.60 |
| Number of reflexes used | 30804 |
| Number of reflexes in R_{free}set | 3100 |
| R_{work} (%)^{e} | 18.2^{f} |
| R_{free} (%)^{e} | 21.6^{f} |
| Number of protein/ligand atoms | 1840/120 |
| Number of hetero atoms | 36 |
| Number of solvent molecules | 245 |
| Standard mean deviation of bond length | 0.008 |
| (Å) | 1.3 |
| Standard mean deviation of bond angle (°) | |

^{a}Values as defined in SCALEPACK (Otwinoswki and Minor, 1997)
^{b}National Synchrotron Light Source in Brookhaven, beamline X12B
^{c}Drop in higher resolution shell due to rectangular detector shape
^{d}Values as defined in SHELXL (Sheldrick 1997) for peptide free/CNS (Brüniger et al. 1998) for complex
^{e}No sigma cutoff
^{f}No NCS restrains

TABLE 2
Statistical details regarding the collection of data and the refinement for the TPR2A
complex

Data collection

| Space group | C2 | | | | |
|---|---|---|---|---|---|
| Unit cell | a = 73.28 Å, b = 48.27, c = 38.06 Å, b = 91.30° | | | | |
| | Native^{a} | Ni1^{b} | Ni2^{b} | Se1^{b} | Se2^{b} |
| X-ray source | ESRF, ID14-3^{c} | | DESY, BW6^{d} | | |
| Resolution (Å) | 15-1.9 (1.95-1.90) | | 17-2.1 (2.14-2.10) | | |
| Wavelength | I = 0.9402 | I = 1.4828 | I = 1.4840 | I = 0.9793 | I = 0.9798 |
| (Å) | | | | | |
| I/s₁ | 22.8 (9.0) | 22.3 (10.2) | 22.8 (10.1) | 23.8 (11.1) | 25.6 (10.6) |
| Completeness | 97.1 (91.7) | 93.0 (94.9) | 98.8 (98.6) | 96.8 (96.8) | 88.0 (90.1) |
| of data set (%) | | | | | |
| R_{merge} (%) | 3.8 (6.8) | 2.8 (8.3) | 3.3 (10.5) | 3.3 (8.8) | 3.1 (9.0) |
| MAD Phasing^{f} | | | | | |

| Anomalous scatterer | 1 Ni, 2 Se | | | |
|---|---|---|---|---|
| Resolution (Å) | 17.0-2.1 | | | |
| Figure of merit | 0.74 | | | |
| R_{cullis}_ano | 0.64 | 0.87 | 0.65 | 0.85 |
| Phasing power centric | 0.48 | — | 1.51 | 1.30 |
| Phasing power acentric | 0.65 | — | 2.46 | 2.27 |
| Model Refinements^{g} | | | | |

| | | | |
|---|---|---|---|
| Resolution (Å) | 10-1.9 | Number of protein/ligand | 1042/44 |
| Number of reflexes for R_{work} | 9217 | atoms | 1 |
| Number of reflexes for R_{free} | 1003 | Number of hetero atoms (Ni) | 152 |
| R_{work} (%)^{h} | 18.1 | Number of solvent molecules | 0.008 |
| R_{free} (%)^{h} | 21.9 | Standard mean deviation of | 1.2 |
| | | bond length( Å) | |
| | | Standard mean deviation of | |
| | | bond angle (°) | |

^{a}Values as defined in XDS (Kabsch, 1993),
^{b}Values as defined in SCALEPACK (Otwinoski and Minor, 1997),
^{c}European Synchrotron Radiation Facility in Grenoble, beamline ID14-3
^{d}Deutsches Elektronen Synchrotron in Hamburg, beamline BW6,
^{e}Bijvoet pairs separated except for the native data set,
^{f}Unweighted values as defined in MLPHARE (CCP4, 1994),
^{g}Values as defined in CNS (Brunger et al., 1998),
^{h}No sigma cutoff.

Patent claims:

1. A 3-dimensional (3D) structure of a polypeptide, characterized as containing at least one amino acid sequence of a tetratrico peptide repeat (TPR) structure motif of a Hop protein, or a derivative thereof.

2. The 3D structure of claim 1, wherein the at least one amino acid sequence is selected from the group consisting of sequence (1), (2), (3), (4), (5), (6), (7), (8) and (9) (SEQ ID NO:19-27) shown in FIG. 3A.

3. The 3D structure of claim 1, wherein the at least one amino acid sequence is selected from the group consisting of domain TPR1, TPR2A or TPR2B of a Hop protein, or a fragment or derivative thereof.

4. The 3D structure of claim 3, wherein the at least one amino acid sequence is selected from the group consisting of sequence (1), (2) and (3) (SEQ ID NO:28-30), as shown in FIG. 3B.

5. The 3D structure of claim 4, wherein the at least one amino acid sequence is a TPR-structure motif or a TPR-domain corresponding to a Hop protein of eukaryotic origin.

6. The 3D structure of claim 1, wherein the 3D structure comprises the polypeptide and at least one other compound which binds to the polypeptide as a ligand.

7. The 3D structure of claim 6, wherein the ligand is not a molecule occurring physiologically.

8. The 3D structure of claim 1, wherein the 3D structure comprises a polypeptide and at least one physiological ligand or fragment thereof.

9. The 3D structure of claim 1, wherein the 3D structure features a polypeptide and at least one ligand or a section of a ligand, and wherein the ligand binds to the TPR structure motif.

10. The 3D structure of claim 9, wherein the ligand is a polypeptide, oligopeptide, depeptide or a synthetically modified derivative of a poly-, oligo- or dipeptide.

11. The 3D structure of claim 6, wherein the ligand contains an amino acid sequence or a fragment of a sequence from a chaperone protein or a derivative of a chaperone protein.

12. The 3D structure of claim 11, wherein the ligand comprises a fragment of a C-terminal amino acid sequence of a chaperone protein.

13. The 3D structure of claim 12, wherein the chaperone protein is Hsp70 and/or Hsp90, or a fragment thereof.

14. The 3D structure of claim 11, wherein the ligand is an inhibitor of the adapter function of a Hop protein.

15. The 3D structure of claim 10, wherein the ligand is an inhibitor of the interactions between Hop and Hsp70 and/or Hop and Hsp90.

16. The 3D structure of claim 1, wherein the 3D structure a crystal structure.

17. The 3D structure of claim 16, wherein the crystal structure contains heavy metal ions.

18. The 3D structure of claim 17, wherein the crystal structure contains at least one TPR structure motif of a Hop protein selected from the group of sequences consisting of (1), (2), (4), (5), (7) and (8) (SEQ ID NO:19, 20, 22, 23, 25, 26) shown in FIG. 3A with structure coordinates as shown in FIGS. 3C and 3D, or 3E.

19. The 3D structure of claim 17, wherein the crystal structure contains at least one TPR domain of a Hop protein selected from the group consisting of sequence (1) and (2) (SEQ ID NO:28-29) shown in FIG. 3B with the respective structure coordiates shown in FIGS. 3C and 3D for TPR1, and FIG. 3E for TPR2A.

20. A method to produce a crystal with unit cells, which contain in the asymmetric unit at least on 3-dimensional (3D) structure of a polypeptide and, optionally, at least one other compound, comprising
(a) applying the polypeptide to a coating in an extrusion system;
(b) cleaning and re-concentrating the polypeptide coating;
(c) dissolving the polypeptide concentrate in a suitable buffer system; and
(d) initiating crystallization.

21. A method for identifying a compound capable of acting as an inhibitor of the interaction between a Hop protein and a chaperone protein, wherein the chaperon protein is Hsp70 or Hsp90, comprising:
(a) obtaining the 3-dimensional (3D) structure of claim 1;
(b) representing the 3D structure three-dimensionally by way of structure coordinates; and
(c) selecting steric attributes and/or functional groups in such a manner that interactions between the compound and the 3D structure of the polypeptide are formed or, optionally, optimized.

22. A method for identifying a compound capable of acting as an inhibitor of an interaction between a Hop protein and at least one chaperone protein, wherein the chaperone protein is Hsp70 or Hsp90, comprising:
(a) constructing a biological test system for a potential inhibitor;
(b) determining the ability of a test compound to act as an inhibitor in the biological test system of (a),
(c) determining the conformation of an inhibitory test compound;
(d) depicting the 3-dimensional (3D) structure of claim 1 by its structure-coordinates; and
(e) inserting the inhibitory compound into the 3D structure of (d).

23. A DNA sequence encoding a partial sequence of a protein selected from the group consisting of TTC1, TTC2, TTC3, TTC4, IRSP, SGT and KIAA0719 as shown in FIG. 3, whereby the partial sequence binds to Hsp70 and/or Hsp90.

24. An amino acid sequenceencoded by the DNA sequence of claim 23.
